(19) United States
(12) Patent Application Publication
(10) Pub. No.: US 2026/0301880 A1
(43) Pub. Date: Oct. 1, 2026

(54) NOSE-TO-BRAIN PHYSIOLOGICALLY BASED PHARMACOKINETIC MODEL

(71) Applicant: APTARGROUP INC., Congers, NY (US)

(72) Inventor: William Ganley, South Wales (GB)

(21) Appl. No.: 19/474,253

(22) PCT Filed: Apr. 18, 2024
(86) PCT No.: PCT/US2024/025235

§ 371 Date: Oct. 10, 2025

Related U.S. Application Data
(60) Provisional application No. 63/461,111, filed on Apr. 21, 2023.

(51) Int. Cl. G16C 20/30; G16C 20/70; G16H 15/00; G16H 20/10

(52) U.S. Cl. CPC G16C 20/30; G16C 20/70; G16H 15/00; G16H 20/10

(57) ABSTRACT

The present disclosure relates to the building and use of physiologically-based pharmacokinetic models for predicting absorption/distribution of a nasally administered compound of interest.

### CROSS-REFERENCE TO RELATED APPLICATIONS

This application claims the benefit of priority to U.S. Application No. 63/461,111, filed on Apr. 21, 2023, the contents of which are hereby incorporated by reference.

### TECHNICAL FIELD

The present invention relates to computer-implemented nose-to-brain pharmacokinetic simulation models.

### BACKGROUND

Pharmacodynamics refers to the study of fundamental and/or molecular interactions between drug and body constituents, which through a subsequent series of events results in a pharmacological response. For most drugs, the magnitude of a pharmacological effect depends on time-dependent concentration of a drug at the site of action (e.g., target receptor-ligand/drug interaction). Factors that influence rates of delivery and disappearance of the drug to or from the site of action over time include absorption, distribution, metabolism, and elimination. The study of factors that influence how drug concentration varies with time is the subject of pharmacokinetics.

Each step of drug absorption, distribution, metabolism, and elimination can be described mathematically as a rate process. Many of these biochemical processes involve first order or pseudo-first order rate processes. In other words, the rate of reaction is proportional to drug concentration. For instance, pharmacokinetic data analysis is based on empirical observations after administering a known dose of drug and simulation of the event by either descriptive equations or mathematical (compartmental) models. This permits summarization of the experimental measures (plasma/blood level-time profile) and prediction under certain experimental conditions.

Simulations (i.e., computer-implemented models) have been used in pharmacokinetics to bring about solutions to complex pharmacokinetic equations and modeling of pharmacokinetic processes. While tools to develop and implement pharmacokinetic models exist, pharmacokinetic models and computer systems developed to date have not permitted sufficient predictability of the pharmacokinetic fate of nasally administered drugs to brain tissue in a mammal. Accordingly, there is a need to develop a comprehensive, physiologically-based pharmacokinetic model that is capable of predicting pharmacokinetics of a drug that is nasally inhaled.

### SUMMARY

The present disclosure relates to the generation and building of whole-body physiologically-based pharmacokinetic model (PBPK) models. The PBPK models are multi-compartment mathematical models that include operably linked components: (i) differential equations for one or more of fluid transit, fluid absorption, mass transit, mass dissolution, mass solubility, and mass absorption for one or more compartments of the mammalian system of interest; and (ii) initial parameter values for the differential equations corresponding to physiological parameters and compound-specific parameters, for one or more compartments of the mammalian system of interest. Initially, the PBPK models are built based upon known compounds that have known information pertaining to their pharmacokinetics. Also provided herein are methods for using the PBPK models for predicting pharmacokinetic data of new and/or candidate compounds in a mammalian, e.g., human, system of interest. The PBPK models and methods of use rely on computer-implemented systems and/or simulation engines that have the capability of solving differential equations.

Accordingly, in a first aspect, the disclosure provides methods, e.g., computer-implemented methods, of generating a trained nose-to-brain physiologically based pharmacokinetic (PBPK) model of delivery of a drug to brain tissue via nasal inhalation, the method including: (a) generating a multi-compartment PBPK model comprising a plurality of separate body compartments; (b) adding to the multi-compartment PBPK model a brain compartment and a nasal cavity compartment comprising one or more separate nasal sub-compartments, thereby generating an initial nose-to-brain PBPK model; (c) parameterizing the initial nose-to-brain PBPK model to include data for a plurality of physiological parameters for the plurality of separate body compartments and/or one or more or all of the separate nasal sub-compartments; (d) parameterizing the initial nose-to-brain PBPK model to include compound-specific parameters for a drug with known pharmacokinetic data for each body compartment and each nasal sub-compartment, to generate a parameterized nose-to-brain PBPK model; (e) running the parameterized nose-to-brain model and generating a report comprising drug concentration data for one or more of the body compartments and/or one or more of the separate nasal sub-compartments at one or more time points; and (f) analyzing and comparing the drug concentration data in the report with the known pharmacokinetic data for the drug, and updating the parameterized nose-to-brain model to obtain a trained nose-to-brain model.

In some embodiments, the plurality of compartments of the multi-compartment physiologically based pharmacokinetic model represent one or more or all of arterial blood, lung, adipose, bone, brain, heart, kidney, nose, muscle, skin, gut, spleen, liver, rest of body compartments, and/or venous blood.

In some embodiments, the one or more separate nasal sub-compartments represent one or more or all of: a nose area, a nasal floor, nasal turbinates, an olfactory region, or a rhinopharynx region.

In some embodiments, the brain compartment comprises separate sub-compartments representing one or more or all of brain blood outside of the blood brain barrier, brain mass representing brain tissue, cranial cerebrospinal fluid (CSF), and/or spinal CSF.

In some embodiments, at least one of the nasal sub-compartments are further represented as comprising a mucus layer where the drug is assumed to be deposited, an epithelial layer which permits permeation into deeper tissue, and a subepithelial layer perfused by blood vessels that allow transport of the drug into systemic circulation.

In certain embodiments, the physiological parameters for at least one compartment and sub-compartment are selected from the parameters recited in Table 1.

In some embodiments, the compound-specific parameters for at least one compartment and sub-compartment are selected from the parameters recited in Table 2.

In some embodiments, the compound-specific parameters for the drug comprises: P (permeability in the nasal epithelium), Log P (Log of the octanol/water partition coefficient), pKa (Negative log of the acid dissociation constant), fu (fraction of the drug dissolved in blood plasma that is unbound to plasma proteins), R (blood/plasma ratio), Clₕ (hepatic clearance), and mass deposited in at least one region of the nose.

In some embodiments, the body compartments of the multi-compartment PBPK model are represented by differential equations representing one or more or all of: (i) concentration of the drug in one or more of lung, adipose, bone, heart, kidneys, muscle, skin, or the rest of the body, and (ii) plasma concentration of the drug in one or more or all of spleen, liver, gut, arterial plasma and/or venous with respect to time as functions of the volume of the lung tissue, and wherein the differential equations are functions of one or more or all of the following parameters: blood flow to the lung; concentration of the drug in the venous plasma; blood:plasma ratio; tissue:plasma partition coefficient of the drug in each tissue type; fraction of the drug unbound in tissue; clearance rate of the drug from tissue; volume of each tissue or plasma type; concentrations of the drug in each tissue or plasma type; blood flow rates to each tissue type; hepatic clearance rate; and/or clearance rate of the drug from the blood. In some embodiments, the differential equations are one or more or all of the following:

$${V}_{lung} \frac{{dC}_{lung}}{di} = {Q}_{lung} ( {C}_{ven} - \frac{{RC}_{lung}}{{K}_{pu , lung}} ) - \frac{{f}_{u} {Cl}_{tissue} {RC}_{lung}}{{K}_{pu , lung}}$$

$${V}_{T} \frac{{dC}_{T}}{dt} = {Q}_{T} ( {C}_{art} - \frac{R {C}_{T}}{{K}_{pu , T}} ) - \frac{{f}_{u} {Cl}_{tissue} {RC}_{T}}{{K}_{pu , T}}$$

$${V}_{spleen} \frac{{dC}_{spleen}}{di} = {Q}_{spleen} ( {C}_{art} - \frac{R {C}_{spleen}}{{K}_{pu , spleen}} )$$

$${V}_{gut} \frac{{dC}_{gut}}{dt} = {Q}_{gut} ( {C}_{art} - \frac{{RC}_{gut}}{{K}_{pu , gut}} )$$

$${V}_{liver} \frac{{dC}_{liver}}{dt} = {Q}_{liver} {C}_{art} + {Q}_{spleen} \frac{R {C}_{spleen}}{{K}_{pu , spleen}} + {Q}_{gut} \frac{{RC}_{gut}}{{K}_{pu , gut}} - ( {Q}_{liver} + {Q}_{spleen} + {Q}_{gut} ) \frac{R {C}_{liver}}{{K}_{pu , liver}} - \frac{{f}_{u} {Cl}_{h} {RC}_{liver}}{{K}_{pu , liver}}$$

$${V}_{art} \frac{d {C}_{art}}{dt} = {Q}_{lung} ( \frac{{RC}_{lung}}{{K}_{pu , lung}} - {C}_{art} ) - {Cl}_{blood} {C}_{art} , and$$

$${V}_{ven} \frac{{dC}_{ven}}{di} = \sum {Q}_{T} ( \frac{R {C}_{T}}{{K}_{pu , T}} - {C}_{ven} ) + ( {Q}_{liver} + {Q}_{spleen} + {Q}_{gut} ) ( \frac{R {C}_{liver}}{{K}_{pu , liver}} - {C}_{ven} ) - {Cl}_{blood} {C}_{ven}$$

wherein V_{lung} is the volume of the lung tissue; C_{lung} is the concentration of the drug in the lung tissue; Q_{lung} is the blood flow to the lung; Cᵥₑₙ is the concentration of the drug in the venous plasma; R is the blood:plasma ratio; K_{pu,lung} is the tissue:plasma partition coefficient of the drug in the lung; fᵤ is the fraction of the drug unbound in tissue; Clₜᵢₛₛᵤₑ is the clearance rate of the drug from tissue; T is adipose, bone, heart, kidneys, muscle, skin, and the rest of the body; Vₛₚₗₑₑₙ is the volume of the spleen tissue; Cₛₚₗₑₑₙ is the concentration of the drug in the spleen tissue; Qₛₚₗₑₑₙ is the blood flow to the spleen; K_{pu,spleen} is the tissue:plasma partition coefficient of the drug in the spleen; V_{gut} is the volume of the gut tissue; C_{gut} is the concentration of the drug in the gut tissue; Q_{gut} is the blood flow to the gut; K_{pu,gut} is the tissue:plasma partition coefficient of the drug in the gut; Vₗᵢᵥₑᵣ is the volume of the liver tissue; Cₗᵢᵥₑᵣ is the concentration of the drug in the liver tissue; Oliver is the blood flow to the liver; K_{pu,liver} is the tissue:plasma partition coefficient of the drug in the liver; Cₐᵣₜ is the concentration of the drug in the arterial plasma; Clₙ is the hepatic clearance rate; and Cl_{blood} is the clearance from the blood.

In some embodiments, the nasal cavity compartments are represented by differential equations representing one or more or all of: (i) concentration of the drug in the nasal mucus, (ii) concentration of the drug in the nasal epithelium, and (iii) concentration of the drug in the nasal subepithelium, and wherein the differential equations are functions of one or more of the following parameters: permeability in the nasal epithelium; area of each region in the nasal cavity; tissue:plasma partition coefficient of the drug in the nose; mucociliary clearance rate in each region of the nasal cavity; rate of transport from the olfactory tissue to the cerebrospinal fluid via the intracellular and extracellular pathways through the olfactory and/or trigeminal nerves; rate of transport from the cerebrospinal fluid to the olfactory tissue via the intracellular and extracellular pathways through the olfactory and/or trigeminal nerves; and/or fraction of the drug unbound in the cranial CSF and/or the concentration in the cranial CSF. In some embodiments, the differential equations are one or more of the following:

$${V}_{muc , i} \frac{{dC}_{muc , i}}{dt} = - {PA}_{i} ( {C}_{muc , i} {f}_{u , muc} - {K}_{pu , nose} {C}_{ep , i} ) + {k}_{mcc , in , i} {C}_{muc , i - 1} - {k}_{mcc , out , i} {C}_{muc , i}$$

$${V}_{ep , i} \frac{{dC}_{ep , i}}{dt} = {PA}_{i} ( {C}_{muc , i} {f}_{u , muc} - {K}_{pu , nose} {C}_{ep , i} ) - {PA}_{i} ( {K}_{pu , nose} {C}_{ep , i} - {K}_{pu , nose} {C}_{sub , i} ) - \frac{{f}_{u} {Cl}_{tissue} {RC}_{ep , i}}{{K}_{pu , nose}} - {k}_{brain , in} {K}_{pu , nose} {C}_{ep , i} + {k}_{brain , out} {f}_{u , ccsf} {C}_{ccsf}$$

$${V}_{sub , i} \frac{{dC}_{sub , i}}{dt} = {PA}_{i} ( {K}_{pu , nose} {C}_{ep , i} - {K}_{pu , nose} {C}_{sub , i} ) - {Q}_{nose , i} ( {C}_{art} - \frac{R {C}_{sub , i}}{{K}_{pu , nose}} ) - \frac{{f}_{u} {Cl}_{tissue} {RC}_{sub , i}}{{K}_{pu , nose}} - {k}_{brain , in} {K}_{pu , nose} {C}_{ep , i} + {k}_{brain , out} {f}_{u , ccsf} {C}_{ccsf}$$

wherein V_{muc,i}, C_{muc,i}, V_{ep,i}, C_{ep,i}, V_{sub,i} and C_{sub,i} are the volume and drug concentration pairs in the nasal mucus, epithelium and sub-epithelium of the ith nasal region (referring to the nasal valve, floor, turbinates, turbinates, olfactory region and rhinopharynx); P is the permeability in the nasal epithelium; Aᵢ is the area of the ith nasal region; K_{pu,nose} is the tissue:plasma partition coefficient of the drug in the nose; k_{mcc,in,i} is the mucociliary clearance rate transporting mass into the ith region (equal to 0 for all regions apart from the rhinopharynx where it equals k_{mcc,ant}); k_{mcc,out,i} is the mucociliary clearance rate transporting mass out of the ith region (equal to k_{mcc,ant} for the nasal floor, turbinates and olfactory region and equal to k_{mcc,post} for the rhinopharynx); k_{brain,in} is the rate of transport from the olfactory tissue to the cerebrospinal fluid via the intracellular and extracellular pathways; k_{brain,out} is the rate of transport from the cerebrospinal fluid to the olfactory tissue via the intracellular and extracellular pathways (these two parameters are zero for the non-olfactory regions); f_{u,ccsf} is the fraction of the drug unbound in the cranial CSF; and C_{ccsf} is the concentration in the cranial CSF.

In certain embodiments, the brain compartments are represented by differential equations representing one or more or all of: (i) concentration of the drug in the brain blood, (ii) concentration of the drug in the brain mass, (iii) concentration of the drug in the cranial cerebrospinal fluid, and (iv) concentration of the drug in the spinal cerebrospinal fluid; and wherein the differential equations are functions of one or more of the following parameters: concentration of the drug molecule in the brain blood; blood flow to the brain; concentration of the compound in the arterial plasma; fraction of the compound unbound in tissue; clearance rate of the compound from tissue; product of the permeability and surface area between brain blood and brain mass; fraction of the drug unbound in the brain mass; concentration of blood in the brain mass; fraction of the drug unbound in the brain blood; product of the permeability and surface area between brain blood and cranial CSF; fraction of the drug unbound in the cranial CSF; concentration of the drug in the cranial CSF the flow rates from the cranial and spinal CSF to the brain blood; concentration of the drug in the spinal CSF; volume of the brain mass; product of the permeability and surface area between brain mass and cranial CSF; bulk flow from the brain mass to the cranial CSF; CSF shuttle flow between the cranial and spinal CSF; rate of transport from the olfactory tissue to the cerebrospinal fluid via the intracellular and extracellular pathways along the olfactory and/or trigeminal nerves; tissue:plasma partition coefficient of the drug in the nose; the drug concentration in the epithelium of each region of the nasal cavity; rate of transport from the cerebrospinal fluid to the olfactory tissue via the intracellular and extracellular pathways along the olfactory and/or trigeminal nerves; and/or the drug concentration in the sub-epithelium of each region of the nasal cavity and/or the volume of the spinal CSF. In some embodiments, the differential equations are:

$${V}_{brain} \frac{{dC}_{brain}}{dt} = {Q}_{brain} ( {C}_{art} - {RC}_{brain} ) - {f}_{u} {Cl}_{tissue} {RC}_{brain} + {P}_{b} {A}_{b} ( {f}_{u , bm} {C}_{bm} - {f}_{u , brain} {C}_{brain} ) + {P}_{c} {A}_{c} ( {f}_{u , ccsf} {C}_{ccsf} - {f}_{u , brain} {C}_{brain} ) + {Q}_{ssink} {C}_{scsf} + {Q}_{csink} {C}_{ccsf}$$

$${V}_{bm} \frac{{dC}_{bm}}{dt} = {P}_{b} {A}_{b} ( {f}_{u , brain} {C}_{brain} - {f}_{u , bm} {C}_{bm} ) + {P}_{e} {A}_{e} ( {f}_{u , ccsf} {C}_{ccsf} - {f}_{u , bm} {C}_{bm} ) - {Q}_{bulk} {C}_{bm}$$

$${V}_{ccsf} \frac{{dC}_{ccsf}}{dt} = {P}_{e} {A}_{e} ( {f}_{u , bm} {C}_{bm} - {f}_{u , ccsf} {C}_{ccsf} ) + {P}_{c} {A}_{c} ( {f}_{u , brain} {C}_{brain} - {f}_{u , ccsf} {C}_{ccsf} ) + {Q}_{bulk} {C}_{bm} + {Q}_{sout} {C}_{scsf} - {Q}_{sin} {C}_{ccsf} - {Q}_{csink} {C}_{ccsf} + {k}_{brain , in} {K}_{pu , nose} {C}_{ep , i} - {k}_{brain , out} {f}_{u , ccsf} {C}_{ccsf} + {k}_{brain , in} {K}_{pu , nose} {C}_{sub , i} - {k}_{brain , out} {f}_{u , ccsf} {C}_{ccsf}$$

$${V}_{scsf} \frac{{dC}_{scsf}}{dt} = {Q}_{sin} {C}_{ccsf} - {Q}_{sout} {C}_{scsf} - {Q}_{ssink} {C}_{scsf}$$

wherein V_{brain} is the volume of the brain blood; C_{brain} is the concentration of the drug molecule in the brain blood; Q_{brain} is the blood flow to the brain; Cₐᵣₜ is the concentration of the compound in the arterial plasma; fᵤ is the fraction of the compound unbound in tissue; Clₜᵢₛₛᵤₑ is the clearance rate of the compound from tissue; P_{b}A_{b} is the product of the permeability and surface area between brain blood and brain mass; f_{u,bm} is the fraction of the drug unbound in the brain mass; C_{bm} is the concentration of blood in the brain mass; f_{u,brain} is the fraction of the drug unbound in the brain blood; P_{c}A_{c} is the product of the permeability and surface area between brain blood and cranial; f_{u,cesf} is the fraction of the drug unbound in the cranial CSF; C_{ccsf} is the concentration of the drug in the cranial CSF; Q_{csink} and Qₛₛᵢₙₖ are the flows from the cranial and spinal CSF to the brain blood respectively; C_{scsf} is the concentration of the drug in the spinal CSF; V_{bm} is the volume of the brain mass; PₑAₑ is the product of the permeability and surface area between brain mass and cranial CSF; Q_{bulk} is the bulk flow from the brain mass to the cranial CSF; Qₛₒᵤₜ and Qₛᵢₙ are the CSF shuttle flow between the cranial and spinal CSF; K_{brain,in} is the rate of transport from the olfactory tissue to the cerebrospinal fluid via the intracellular and extracellular pathways; K_{pu,nose} is the tissue:plasma partition coefficient of the drug in the nose; C_{ep,i} is the drug concentration in the epithelium of the ith nasal region (referring to the nasal valve, floor, turbinates, turbinates, olfactory region and rhinopharynx); k_{brain,out} is the rate of transport from the cerebrospinal fluid to the olfactory tissue via the intracellular and extracellular pathways; C_{sub,i} is the drug concentration in the sub-epithelium of the ith nasal region (referring to the nasal valve, floor, turbinates, turbinates, olfactory region and rhinopharynx); and V_{scsf} is the volume of the spinal CSF.

In some embodiments, the compound-specific parameters of step (c) are based on pre-clinical studies for the known drug. In some instances, the pre-clinical studies comprise in vitro cell assays, in vitro protein assays (e.g., binding data), and/or in vivo studies.

In some embodiments, the physiological-specific parameters are obtained from literature and/or database searches.

In certain embodiments, wherein step (e) comprises solving the differential equations representing the compartments of the PBPK model. In some instances, the solving is done with a differential equation solver.

In some embodiments, the pharmacokinetic data of step (f) is based on clinical data for the known drug. In some instances, further comprising adjusting one or more of the physiological parameters or the compound-specific parameters thereby training the model.

In some embodiments, wherein the PBPK model is species specific. In some instances, the species is human. In some instances, the species is a non-human primate.

In certain embodiments, the known drug is sumatriptan.

In another aspect, the disclosure provides methods, e.g., computer-implemented methods, of predicting results for transport of a new drug to brain tissue via nasal inhalation. The methods include: (a) accessing a trained nose-to-brain PBPK model generated using the method of any one of the above described methods and parameterizing the nose-to-brain PBPK model with compound-specific parameters known about the drug; (b) receiving and inputting nasal cast deposition data for the drug into the trained nose-to brain PBPK model of step (a); (c) using a differential equation solver to solve the equations of the trained nose-to-brain PBPK model; (d) obtaining a report comprising drug concentration data from the trained nose-to-brain model for one or more or all of the compartments and one or more or all of the sub-compartments at one or more time points; and (e) providing a prediction of transport results for the drug to brain tissue via nasal inhalation based on pharmacokinetic data for the brain compartments based on the report.

In some embodiments, the compound-specific parameters of step (a) are based on pre-clinical studies. In some embodiments, the pre-clinical studies comprise in vitro cell assays, in vitro protein assays (e.g., binding data), and/or in vivo studies.

In some embodiments, nasal cast deposition data comprises a mass of the drug deposited in each region of the nasal cast.

In some embodiments, step (a) further comprises updating the nose-to-brain PBPK model using clinical data known about the drug.

In some embodiments, the clinical data is intravenous data, nasal data, or both.

In another aspect, this disclosure also provides systems including: a memory to store instructions that are executable; and one or more processing devices coupled to the memory, wherein the one or more processing devices are configured to execute the instructions to perform operations comprising: (a) generating a multi-compartment PBPK model comprising a plurality of separate body compartments; (b) adding to the multi-compartment PBPK model a brain compartment and a nasal cavity compartment comprising one or more separate nasal sub-compartments, thereby generating an initial nose-to-brain PBPK model; (c) parameterizing the initial nose-to-brain PBPK model to include data for a plurality of physiological parameters for the plurality of separate body compartments and/or one or more or all of the separate nasal sub-compartments; (d) parameterizing the initial nose-to-brain PBPK model to include compound-specific parameters for a drug with known pharmacokinetic data for each body compartment and each nasal sub-compartment, to generate a parameterized nose-to-brain PBPK model; (e) running the parameterized nose-to-brain model and generating a report comprising drug concentration data for one or more of the body compartments and/or one or more of the separate nasal sub-compartments at one or more time points; and (f) analyzing and comparing the drug concentration data in the report with the known pharmacokinetic data for the drug, and updating the parameterized nose-to-brain model to obtain a trained nose-to-brain model.

In some embodiments, the plurality of compartments of the muti-compartment physiologically based pharmacokinetic model represent one or more or all of arterial blood, lung, adipose, bone, brain, heart, kidney, nose, muscle, skin, gut, spleen, liver, rest of body compartments, and/or venous blood.

In some embodiments, the brain compartment includes separate sub-compartments representing one or more or all of brain blood outside of the blood brain barrier, brain mass representing brain tissue, cranial cerebrospinal fluid (CSF), and/or spinal CSF.

In some embodiments, the nasal sub-compartments represent one or more or all of a nose area, a nasal floor, nasal turbinates, an olfactory region, and/or a rhinopharynx region and at least one is further represented as comprising a mucus layer where the drug is assumed to be deposited, an epithelial layer which permits permeation into deeper tissue, and a subepithelial layer perfused by blood vessels that allow transport of the drug into systemic circulation.

In some embodiments, the physiological parameters for at least one compartment and sub-compartment are selected from the parameters recited in Table 1. In certain embodiments, the compound-specific parameters for at least one compartment and sub-compartment are selected from the parameters recited in Table 2.

In some embodiments, the compound-specific parameters for the drug comprises: P (permeability in the nasal epithelium), Log P (Log of the octanol/water partition coefficient), pKa (Negative log of the acid dissociation constant), fu (fraction of the drug dissolved in blood plasma that is unbound to plasma proteins), R (blood/plasma ratio), Clₕ (hepatic clearance), and mass deposited in at least one region of the nose.

In another aspect, this disclosure provides one or more non-transitory machine-readable storage media storing instructions that when executed perform operations including: (a) generating a multi-compartment PBPK model comprising a plurality of separate body compartments; (b) adding to the multi-compartment PBPK model a brain compartment and a nasal cavity compartment comprising one or more separate nasal sub-compartments, thereby generating an initial nose-to-brain PBPK model; (c) parameterizing the initial nose-to-brain PBPK model to include data for a plurality of physiological parameters for the plurality of separate body compartments and/or one or more or all of the separate nasal sub-compartments; (d) parameterizing the initial nose-to-brain PBPK model to include compound-specific parameters for a drug with known pharmacokinetic data for each body compartment and each nasal sub-compartment, to generate a parameterized nose-to-brain PBPK model; (e) running the parameterized nose-to-brain model and generating a report comprising drug concentration data for one or more of the body compartments and/or one or more of the separate nasal sub-compartments at one or more time points; and (f) analyzing and comparing the drug concentration data in the report with the known pharmacokinetic data for the drug, and updating the parameterized nose-to-brain model to obtain a trained nose-to-brain model.

In some embodiments, the plurality of compartments of the muti-compartment physiologically based pharmacokinetic model represent one or more or all of arterial blood, lung, adipose, bone, brain, heart, kidney, nose, muscle, skin, gut, spleen, liver, rest of body compartments, and/or venous blood.

In certain embodiments, the brain compartment includes separate sub-compartments representing one or more or all of brain blood outside of the blood brain barrier, brain mass representing brain tissue, cranial cerebrospinal fluid (CSF), and/or spinal CSF.

In some embodiments, the nasal sub-compartments represent one or more or all of a nose area, a nasal floor, nasal turbinates, an olfactory region, and/or a rhinopharynx region and at least one is further represented as comprising a mucus layer where the drug is assumed to be deposited, an epithelial layer which permits permeation into deeper tissue, and a subepithelial layer perfused by blood vessels that allow transport of the drug into systemic circulation.

In some embodiments, the physiological parameters for at least one compartment and sub-compartment are selected from the parameters recited in Table 1. In certain embodiments, the compound-specific parameters for at least one compartment and sub-compartment are selected from the parameters recited in Table 2.

In some embodiments, the compound-specific parameters for the drug comprises: P (permeability in the nasal epithelium), Log P (Log of the octanol/water partition coefficient), pKa (Negative log of the acid dissociation constant), fu (fraction of the drug dissolved in blood plasma that is unbound to plasma proteins), R (blood/plasma ratio), Clₕ (hepatic clearance), and mass deposited in at least one region of the nose.

In another aspect, this disclosure also provides trained nose-to-brain physiologically based pharmacokinetic (PBPK) models of delivery of a drug to brain tissue via nasal inhalation, wherein the trained nose-to-brain PBPK model is (1) encoded on one or more non-transitory machine-readable storage media and is (2) generated by operations including: (a) generating a multi-compartment PBPK model comprising a plurality of separate body compartments; (b) adding to the multi-compartment PBPK model a brain compartment and a nasal cavity compartment comprising one or more separate nasal sub-compartments, thereby generating an initial nose-to-brain PBPK model; (c) parameterizing the initial nose-to-brain PBPK model to include data for a plurality of physiological parameters for the plurality of separate body compartments and/or one or more or all of the separate nasal sub-compartments; (d) parameterizing the initial nose-to-brain PBPK model to include compound-specific parameters for a drug with known pharmacokinetic data for each body compartment and each nasal sub-compartment, to generate a parameterized nose-to-brain PBPK model; (e) running the parameterized nose-to-brain model and generating a report comprising drug concentration data for one or more of the body compartments and/or one or more of the separate nasal sub-compartments at one or more time points; and (f) analyzing and comparing the drug concentration data in the report with the known pharmacokinetic data for the drug, and updating the parameterized nose-to-brain model to obtain a trained nose-to-brain model.

In some embodiments, the trained nose-to-brain PBPK model is configured to: (a) receive one or more compound-specific parameters known about a drug; (b) be solved by a differential equation; and (c) produce a report comprising drug concentration data from the trained nose-to-brain model for one or more or all of the compartments and one or more or all of the sub-compartments at one or more time points; and (e) providing a prediction of transport results for the drug to brain tissue via nasal inhalation based on pharmacokinetic data for the brain compartments based on the report.

In some embodiments, the plurality of compartments of the muti-compartment physiologically based pharmacokinetic model represent one or more of arterial blood, lung, adipose, bone, brain, heart, kidney, nose, muscle, skin, gut, spleen, liver, rest of body compartments, and/or venous blood.

In certain embodiments, the brain compartment includes separate sub-compartments representing one or more or all of brain blood outside of the blood brain barrier, brain mass representing brain tissue, cranial cerebrospinal fluid (CSF), and/or spinal CSF.

In some embodiments, the nasal sub-compartments represent one or more or all of a nose area, a nasal floor, nasal turbinates, an olfactory region, and/or a rhinopharynx region and at least one is further represented as comprising a mucus layer where the drug is assumed to be deposited, an epithelial layer which permits permeation into deeper tissue, and a subepithelial layer perfused by blood vessels that allow transport of the drug into systemic circulation.

In some embodiments, the physiological parameters for at least one compartment and sub-compartment are selected from the parameters recited in Table 1. In certain embodiments, the compound-specific parameters for at least one compartment and sub-compartment are selected from the parameters recited in Table 2.

In some embodiments, the compound-specific parameters for the drug comprises: P (permeability in the nasal epithelium), Log P (Log of the octanol/water partition coefficient), pKa (Negative log of the acid dissociation constant), fu (fraction of the drug dissolved in blood plasma that is unbound to plasma proteins), R (blood/plasma ratio), Clₕ (hepatic clearance), and mass deposited in at least one region of the nose.

In another aspect, the present disclosure provides systems including: a memory to store instructions that are executable; and one or more processing devices coupled to the memory, the one or more processing devices configured to execute the instructions to perform operations comprising: (a) accessing a trained nose-to-brain PBPK model generated using any one of the methods described above and parameterizing the nose-to-brain PBPK model with compound-specific parameters known about a drug; (b) receiving and inputting nasal cast deposition data for the drug into the trained nose-to brain PBPK model of step (a); (c) using a differential equation solver to solve the equations of the trained nose-to-brain PBPK model; (d) obtaining a report comprising drug concentration data from the trained nose-to-brain model for one or more or all of the compartments and one or more or all of the sub-compartments at one or more time points; and (e) providing a prediction of transport results for the drug to brain tissue via nasal inhalation based on pharmacokinetic data for the brain compartments based on the report.

In certain embodiments, the plurality of compartments of the muti-compartment physiologically based pharmacokinetic model represent one or more or all of arterial blood, lung, adipose, bone, brain, heart, kidney, nose, muscle, skin, gut, spleen, liver, rest of body compartments, and/or venous blood.

In some embodiments, the brain compartment includes separate sub-compartments representing one or more or all of brain blood outside of the blood brain barrier, brain mass representing brain tissue, cranial cerebrospinal fluid (CSF), and/or spinal CSF.

In some embodiments, the nasal sub-compartments represent one or more or all of a nose area, a nasal floor, nasal turbinates, an olfactory region, and/or a rhinopharynx region and at least one is further represented as comprising a mucus layer where the drug is assumed to be deposited, an epithelial layer which permits permeation into deeper tissue, and a subepithelial layer perfused by blood vessels that allow transport of the drug into systemic circulation.

In some embodiments, the physiological parameters for at least one compartment and sub-compartment are selected from the parameters recited in Table 1. In certain embodiments, the compound-specific parameters for at least one compartment and sub-compartment are selected from the parameters recited in Table 2.

In some embodiments, the compound-specific parameters for the drug comprises: P (permeability in the nasal epithelium), Log P (Log of the octanol/water partition coefficient), pKa (Negative log of the acid dissociation constant), fu (fraction of the drug dissolved in blood plasma that is unbound to plasma proteins), R (blood/plasma ratio), Clₕ (hepatic clearance), and mass deposited in at least one region of the nose.

Also provided herein are one or more non-transitory machine-readable storage media storing instructions that are executed to perform operations including: (a) accessing a trained nose-to-brain PBPK model generated using any one of the methods described above and parameterizing the nose-to-brain PBPK model with compound-specific parameters known about the drug; (b) receiving and inputting nasal cast deposition data for the drug into the trained nose-to brain PBPK model of step (a); (c) using a differential equation solver to solve the equations of the trained nose-to-brain PBPK model; (d) obtaining a report comprising drug concentration data from the trained nose-to-brain model for one or more or all of the compartments and one or more or all of the sub-compartments at one or more time points; and (e) providing a prediction of transport results for the drug to brain tissue via nasal inhalation based on pharmacokinetic data for the brain compartments based on the report.

In certain embodiments, the plurality of compartments of the muti-compartment physiologically based pharmacokinetic model represent one or more or all of arterial blood, lung, adipose, bone, brain, heart, kidney, nose, muscle, skin, gut, spleen, liver, rest of body compartments, and/or venous blood.

In some embodiments, the brain compartment includes separate sub-compartments representing one or more or all of brain blood outside of the blood brain barrier, brain mass representing brain tissue, cranial cerebrospinal fluid (CSF), and/or spinal CSF.

In some embodiments, the nasal sub-compartments represent one or more or all of a nose area, a nasal floor, nasal turbinates, an olfactory region, and/or a rhinopharynx region and at least one is further represented as comprising a mucus layer where the drug is assumed to be deposited, an epithelial layer which permits permeation into deeper tissue, and a subepithelial layer perfused by blood vessels that allow transport of the drug into systemic circulation.

In certain embodiments, the physiological parameters for at least one compartment and sub-compartment are selected from the parameters recited in Table 1. In some embodiments, the compound-specific parameters for at least one compartment and sub-compartment are selected from the parameters recited in Table 2.

In some embodiments, the compound-specific parameters for the drug comprises: P (permeability in the nasal epithelium), Log P (Log of the octanol/water partition coefficient), pKa (Negative log of the acid dissociation constant), fu (fraction of the drug dissolved in blood plasma that is unbound to plasma proteins), R (blood/plasma ratio), Clₕ (hepatic clearance), and mass deposited in at least one region of the nose.

### Definitions

Absorption: Transfer of a compound across a physiological barrier as a function of time and initial concentration. Amount or concentration of the compound on the external and/or internal side of the barrier is a function of transfer rate and extent.

Bioavailability: Fraction of an administered dose of a compound that reaches the sampling site and/or site of action. May range from zero to unity. Can be assessed as a function of time.

Compartment: Represents a tissue or organ or a sub-part of a given tissue or organ. Multiple compartments can be assembled to generate a model, e.g., a multi-compartment model, such as a whole-body model.

Compound: A compound is a chemical entity. As used herein the term “compound” is used interchangeably with drug.

Computer Readable Medium: Medium for storing, retrieving, and/or manipulating information using a computer. Includes optical, digital, magnetic mediums and the like; examples include portable computer diskette, CD-ROMs, hard drive on computer etc. Includes remote access mediums; examples include internet or intranet systems. Permits temporary or permanent data storage, access, and manipulation.

Data: Experimentally collected and/or predicted variables. May include dependent and independent variables.

Dissolution: Process by which a compound becomes dissolved in a solvent.

Permeability: Ability of a physiological barrier to permit passage of a substance. Refers to the concentration-dependent or concentration-independent rate of transport (flux), and collectively reflects the effects of characteristics such as molecular size, charge, partition coefficient and stability of a compound on transport. Permeability is substance and barrier specific.

Simulation Engine: Computer-implemented instrument that simulates behavior of a system using an approximate mathematical model of the system. Combines mathematical model with user input variables to simulate or predict how the system behaves. May include system control components such as control statements (e.g., logic components and discrete objects).

Solubility: Property of being soluble; relative capability of being dissolved.

Transport Mechanism: The mechanism by which a compound passes a physiological barrier of tissue or cells. Includes four basic categories of transport: passive paracellular, passive transcellular, carrier-mediated influx, and carrier-mediated efflux.

Parameterize/Parameterizing: Updating a model to include data for specific parameters. For example, data for specific physiological and/or specific compound (e.g., drug) parameters for one or more compartments (and/or sub-compartments) included in the model.

Train/Training: Updating and calibrating a model with real world data. For example, updating the model with known pharmacokinetic data for a specific compound (e.g., drug) for one or more compartments (and/or sub-compartments) included in the model.

The PBPK models described herein simulate the mass transfer from administration/deposition of a drug in the nose to the brain via two routes: direct transfer along the olfactory and tri-geminal nerves and absorption into the blood and subsequent transfer across the blood brain barrier. The models are parameterized using non-clinical data and therefore can be used to make predictions of concentrations in nasal and brain tissue of new drugs or drug candidates without the need for costly clinical studies. The models also provide organ (including brain)-specific targeting information that can inform the development of nasal spray products, can be used to predict safe and effective doses ahead of clinical studies to reduce risk, and can be continually improved through a product development to increase their credibility commensurate with the risk of decisions informed by their predictions.

The present PBPK models improve upon the current state of the art by explicitly simulating two simultaneous pathways of absorption into the brain: crossing of the blood brain barrier and transport through the olfactory and trigeminal nerves. This improves the accuracy of predicted brain exposure following nasal administration over other models that assume no transport through the olfactory and trigeminal nerves.

The present PBPK models can be used to support the development of nasal drug delivery products by predicting the influence of changes to a drug delivery device or formulation on the concentrations of drug in the blood plasma or brain at the early stages of development. In the absence of the nose-to-brain PBPK models disclosed herein, such information would not be known until clinical studies take place, by which time it is often too late to make substantial changes to the drug product. Gaining predicted clinical information early in development therefore reduces the risk of making formulation and device changes in early development and provides assurance of clinical performance before clinical studies take place, avoiding unnecessary clinical trials on humans and animals.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram of the systemic part of a muti-compartment physiologically based pharmacokinetic (PBPK) model including compartments representing the nose and brain, as described herein.

FIG. 2 is a schematic diagram showing the structure of sub-compartments of the nose compartment as added to the PBPK model.

FIG. 3 is a schematic diagram showing the structure of sub-compartments of the brain compartment including cerebrospinal fluid (CSF), brain tissue mass, and the blood-brain barrier, as added to the PBPK model.

FIG. 4 is a schematic diagram showing the structure of the nasal area, olfactory region, and the rhinopharynx sub-compartments of the nose compartment in the PBPK model.

FIG. 5A is a flow chart of an example of a system for generating PBPK models.

FIG. 5B is a flow chart and diagram of an example of a computer system for predicting delivery and/or pharmacokinetic data based on the PBPK model.

FIG. 6 is a schematic diagram of an example of system components that can be used to implement the models, systems, and methods described herein.

FIG. 7 is a graph showing the clinical (points) and predicted (lines, each representing a row in Table 6) pharmacokinetics using initial parameters (Table 4) in the PBPK model.

FIG. 8 is a graph showing the clinical (points) and predicted (lines, each representing a row in Table 6) pharmacokinetics using model parameters (see Table 5) in the PBPK model.

FIG. 9 is a graph showing the predicted nasal and intravenous plasma concentration vs. time profiles used to calculate the predicted nasal bioavailability of sumatriptan delivered using a unidose nasal spray device for liquid drugs, UDS-L from Aptar.

FIGS. 10A-10B are a series of graphs showing a PCA Biplot (FIG. 10A) and factor weighting vectors (FIG. 10B) from a nasal cast deposition and predicted pharmacokinetic data.

FIGS. 11A-11B are a series of graphs showing relationships between Cₘₐₓ (FIG. 11A) and AUC₀₋ₜ (FIG. 11B) and fractional deposition in the turbinates sub-compartment of the nose compartment.

FIGS. 12A-12B are a series of graphs showing the relationships between Cₘₐₓ (FIG. 12A) and AUC₀₋ₜ (FIG. 12B) and fractional deposition in the turbinates and nasal floor sub-compartments of the nose compartment.

FIGS. 13A-13B are a series of graphs showing the relationships between Cₘₐₓ (FIG. 13A) and AUC₀₋ₜ (FIG. 13B) and fractional deposition in the nasal floor sub-compartment.

FIGS. 14A-14B are a series of graphs showing the relationships between Cₘₐₓ (FIG. 14A) and AUC₀₋ₜ (FIG. 14B) and fractional deposition in the nasal floor sub-compartment.

FIG. 15 is a graph showing the relationships between turbinate deposition fraction and predicted Cₘₐₓ for different 100 μL devices and orientations. The lines are guides to the eye and are the closest approximation of the data points.

FIG. 16 is a graph showing the relationships between turbinate deposition fraction and predicted Cₘₐₓ for different 50 μL devices and orientations. The lines are guides to the eye and are the closest approximation of the data points.

FIG. 17 is a graph showing model simulations. Simulation 1 shows a model with no input mass, which produces a zero level plasma concentration, as expected. Simulation 2 shows a model with a 1 ng/mL initial plasma concentration of a hypothetical drug decaying to zero. Simulation 3 shows a model with an initial concentration in the olfactory mucus of 1 ng/mL decaying to zero.

FIGS. 18A-18B are a series of graphs showing predicted systemic plasma concentrations resulting from a single 0.5 mg dose of dihydroergotamine (DHE) using a high olfactory deposition (FIG. 18A) and a low olfactory deposition (FIG. 18B) pattern.

FIGS. 19A-19B are a series of graphs showing predicted brain compartment concentrations resulting from a single 0.5 mg dose of DHE using a high olfactory deposition (FIG. 19A) and a low olfactory deposition (FIG. 19B) pattern.

### DETAILED DESCRIPTION

Nasal drug delivery has existed for many decades and is a common route of administration for local acting therapies for diseases such as allergic rhinitis, sinusitis, and congestion, and it is increasingly being used and explored for systemic delivery, e.g., migraine, pain management, etc.

The nose-to-brain physiologically-based pharmacokinetic (PBPK) models disclosed herein are useful to help in assessing the distribution of a particular drug or drug candidate in the brain of a subject, e.g., a mammalian subject, such as a human. The new PBPK models offer advantages over the current state of the art PBPK models in that they explicitly simulate both transfer across the blood brain barrier and along the olfactory and trigeminal nerves. The current state of the art does not explicitly simulate transport along the olfactory and trigeminal nerves therefore underpredicting brain exposure. The new nose-to-brain PBPK models described herein therefore offer a more accurate predictive model of brain exposure following nasal administration.

### A. Building a Nose-to-Brain PBPK

a. Compartments

The muti-compartment PBPK models are mathematical models of multi-compartment physiological models of mammalian systems (e.g., the tissues/organs of a mammalian system). Compartments representing a particular anatomical segment or region (e.g., tissue, organ, or vasculature) of the body can be added or removed depending on the model's intended end use, such as when an isolated segment is examined, or when it is desired to account for parameters affecting bioavailability at additional sampling sites.

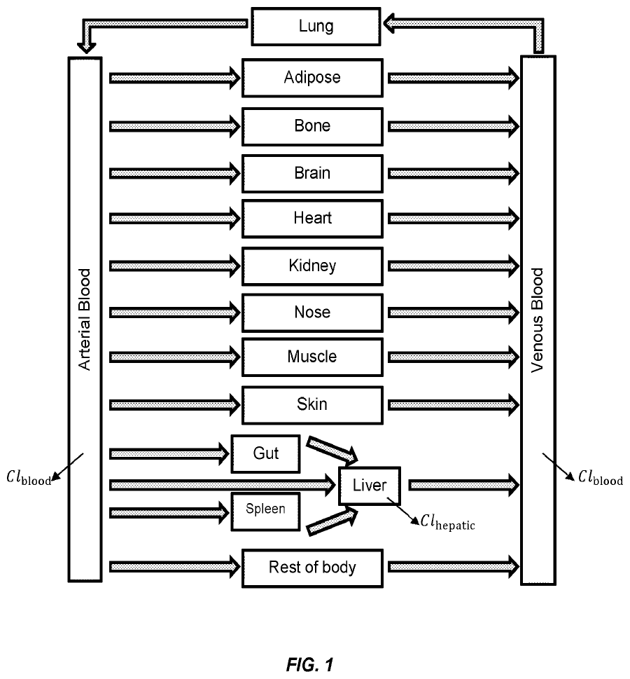

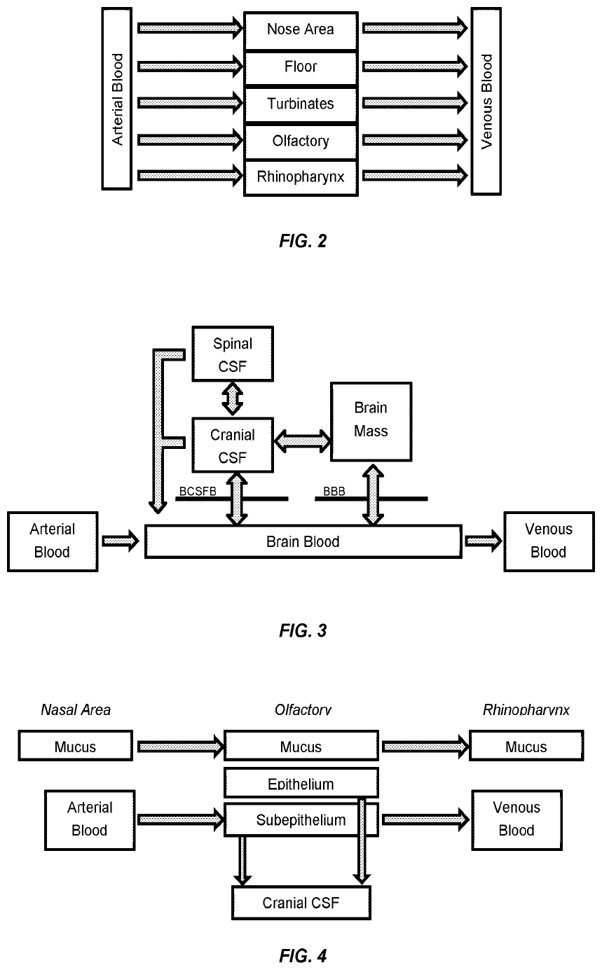

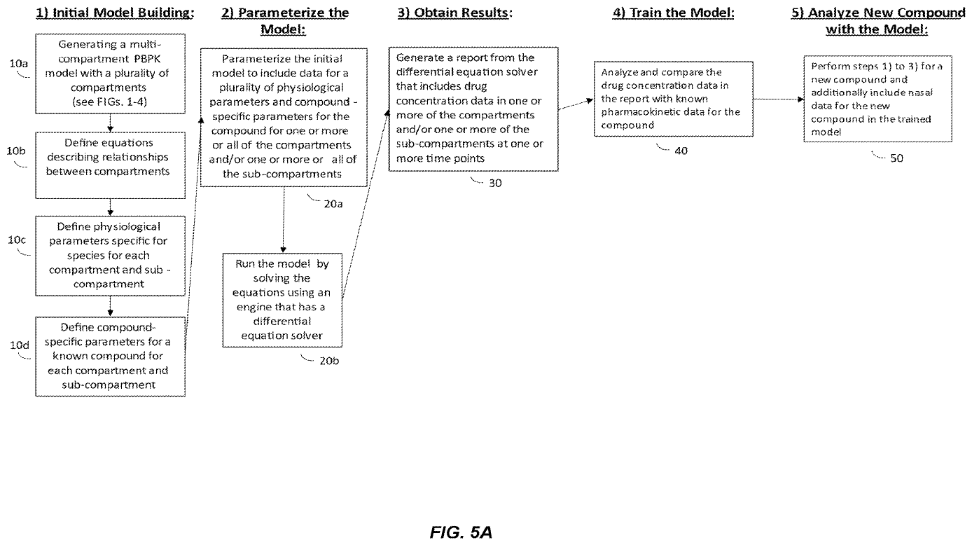

The muti-compartment PBPK models are schematically represented in FIG. 1, which shows one example of a muti-compartment model that was divided into the following compartments: arterial blood, lung, adipose, bone, brain, heart, kidney, nose, muscle, skin, gut, spleen, liver, rest of body compartments, and venous blood.

The nose compartment was further compartmentalized as shown in FIG. 2. Specifically, the nose compartment was divided into nose area, floor, turbinates, olfactory, and rhinopharynx sub-compartments. The nose compartment, subdivided in this way, has not previously been seen in such PBPK models.

The compartments and sub-compartments can be modified by factors that influence absorption such as mass, volume, surface area, concentration, permeability, solubility, fluid secretion/absorption, fluid transit, mass transit, and the like, depending on the physiological system under investigation. More specifically, as provided herein, the PBPK model is represented by a series of differential equations that describe rate process interactions among anatomical segments for the muti-compartment when a particular drug is nasally administered (i.e., inhaled). The individual compartments are represented mathematically as an integrated compartment kinetic system. In other words, the compartments are linked in a stepwise fashion so as to form an integrated physiological model describing absorption of a compound relative to the anatomical segments.

As provided herein, the PBPK model is defined by the following set of differential equations:

$${V}_{lung} \frac{{dC}_{lung}}{dt} = {Q}_{lung} ( {C}_{ven} - \frac{R {C}_{lung}}{{K}_{pu , lung}} ) - \frac{{f}_{u} {Cl}_{tissue} {RC}_{lung}}{{K}_{pu , lung}}$$

$${V}_{T} \frac{{dC}_{T}}{dt} = {Q}_{T} ( {C}_{art} - \frac{R {C}_{T}}{{K}_{pu , T}} ) - \frac{{f}_{u} {Cl}_{tissue} {RC}_{T}}{{K}_{pu , T}}$$

Where V_{lung} is the volume of the lung tissue, C_{lung} is the concentration of the compound in the lung tissue, Q_{lung} is the blood flow to the lung, Cᵥₑₙ is the concentration of the compound in the venous plasma, R is the blood:plasma ratio, K_{pu,lung} is the tissue:plasma partition coefficient of the compound in the lung, fᵤ is the fraction of the compound unbound in tissue, Clₜᵢₛₛᵤₑ is the clearance rate of the compound from tissue.

Symbols in the second equation have the same meaning where T represents different tissues and organs: adipose, bone, brain, heart, kidneys, muscle, skin, or the rest of the body and Cₐᵣₜ is the concentration of the compound in the arterial plasma. The different tissue and organ compartments are represented by the following equations.

$${V}_{spleen} \frac{{dC}_{spleen}}{dt} = {Q}_{spleen} ( {C}_{art} - \frac{R {C}_{spleen}}{{K}_{pu , spleen}} )$$

$${V}_{gut} \frac{{dC}_{gut}}{dt} = {Q}_{gut} ( {C}_{art} - \frac{R {C}_{gut}}{{K}_{pu , gut}} )$$

$${V}_{liver} \frac{{dC}_{liver}}{dt} = {Q}_{liver} {C}_{art} + {Q}_{spleen} \frac{R {C}_{spleen}}{{K}_{pu , spleen}} + {Q}_{gut} \frac{R {C}_{gut}}{{K}_{pu , gut}} - ( {Q}_{liver} + {Q}_{spleen} + {Q}_{gut} ) \frac{R {C}_{liver}}{{K}_{pu , liver}} - \frac{{f}_{u} {Cl}_{h} {RC}_{liver}}{{K}_{pu , liver}}$$

$${V}_{art} \frac{{dC}_{art}}{di} = {Q}_{lung} ( \frac{R {C}_{lung}}{{K}_{pu , lung}} - {C}_{art} ) - {Cl}_{blood} {C}_{art}$$

$${V}_{ven} \frac{{dC}_{ven}}{di} = \sum {Q}_{T} ( \frac{R {C}_{T}}{{K}_{pu , T}} - {C}_{ven} ) + ( {Q}_{liver} + {Q}_{spleen} + {Q}_{gut} ) ( \frac{R {C}_{liver}}{{K}_{pu , liver}} - {C}_{ven} ) - {Cl}_{blood} {C}_{ven}$$

Where symbols are as above for the compartments denoted in the subscripts. Clₕ is the hepatic clearance rate and Cl_{blood} is the clearance from the blood.

$${V}_{muc , i} \frac{{dC}_{muc , i}}{dt} = - {PA}_{i} ( {C}_{muc , i} {f}_{u , muc} - \frac{{C}_{ep , i}}{{K}_{pu , nose}} ) + {k}_{mcc , in , i} {C}_{muc , i - 1} - {k}_{mcc , out , i} {C}_{muc , i}$$

$${V}_{ep , i} \frac{{dC}_{ep , i}}{dt} = {PA}_{i} ( {C}_{muc , i} {f}_{u , muc} - \frac{{C}_{ep , i}}{{K}_{pu , nose}} ) - {PA}_{i} ( \frac{{C}_{ep , i}}{{K}_{pu , nose}} - \frac{{C}_{sub , i}}{{K}_{pu , nose}} ) - \frac{{f}_{u} {Cl}_{tissue} {RC}_{ep , i}}{{K}_{pu , nose}}$$

$${V}_{sub , i} \frac{{dC}_{sub , i}}{dt} = {PA}_{i} ( \frac{{C}_{sub , i}}{{K}_{pu , nose}} - \frac{{C}_{ep , i}}{{K}_{pu , nose}} ) - {Q}_{noise , i} ( {C}_{art} - \frac{{RC}_{sub , i}}{{K}_{pu , nose}} ) - \frac{{f}_{u} {Cl}_{tissue} {RC}_{sub , i}}{{K}_{pu , nose}}$$

Where V_{muc,i}, C_{muc,i}, V_{ep,i}, C_{ep,i}, V_{sub,i} and C_{sub,i} are the volume and the compound concentration pairs in the nasal mucus, epithelium and sub-epithelium of the ith nasal region (referring to the nasal valve, floor, turbinates, turbinates, olfactory region and rhinopharynx sub-compartments). P is the permeability in the nasal epithelium, Aᵢ is the area of the ith nasal region. k_{mcc,in,i} is the mucociliary clearance rate transporting mass into the ith region (equal to 0 for all regions apart from the rhinopharynx where it equals k_{mcc,ant}) and k_{mcc,out,i} is the mucociliary clearance rate transporting mass out of the ith region (equal to k_{mcc,ant}, which represents the rate of mucociliary clearance pushing material from the nasal floor, turbinates and olfactory region to the rhinopharynx and equal to k_{mcc,post}, which represents the rate of mucociliary clearance pushing mass from the rhinopharynx into the pharynx and eventually gastrointestinal tract).

In some instances, if transport of material from the brain blood into the cerebrospinal fluid (CSF) is being assessed, then the brain compartment is modelled in the system of sub-compartments as outlined in FIG. 3. Specifically, the region beyond the blood brain barrier (BBB) and blood CSF barrier (BCSFB) is separated into brain mass, cranial CSF, and spinal CSF regions. Additionally, as shown in FIG. 4, the transport from the olfactory region of the nasal epithelium to the brain (beyond the BBB and BCSFB) is compartmentalized by showing transport between the nasal area, olfactory, and rhinopharynx sub-compartments. Since the olfactory neuronal cells penetrate the entire depth of the epithelium, transport from both the epithelial and subepithelial sub-compartments of the olfactory region is included in the model. Transport of material from the brain blood into the CSF as outlined by FIGS. 3 and 4 are defined by the following equations:

$${V}_{muc , i} \frac{{dC}_{muc , i}}{dt} = - {PA}_{i} ( {C}_{muc , i} {f}_{u , muc} - {K}_{pu , nose} {C}_{ep , i} ) + {k}_{mcc , in , i} {C}_{muc , i - 1} - {k}_{mcc , out , i} {C}_{muc , i}$$

$${V}_{ep , i} \frac{{dC}_{ep , i}}{dt} = {PA}_{i} ( {C}_{muc , i} {f}_{u , muc} - {K}_{pu , nose} {C}_{ep , i} ) - {PA}_{i} ( {K}_{pu , nose} {C}_{ep , i} - {K}_{pu , nose} {C}_{sub , i} ) - \frac{{f}_{u} {Cl}_{tissue} {RC}_{ep , i}}{{K}_{pu , nose}} - {k}_{brain , in} {K}_{pu , nose} {C}_{ep , i} + {k}_{brain , out} {f}_{u , ccsf} {C}_{ccsf}$$

$${V}_{sub , i} \frac{{dC}_{sub , i}}{dt} = {PA}_{i} ( {K}_{pu , nose} {C}_{ep , i} - {K}_{pu , nose} {C}_{sub , i} ) - {Q}_{noise , i} ( {C}_{art} - \frac{{RC}_{sub , i}}{{K}_{pu , nose}} ) - \frac{{f}_{u} {Cl}_{tissue} {RC}_{sub , i}}{{K}_{pu , nose}} - {k}_{brain , in} {K}_{pu , nose} {C}_{sub , i} + {k}_{brain , out} {f}_{u , ccsf} {C}_{ccsf}$$

Where V_{muc,i}, C_{muc,i}, V_{ep,i}, C_{ep,i}, V_{sub,i} and C_{sub,i} are the volume and drug concentration pairs in the nasal mucus, epithelium and sub-epithelium of the ith nasal region (referring to the nasal valve, floor, turbinates, olfactory region and rhinopharynx sub-compartments). P is the permeability in the nasal epithelium, Aᵢ is the area of the ith nasal region. k_{mcc,in,i} is the mucociliary clearance rate transporting mass into the ith region (equal to 0 for all regions apart from the rhinopharynx where it equals k_{mcc,ant}) and k_{mcc,out,i} is the mucociliary clearance rate transporting mass out of the ith region (equal to k_{mcc,ant} for the nasal floor, turbinates, and olfactory region and equal to k_{mcc,post} for the rhinopharynx). k_{brain,in} is the rate of transport from the olfactory tissue to the cerebrospinal fluid via the intracellular and extracellular pathways, k_{brain,out} is the rate of transport from the cerebrospinal fluid to the olfactory tissue via the intracellular and extracellular pathways (these two parameters are zero for the non-olfactory regions). f_{u,ccsf} is the fraction of the drug unbound in the cranial CSF and C_{ccsf} is the concentration in the cranial CSF.

$${V}_{brain} \frac{{dC}_{brain}}{dt} = {Q}_{brain} ( {C}_{art} - {RC}_{brain} ) - {f}_{u} {Cl}_{tissue} {RC}_{brain} + {P}_{b} {A}_{b} ( {f}_{u , bm} {C}_{bm} - {f}_{u , brain} {C}_{brain} ) {P}_{c} {A}_{c} ( {f}_{u , ccsf} {C}_{ccsf} - {f}_{u , brain} {C}_{brain} ) + {Q}_{ssink} {C}_{scsf} + {Q}_{csink} {C}_{ccsf}$$

$${V}_{bm} \frac{{dC}_{bm}}{dt} = {P}_{b} {A}_{b} ( {f}_{u , brain} {C}_{brain} - {f}_{u , bm} {C}_{bm} ) + {P}_{e} {A}_{e} ( {f}_{u , ccsf} {C}_{ccsf} - {f}_{u , bm} {C}_{bm} ) - {Q}_{bulk} {C}_{bm}$$

$${V}_{ccsf} \frac{{dC}_{ccsf}}{dt} = {P}_{e} {A}_{e} ( {f}_{u , bm} {C}_{bm} - {f}_{u , ccsf} {C}_{ccsf} ) + {P}_{c} {A}_{c} ( {f}_{u , brain} {C}_{brain} - {f}_{u , ccsf} {C}_{ccsf} ) + {Q}_{bulk} {C}_{bm} + {Q}_{sout} {C}_{scsf} - {Q}_{sin} {C}_{ccsf} - {Q}_{csink} {C}_{ccsf} + {k}_{brain , in} {K}_{pu , nose} {C}_{ep , i} - {k}_{brain , out} {f}_{u , ccsf} {C}_{ccsf} + {k}_{brain , in} {K}_{pu , nose} {C}_{sub , i} - {k}_{brain , out} {f}_{u , ccsf} {C}_{ccsf}$$

$${V}_{scsf} \frac{{dC}_{scsf}}{dt} = {Q}_{sin} {C}_{ccsf} - {Q}_{sout} {C}_{scsf} - {Q}_{ssink} {C}_{scsf}$$

Where V_{brain} is the volume of the brain blood, C_{brain} is the concentration of the drug molecule in the brain blood, Q_{brain} is the blood flow to the brain, P_{b}A_{b} is the product of the permeability and surface area between brain blood and brain mass, f_{u,bm} is the fraction of the drug unbound in the brain mass, C_{bm} is the concentration of blood in the brain mass, f_{u,brain} is the fraction of the drug unbound in the brain blood, P_{c}A_{c} is the product of the permeability and surface area between brain blood and cranial, f_{u,ccsf} is the fraction of the drug unbound in the cranial CSF, C_{ccsf} is the concentration of the drug in the cranial CSF, Q_{csink} and Qₛₛᵢₙₖ are the flows from the cranial and spinal CSF to the brain blood respectively, C_{scsf} is the concentration of the drug in the spinal CSF, V_{bm} is the volume of the brain mass, PₑAₑ is the product of the permeability and surface area between brain mass and cranial CSF, Q_{bulk} is the bulk flow from the brain mass to the cranial CSF and Qₛₒᵤₜ and Qₛᵢₙ are the CSF shuttle flow between the cranial and spinal CSF.

b. Parameterization of the PBPK Model

PBPK models are useful in part because of their use of two key components, that is, compound-specific parameters (e.g., partition coefficients and metabolic rates) and species-specific physiological or anatomical parameters (e.g., cardiac output, organ weights, blood flow rates, relevant concentrations, etc.). Once the PBPK model, updated with the brain and nose compartments and sub-compartments as discussed herein is prepared, one must fill in the various physiological-specific and compound-specific parameters. In this step, all the parameters describing the compartments and sub-compartments of the PBPK model that are involved in any one of the differential equations must be provided so that the model will function.

i. Physiological-Specific Parameters

The use of accurate species-specific physiological parameters is important in the development, validation, extrapolation, and application of a PBPK model. These parameters are well known for many species and can be obtained from the literature and database searching. Examples of physiological parameters (and their values) that can be implemented into the present PBPK model are listed in Table 1 below. The inclusion of a specific physiological parameter is dependent on the compartments (i.e., organs/tissues) that are included in the model.

TABLE 1
Physiological-Specific Parameters

| Parameter | Value | Source |
|---|---|---|
| Q_{CO} - Cardiac output | 5200.0 mL/min | R. P. Brown, M. D. |
| | | Delp, S. L. |
| | | Lindstedt, L. R. |
| | | Rhomberg and R. |
| | | P. Beliles, |
| | | “Physiological |
| | | parameter values |
| | | for physiologically |
| | | based |
| | | pharmacokinetic |
| | | models.,” |
| | | Toxicology and |
| | | Industrial Health, |
| | | vol. 13, pp. 407- |
| | | 484, 1997 and |
| | | references within |
| Qₙₒₛₑ - Blood flow to | 0.003Q_{co} | Notes for Table 1 in |
| nose | | M. S. Bogdanffy, S. |
| | | R. D. R. Plowchalk, |
| | | A. Jarabek and M. |
| | | E. Andersen, “A |
| | | biologically-based |
| | | risk assessment for |
| | | vinyl acetate- |
| | | induced cancer and |
| | | non-cancer |
| | | inhalation toxicity,” |
| | | Toxicological |
| | | Sciences, vol. 51, |
| | | pp. 19-35, 1999 and |
| | | references within |
| Q_{adipose} - Blood flow | 0.052Q_{co} | Table 27 in R. P. |
| to adipose | | Brown, M. D. Delp, |
| | | S. L. Lindstedt, L. |
| | | R. Rhomberg and |
| | | R. P. Beliles, |
| | | “Physiological |
| | | parameter values |
| | | for physiologically |
| | | based |
| | | pharmacokinetic |
| | | models.,” |
| | | Toxicology and |
| | | Industrial Health, |
| | | vol. 13, pp. 407- |
| | | 484, 1997 and |
| | | references within |
| Q_{bone} - Blood flow to | 0.042Q_{co} | Same as Q_{adipose} |
| Q_{brain} - Blood flow to | 0.114Q_{co} | Same as Q_{adipose} |
| brain | | |
| Qₕₑₐᵣₜ - Blood flow to | 0.4Q_{co} | Same as Q_{adipose} |
| heart | | |
| Q_{kidney}- Blood flow | 0.175Q_{co} | Same as Q_{adipose} |
| to kidneys | | |
| Q_{muscle} - Blood flow | 0.191Q_{co} | Same as Q_{adipose} |
| to muscle | | |
| Qₛₖᵢₙ - Blood flow to | 0.058Q_{co} | Same as Q_{adipose} |
| skin | | |
| Q_{gut} - Blood flow to | 0.17Q_{co} | Table 5 in O. |
| gut | | Luttringer, F .- P. |
| | | Thiel, P. Poulin, A. |
| | | H. Schmitt- |
| | | Hoffmann, T. W. |
| | | Guentert and T. |
| | | Lavé, |
| | | “Physiologically |
| | | Based |
| | | Pharmacokinetic |
| | | (PBPK) Modeling |
| | | of Disposition of |
| | | Epiroprim in |
| | | Humans,” Journal |
| | | of Pharmaceutical |
| | | Sciences, vol. 92, |
| | | pp. 1990-2007, |
| | | 2003 and references |
| | | within |
| Qₛₚₗₑₑₙ - Blood flow | 0.02Q_{co} | Same as Q_{gut} |
| to spleen | | |
| Qₗᵢᵥₑᵣ - Blood flow to | 0.227Q_{co} | Same as Q_{adipose} |
| liver | | |
| Qrest - Blood flow to the rest of the body | ${Q}_{CO} - {\sum }_{i \neq lung} {Q}_{i}$ | — |
| W - Body weight | 70 kg | International |
| | | Commission on |
| | | Radiological |
| | | Protection, “Human |
| | | Respiratory Tract |
| | | Model for |
| | | Radiological |
| | | Protection,” 1994 |
| ρ_{T} - Tissue density | 1.00 g mL⁻¹ | Table 19 and |
| (apart from bone) | | associated |
| | | discussion in R. P. |
| | | Brown, M. D. Delp, |
| | | S. L. Lindstedt, L. |
| | | R. Rhomberg and |
| | | R. P. Beliles, |
| | | “Physiological |
| | | parameter values |
| | | for physiologically |
| | | based |
| | | pharmacokinetic |
| | | models.,” |
| | | Toxicology and |
| | | Industrial Health, |
| | | vol. 13, pp. 407- |
| | | 484, 1997 |
| ρ_{bone} - Bone density | 1.99 g mL⁻¹ | R. P. Brown, M. D. |
| | | Delp, S. L. |
| | | Lindstedt, L. R. |
| | | Rhomberg and R. |
| | | P. Beliles, |
| | | “Physiological |
| | | parameter values |
| | | for physiologically |
| | | based |
| | | pharmacokinetic |
| | | models.,” |
| | | Toxicology and |
| | | Industrial Health, |
| | | vol. 13, pp. 407- |
| | | 484, 1997, and |
| | | references within. |
| V_{lung} - Volume of | 0.0076W/ρ_{T} | Table 7 in R. P. |
| lung tissue | | Brown, M. D. Delp, |
| | | S. L. Lindstedt, L. |
| | | R. Rhomberg and |
| | | R. P. Beliles, |
| | | “Physiological |
| | | parameter values |
| | | for physiologically |
| | | 0.0076W/PT |
| | | based |
| | | pharmacokinetic |
| | | models.,” |
| | | Toxicology and |
| | | Industrial Health, |
| | | vol. 13, pp. 407- |
| | | 484, 1997 and |
| | | references within |
| V_{adipose} - Volume of | 0.2142W/ρ_{T} | Same as V_{lung} |
| adipose | | |
| V_{bone} - Volume of | 0.1429W/ρ_{bone} | Same as V_{lung} |
| bone | | |
| V_{brain,total} - Total | 0.0047W/ρ_{T} | Same as V_{lung} |
| volume of brain tissue | | |
| Vₕₑₐᵣₜ - Volume of | 0.0047W/ρ_{T} | Same as V_{lung} |
| heart tissue | | |
| V_{kidney} - Volume of | 0.0044W/ρ_{T} | Same as V_{lung} |
| kidney tissue | | |
| V_{muscle} - Volume of | 0.4W/ρ_{T} | Same as V_{lung} |
| muscle tissue | | |
| Vₛₖᵢₙ - Volume of skin | 0.0371W/ρ_{T} | Same as V_{lung} |
| tissue | | |
| V_{gut} - Volume of gut | 0.0171W/ρ_{T} | Same as V_{lung} |
| tissue | | |
| Vₛₚₗₑₑₙ - Volume of | 0.0026W/ρ_{T} | Same as V_{lung} |
| spleen tissue | | |
| Vₗᵢᵥₑᵣ - Volume of | 0.0257W/ρ_{T} | Same as V_{lung} |
| liver tissue | | |
| Vₐᵣₜ - Volume of | 0.05925W/ρ_{T} | Table 21 and |
| arterial blood | | associated |
| | | discussion on page |
| | | 54 of R. P. Brown, |
| | | M. D. Delp, S. L. |
| | | Lindstedt, L. R. |
| | | Rhomberg and R. |
| | | P. Beliles, |
| | | “Physiological |
| | | parameter values |
| | | for physiologically |
| | | based |
| | | pharmacokinetic |
| | | models.,” |
| | | Toxicology and |
| | | Industrial Health, |
| | | vol. 13, pp. 407- |
| | | 484, 1997 |
| Vᵥₑₙ - Volume of | 0.01975W/ρ_{T} | Same as Vₐᵣₜ |
| venous blood | | |
| Vᵣₑₛₜ - Volume of tissue in the rest of the body | $( W - {ρ}_{T} {\sum }_{i \neq bone} ( {V}_{i} ) - {ρ}_{bone} {V}_{bone} ) / {ρ}_{T}$ | — |
| k_{mcc,ant} - Anterior | 0.0625 hr⁻¹ | I. Gonda and E. |
| mucociliary clearance | | Gipps, “Model of |
| rate | | Disposition of |
| | | Drugs |
| | | Administered into |
| | | the Human Nasal |
| | | Cavity,” Pharm |
| | | Res, vol. 7, pp. 69- |
| | | 75, 1990 |
| k_{mcc,post} - Posterior | 4.167 hr⁻¹ | Same as k_{mcc,ant} |
| mucociliary clearance | | |
| rate | | |
| Aᵥₐₗᵥₑ - Area of the | 40128 mm² | Internal Pharma |
| nasal valve | | data |
| A_{floor} - Area of the | 4019 mm² | Internal Pharma |
| nasal floor | | data |
| A_{turb} - Area of the | 9057 mm² | Internal Pharma |
| turbinates | | data |
| A_{olf} - Area of the | 9743 mm² | Internal Pharma |
| olfactory region | | data |
| Aᵣₕᵢₙₒ - Area of the | 2919 mm² | Internal Pharma |
| rhinopharynx | | data |
| Qᵥₐₗᵥₑ - Blood flow to | 0 | Estimated |
| the nasal valve | | |
| Q_{floor} - Blood flow to the nasal floor | $0.5 \frac{{Q}_{nose} {A}_{floor}}{{A}_{floor} + {A}_{turb} + {A}_{rhino}}$ | Following method outlined in M. S. Bogdanffy, S. R. D. |
| | | R. Plowchalk, A. |
| | | Jarabek and M. E. |
| | | Andersen, “A |
| | | biologically-based |
| | | risk assessment for |
| | | vinyl acetate- |
| | | induced cancer and |
| | | non-cancer |
| | | inhalation toxicity,” |
| | | Toxicological |
| | | Sciences, vol. 51, |
| | | pp. 19-35, 1999 |
| Q_{turb} - Blood flow to the turbinates | $0.5 \frac{{Q}_{nose} {A}_{turb}}{{A}_{floor} + {A}_{turb} + {A}_{rhino}}$ | Same as Q_{floor} |
| Q_{olf} - Blood flow to | 0.5Qₙₒₛₑ | Same as Q_{floor} |
| the olfactory region | | |
| Qᵣₕᵢₙₒ - Blood flow to the rhinopharynx | $0.5 \frac{{Q}_{nose} {A}_{rhino}}{{A}_{floor} + {A}_{turb} + {A}_{rhino}}$ | Same as Q_{floor} |
| T_{valve,muc} - Thickness | 10 μm | C. B. Frederick, M. |
| of mucus in the nasal | | L. Bush, L. G. |
| valve | | Lomax, K. A. |
| | | Black, L. Finch, J. |
| | | S. Kimbell, K. T. |
| | | Morgan, R. P. |
| | | Subramaniam, J. B. |
| | | Morris and J. S. |
| | | Ultman, |
| | | “Application of a |
| | | hybrid |
| | | computational fluid |
| | | dynamics and |
| | | physiologically |
| | | based inhalation |
| | | model for |
| | | interspecies |
| | | dosimetry |
| | | extrapolation of |
| | | acidic vapors in the |
| | | upper airways,” |
| | | Toxicology and |
| | | Applied |
| | | Pharmacology, vol. |
| | | 152, pp. 211-231, |
| | | 1998 |
| T_{floor,muc} - Thickness | 10 μm | Same as T_{valve,muc} |
| of mucus in the nasal | | |
| floor | | |
| T_{turb,muc} - Thickness | 10 μm | Same as T_{valve,muc} |
| of mucus in the | | |
| turbinates | | |
| T_{olf,muc} - Thickness of | 20 μm | Same as T_{valve,muc} |
| mucus in the | | |
| olfactory region | | |
| T_{rhino,muc} - Thickness | 10 μm | Same as T_{valve,muc} |
| of mucus in the | | |
| rhinopharynx | | |
| T_{valve,ep} - Thickness | 25 μm | Same as T_{valve,muc} |
| of epithelium in the | | |
| nasal valve | | |
| T_{floor,ep} - Thickness | 25 μm | Same as T_{valve,muc} |
| of mucus in the nasal | | |
| floor | | |
| T_{turb,ep} - Thickness of | 25 μm | Same as T_{valve,muc} |
| mucus in the | | |
| turbinates | | |
| T_{olf,ep} - Thickness of | 80 μm | Same as T_{valve,muc} |
| epithelium in the | | |
| olfactory region | | |
| T_{rhino,ep} - Thickness | 25 μm | Same as T_{valve,muc} |
| of epithelium in the | | |
| rhinopharynx | | |
| T_{valve,sub} - Thickness | 75 μm | Same as T_{valve,muc} |
| of subepithelium in | | |
| the nasal valve | | |
| T_{floor,sub} - Thickness | 75 μm | Same as T_{valve,muc} |
| of subepithelium in | | |
| the nasal floor | | |
| T_{turb,sub} - Thickness | 75 μm | Same as T_{valve,muc} |
| of mucus in the | | |
| turbinates | | |
| T_{olfsub} - Thickness of | 50 μm | Same as T_{valve,muc} |
| subepithelium in the | | |
| olfactory region | | |
| T_{rhino,sub} - Thickness | 75 μm | Same as T_{valve,muc} |
| of subepithelium in | | |
| the rhinopharynx | | |
| V_{brain} - Volume of | 0.05V_{brain,tot} | Supplementary |
| brain blood | | information of L. F. |
| | | Verscheijden, J. B. |
| | | Koenderink, S. N. |
| | | de Wildt and F. G. |
| | | Russel, |
| | | “Development of a |
| | | physiologically-based |
| | | pharmacokinetic |
| | | pediatric brain |
| | | model for |
| | | prediction of |
| | | cerebrospinal fluid |
| | | drug concentrations |
| | | and the influence of |
| | | meningitis,” PLoS |
| | | Computational |
| | | Biology, vol. 15, p. |
| | | e1007117, 2019 |
| | | and references within |
| V_{brain,endothelial} - | 0.005V_{brain,tot} | Same as V_{brain} |
| Volume of brain | | |
| endothelium | | |
| V_{bm} - Volume of | V_{brain,tot} − brain,endothelial − | Same as V_{brain} |
| brain mass | V_{brain} − V_{ccsf} − V_{scsf} | |
| V_{ccsf} - Volume of | (0.105 × 0.8)V_{brain,tot} | Same as V_{brain} |
| cranial CSF | | |
| V_{scsf} - Volume of | (0.105 × 0.2)V_{brain,tot} | Same as V_{brain} |
| spinal CSF | | |
| Qₛₛᵢₙₖ - Flow from the | 0.38(0.75Q_{productionrate} + Q_{bulk}) | Same as V_{brain} |
| spinal CSF to the | | |
| brain blood | | |
| Q_{csink} - Flow from the | 0.75Q_{productionrate} + Q_{bulk} − Qₛᵢₙ + Qₛₒᵤₜ | Same as V_{brain} |
| cranial CSF to the | | |
| brain blood | | |
| Q_{bulk} - Bulk flow | 0.25Q_{productionrate} | Same as V_{brain} |
| from the brain mass to | | |
| the cranial CSF | | |
| Qₛᵢₙ - shuttle flow | Qₛₛᵢₙₖ + Qₛₒᵤₜ | Same as V_{brain} |
| from the spinal to | | |
| cranial CSF | | |
| Qₛₒᵤₜ - shuttle flow | 0.9Qₛₛᵢₙₖ | Same as V_{brain} |
| from the cranial to | | |
| spinal CSF | | |
| Q_{productionrate} - CSF | 0.021 | Same as V_{brain} |
| production rate | | |

ii. Compound-Specific Parameters

Compound-specific parameters are characteristics that have been obtained for a specific compound (i.e., drug, chemical entity) from any type of pre-clinical studies. For example, this could include in vitro cell assays, in vitro protein assays (e.g., binding data), and/or in vivo studies done in mice, rat, or any other laboratory animal.

When building the PBPK model, known drugs with known compound-specific parameters (pre-clinical and clinical) are used to test and train the model.

Examples of compound-specific parameters that can be implemented into the present PBPK model include:

TABLE 2
Compound-Specific Parameters

Kₚᵤ, _{adipose} - Tissue: plasma partition coefficient for the lung
Kₚᵤ, _{bone} - Tissue: plasma partition coefficient for the bone
Kₚᵤ, _{brain} - Tissue: plasma partition coefficient for the brain
Kₚᵤ, ₕₑₐᵣₜ - Tissue: plasma partition coefficient for the heart
Kₚᵤ, _{kidney} - Tissue: plasma partition coefficient for the kidney
Kₚᵤ, _{muscle} - Tissue: plasma partition coefficient for the muscle
Kₚᵤ, ₛₖᵢₙ - Tissue: plasma partition coefficient for the skin
Kₚᵤ, _{gut} - Tissue: plasma partition coefficient for the gut
Kₚᵤ, ₛₚₗₑₑₙ - Tissue: plasma partition coefficient for the spleen
Kₚᵤ, ₗᵢᵥₑᵣ - Tissue: plasma partition coefficient for the liver
Kₚᵤ, _{lung} - Tissue: plasma partition coefficient for the lung
Kₚᵤ, ₙₒₛₑ - Tissue: plasma partition coefficient for the nose
Kₚᵤ, ᵣₑₛₜ- Tissue: plasma partition coefficient for the rest of the body
pKa - pH at which concentrations of ionized and un-ionized forms
of compound are equal
LogP - drug partition coefficient between octanol and water
fᵤ - Fraction of unbound compound in blood plasma
Clₕ - Hepatic Clearance
P - Permeability in the nasal epithelium
R - Blood/plasma ratio
f_{K}_{ₚᵤ}- Factor by which all Kₚᵤ are scaled to improve model
agreement with clinical data
Cl_{blood} - Blood clearance of compound

iii. Necessary Parameters for PBPK Model

For the PBPK model to work properly for a new compound to be analyzed and modeled, the following compound-specific parameters are required as inputs:
P—Permeability in the nasal epithelium,
   approximated using RPMI 2650, Calu-3, or other cell line models;
Log P—Log of the octanol/water partition coefficient (used to calculate the blood:tissue partition coefficients using common methods);
pKa—Negative log of the acid dissociation constant (used to calculate the blood:tissue partition coefficients);
fu—fraction of drug dissolved in blood plasma that is unbound to plasma proteins,
   measured using protein binding assays;
R—blood/plasma ratio, ratio of drug dissolved in the entire blood to that dissolved in the blood plasma,
   measured by dissolving the drug in a sample of blood and separating the plasma;
Clₕ—Hepatic clearance (clearance in the liver),
   meausured using hepatic or mitochondrial clearance assays,
   typically adjusted to improve the model agreement with clinical pharmacokinetic data (once available);
Mass of the compound deposited in each region of the nose,
   Measured using a nasal cast.

### B. Nose-to-Brain PBPK Model Application

Also provided herein are methods for predicting delivery and pharmacokinetics of a new or previously unknown, candidate nasally administered (i.e., inhaled) drug to the brain tissue by using the PBPK model as described herein.

### Pre-Clinical Data

The nose-to-brain PBPK model is updated with pre-clinical data known about the compound, as discussed in detail above in the sub-section “Compound-Specific Parameters.” Specifically, compound-specific parameters are characteristics that have been obtained for the compound from pre-clinical studies. For example, this could include in vitro cell-based assays, in vitro protein assays (e.g., binding data), and/or in vivo studies done in mice, rat, or any other laboratory animal.

### Nasal Deposition Data

The nose-to-brain model is then updated with nasal cast deposition data. Nasal cast deposition data can be obtained from any known in vitro nasal cast devices or physical models. Briefly, nasal casts help facilitate formulation and product development. Nasal deposition has been shown to be linked to pharmacokinetic outcomes. Nasal casts, which are replicas of the human nasal cavity, have evolved from models made from cadavers to complex 3D printed replicas. They can be segmented into regions of interest for quantification of deposition and different techniques have been utilized to quantify deposition.

In some instances of the presently described methods, the Aeronose® nasal cast from AptarGroup, Inc. (Crystal Lake, IL) was utilized. The nasal casts can be tested with various drug delivery devices in the form of nasal spray devices and nasal inhalation devices, including, for example, Adella N2B (D03/L06), CPS, Preloaded Nozzle, UDS1 Adella N2B, UDS1 std, UDSp Liquide, VP7+232 N2B, VP7+CB18.

The drug is sprayed (via a nasal spray device) onto the nasal cast and the mass of the drug that is deposited in each region of the nasal cast is entered into the model.

### Running the Model

Once the necessary inputs (physiological parameters, compound parameters, nasal deposition data, and time points that are to be solved) are entered into the model, the mathematical equations describing the model (see “Compartments” section) are solved via numerical methods using any suitable programing software (e.g., SciPy, MATLAB, DifferentialEquations.j1).

### Prediction

Once the model is run, a report is produced that comprises information relating to the concentration of the drug in each compartment at one or more time points. This information can then be further analyzed (i.e., to obtain pharmacokinetic data) to provide a prediction of delivery results for the drug to brain tissue via nasal inhalation.

### Model Refinement

In order to determine appropriate values for the parameters of the model, available clinical data describing possible evolutions of the models are used (i.e., intravenous data and/or nasal data). This data is used to further refine the model. The model prediction of concentration in one or more model compartments representing regions of the body (i.e. venous or arterial blood plasma, cerebrospinal fluid, nasal tissue or brain tissue) at the clinical time points is compared to the clinical concentrations. Optimization of model parameters is then achieved by any suitable method. It can be performed for instance by minimizing an objective function (i.e. least squares or average fold error) using a suitable algorithm (e.g. Levenberg-Marquardt or sequential least squares programming). The subset of parameters whose values are already known may remain fixed (e.g. subject body weight) and other parameters will be allowed to vary within bounds that are informed by either best practice or prior knowledge (i.e. known ranges for the class of molecule being modelled or variability in pre-clinical data). In ideal cases intravenous and nasally-administered clinical data are used where a first step is conducted using intravenous data to optimize parameters related to systemic distribution (i.e. those related to tissue:plasma partition coefficients, blood-brain-barrier permeability and/or clearance), these are then fixed and a second step is conducted to optimize parameters related to transport from the nasal cavity (i.e. epithelial permeability and/or transport rates along the olfactory or trigeminal nerves).

In some embodiments, if pharmacokinetic data following intravenous administration is available, this can be used to refine the systemic disposition parameters (tissue:plasma partition coefficients, hepatic and blood clearance). If the initial elimination phase (just after maximum concentration) is too slow then the hepatic clearance is increased (or vice versa). If the terminal elimination phase is too slow then the tissue:plasma partition coefficients can be scaled down (these are typically all scaled down by the same amount) or vice versa. If the whole curve is too low, then then blood clearance can be introduced, increasing blood clearance to move the predicted concentrations down and vice versa. The adjustments are typically one parameter at a time in large strides of ~10% to find the correct region and then adjusted in smaller increments to refine the parameter. Once all parameters have been optimized by eye, goodness of fit metrics like R², chi square or sum of squares can be used to fine-tune the parameters further.

Similarly, if pharmacokinetic data following nasal administration is available, this can be used to refine the nasal permeability. This is adjusted in the same way as described above with respect to intravenous data.

### C. PBPK Model Systems

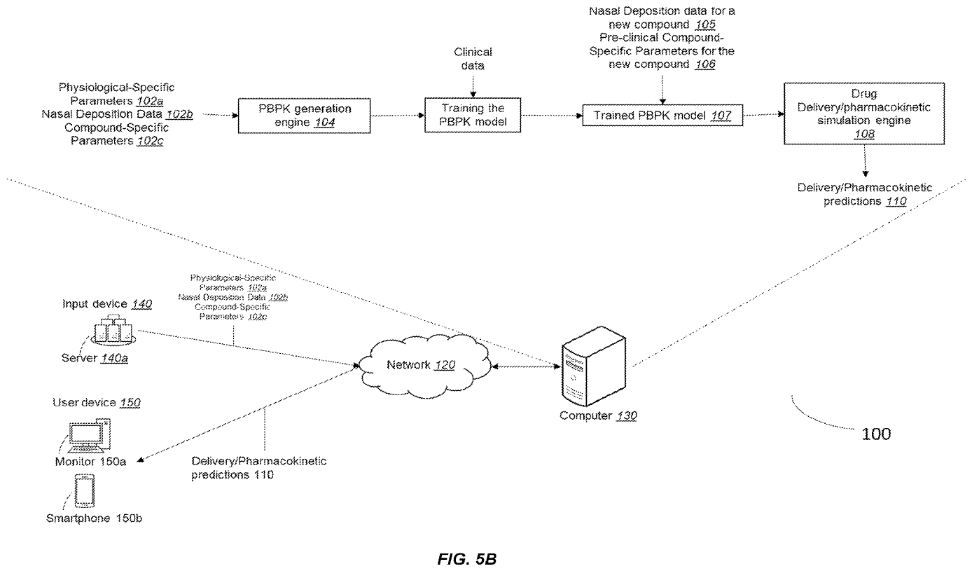

The PBPK models can be generated, trained, and used to model results for new drug compounds in various computer and/or hardware, or hybrid, systems. Here we disclose one example of a computer and/or hardware system to generate such a physiologically-based pharmacokinetic model. FIG. 5A is a flow chart showing the steps for generating, training, and using the presently described PBPK models. An initial PBPK model is generated by defining the compartments as outlined in FIGS. 1-4 (10a). Additionally, the differential equations describing the relationships between the compartments are defined (10b). The physiological parameters specific for a particular species for each compartment and sub-compartment are defined (10c). The compound-specific parameters for a known compound (that has known pharmacokinetic data) for one or more, or all, of the compartments and one or more, or all, of the sub-compartments are also defined (10d).

Then the model is parameterized by including parameters in the initial nose-to-brain PBPK model to include data for a plurality of physiological parameters for one or more, or all, of the compartments and/or one or more, or all, of the sub-compartments (20a). It also includes parameterizing the initial nose-to-brain PBPK model to include compound-specific parameters for a drug with known pharmacokinetic data for one or more, or all, of the compartments and one or more, or all, of the sub-compartments, to generate a parameterized nose-to-brain PBPK model (20a). The compound specific parameters can be obtained from pre-clinical studies. Then, the parameterized nose-to-brain model is run by solving the equations using an engine that has a differential equation solver (20b).

Next, a report is then generated by running the parameterized nose-to-brain model, which includes drug concentration data in one or more of the compartments and/or one or more of the sub-compartments at one or more time points (30). Finally, the drug concentration data in the report is analyzed and compared with known pharmacokinetic data for the compound (40). The parameterized nose-to-brain model is then updated to obtain a trained nose-to-brain model (40). Once the trained nose-to-brain model is built, predictions for a new compound with unknown pharmacokinetic data can be simulated using the model (50).

FIG. 5B is a block diagram of an example of a system 100 for generating and using a PBPK model to predict delivery of a nasally administered drug to the brain. The system 100 includes an input device 140, a network 120, and one or more computers 130 (e.g., one or more local or cloud-based processors).

The input device 140 is configured to input physiological-specific parameters 102a, nasal deposition data 102b, and compound-specific parameters 102c and provide the physiological-specific parameters 102a, nasal deposition data 102b, and compound-specific parameters 102c to another device across a network 120. The physiological-specific parameters 102a include the parameters listed, for example, in Table 1. They are species-specific and are obtained through literature and data searches. The nasal deposition data 102b includes the deposition data obtained after a drug has been sprayed into a nasal cast. The amount (e.g., mass) of the compound in each region of the nasal cast is the nasal deposition data 102c. The compound-specific parameters 102c include pre-clinical data (and sometimes clinical data). For example, this could include in vitro cell-based assays, in vitro protein assays (e.g., binding data), and/or in vivo studies done in mice, rat, or any other laboratory animal.

In some embodiments, the input device 140 can include a server 140a that is configured to obtain the physiological-specific parameters 102a and compound-specific parameters 102c from literature searches or relevant pharmacology-related databases. In some implementations, the one or more other input devices can access and also receive the nasal deposition data 102b and transmit the nasal deposition data 102b to the computer 130 via the network 120. The network 120 represents a computer network and can include one or more of a wired Ethernet network, a wired optical network, a wireless WiFi network, a LAN, a WAN, a Bluetooth network, a cellular network, the Internet, or other suitable network, or any combination thereof.

The computer 130 is configured to obtain and store the physiological-specific parameters 102a, nasal deposition data 102b, and compound-specific parameters 102c and generate a PBPK model. The computer 130 can include the necessary engines to write and/or access the necessary software that can be used for generating the differential equations defining the PBPK model and a solver for solving the differential equations. In some implementations, the computer 130 is a server. For purposes of the present disclosure, an “engine” can include one or more software modules, one or more hardware modules, or a combination of one or more software modules and one or more hardware modules. In some implementations, one or more computers are dedicated to a particular engine. In some implementations, multiple engines can be installed and running on the same computer or computers.

The model generation engine 104 is configured to define the separate compartments and sub-compartments of a PBPK model (see e.g., FIGS. 1-4). Once the model has been generated by model generation engine 104, a simulation is run for a known compound with known pharmacokinetic data. The model is updated and trained by comparing the simulations against known clinical data for the compound.

To obtain pharmacokinetic data for a new compound, nasal deposition data for a new compound 105 and pre-clinical compound-specific parameters for the new compound 106 are obtained and entered into the updated/trained PBPK model 107. A simulation engine 108 can then provide delivery/pharmacokinetic predictions 110 for the specific new compound. The computer 130 can generate rendering data that, when rendered by a device having a display such as a user device 150 (e.g., a computer having a monitor 150a, a mobile computing device such as a smart phone 150b, or another suitable user device).

### D. Systems for Building and Using the PBPK Models

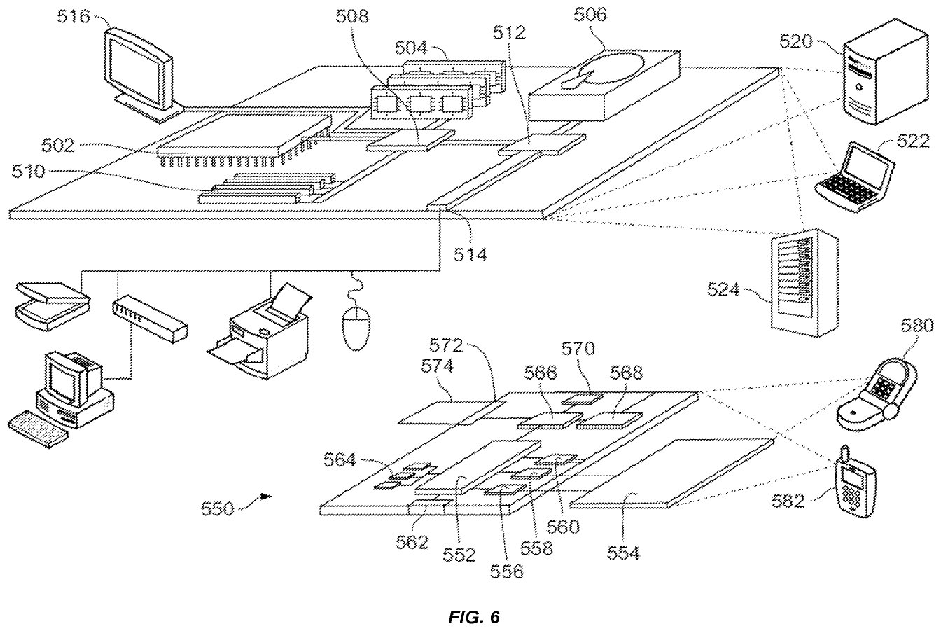

FIG. 6 illustrates an example of a block diagram of system components that can be used to implement systems, models, and methods described herein. FIG. 6 shows a computing device 500 that represents any one or more of various forms of digital computers, such as laptops, desktops, workstations, personal digital assistants, servers, blade servers, mainframes, and other appropriate computers. Computing device 550 is intended to represent various forms of mobile devices, such as personal digital assistants, cellular telephones, smartphones, and other similar computing devices. Additionally, computing device 500 or 550 can include Universal Serial Bus (USB) flash drives. The USB flash drives can store operating systems and other applications. The USB flash drives can include input/output components, such as a wireless transmitter or USB connector that can be inserted into a USB port of another computing device. The components shown here, their connections and relationships, and their functions, are meant to be exemplary only, and are not meant to limit implementations of the inventions described and/or claimed in this document.

Computing device 500 includes a processor 502, memory 504, a storage device 506, a high-speed controller 508 connecting to memory 504 and high-speed expansion ports 510, and a low speed controller 512 connecting to low speed bus 514 and storage device 506. Each of the components 502, 504, 508, 508, 510, and 512, are interconnected using various busses, and can be mounted on a common motherboard or in other manners as appropriate. The processor 502 can process instructions for execution within the computing device 500, including instructions stored in the memory 504 or on the storage device 506 to display graphical information for a GUI on an external input/output device, such as display 516 coupled to high speed controller 508. In other implementations, multiple processors and/or multiple buses can be used, as appropriate, along with multiple memories and types of memory. Also, multiple computing devices 500 can be connected, with each device providing portions of the necessary operations, e.g., as a server bank, a group of blade servers, or a multi-processor system.

The memory 504 stores information within the computing device 500. In one implementation, the memory 504 is a volatile memory unit or units. In another implementation, the memory 504 is a non-volatile memory unit or units. The memory 504 can also be another form of computer-readable medium, such as a magnetic or optical disk.

The storage device 506 is capable of providing mass storage for the computing device 500. In one implementation, the storage device 506 can be or contain a computer-readable medium, such as a floppy disk device, a hard disk device, an optical disk device, or a tape device, a flash memory or other similar solid-state memory device, or an array of devices, including devices in a storage area network or other configurations. A computer program product can be tangibly embodied in an information carrier. The computer program product can also contain instructions that, when executed, perform one or more methods, such as those described above. The information carrier is a computer- or machine-readable medium, such as the memory 504, the storage device 506, or memory on processor 502.

The high-speed controller 508 manages bandwidth-intensive operations for the computing device 500, while the low speed controller 512 manages lower bandwidth intensive operations. Such allocation of functions is an example only. In one implementation, the high-speed controller 508 is coupled to memory 504, display 516, e.g., through a graphics processor or accelerator, and to high-speed expansion ports 510, which can accept various expansion cards (not shown). In the implementation, low speed controller 512 is coupled to storage device 506 and low speed bus 514. The low-speed expansion port, which can include various communication ports, e.g., USB, Bluetooth, Ethernet, wireless Ethernet can be coupled to one or more input/output devices, such as a keyboard, a pointing device, microphone/speaker pair, a scanner, or a networking device such as a switch or router, e.g., through a network adapter.

The computing device 500 can be implemented in a number of different forms, as shown in the figure. For example, it can be implemented as a standard server 520, or multiple times in a group of such servers. It can also be implemented as part of a rack server system 524. In addition, it can be implemented in a personal computer such as a laptop computer 522. Alternatively, components from computing device 500 can be combined with other components in a mobile device (not shown), such as device 550. Each of such devices can contain one or more of computing device 500, 550, and an entire system can be made up of multiple computing devices 500, 550 communicating with each other.

The computing device 500 can be implemented in a number of different forms, as shown in the figure. For example, it can be implemented as a standard server 520, or multiple times in a group of such servers. It can also be implemented as part of a rack server system 524. In addition, it can be implemented in a personal computer such as a laptop computer 522. Alternatively, components from computing device 500 can be combined with other components in a mobile device (not shown), such as device 550. Each of such devices can contain one or more of computing device 500, 550, and an entire system can be made up of multiple computing devices 500, 550 communicating with each other.

Computing device 550 includes a processor 552, memory 564, and an input/output device such as a display 554, a communication interface 566, and a transceiver 568, among other components. The device 550 can also be provided with a storage device, such as a micro-drive or other device, to provide additional storage. Each of the components 550, 552, 564, 554, 566, and 568, are interconnected using various buses, and several of the components can be mounted on a common motherboard or in other manners as appropriate.

The processor 552 can execute instructions within the computing device 550, including instructions stored in the memory 564. The processor can be implemented as a chipset of chips that include separate and multiple analog and digital processors. Additionally, the processor can be implemented using any of a number of architectures. For example, the processor can be a CISC (Complex Instruction Set Computers) processor, a RISC (Reduced Instruction Set Computer) processor, or a MISC (Minimal Instruction Set Computer) processor. The processor can provide, for example, for coordination of the other components of the device 550, such as control of user interfaces, applications run by device 550, and wireless communication by device 550.

Processor 552 can communicate with a user through control interface 558 and display interface 556 coupled to a display 554. The display 554 can be, for example, a TFT (Thin-Film-Transistor Liquid Crystal Display) display or an OLED (Organic Light Emitting Diode) display, or other appropriate display technology. The display interface 556 can comprise appropriate circuitry for driving the display 554 to present graphical and other information to a user. The control interface 558 can receive commands from a user and convert them for submission to the processor 552. In addition, an external interface 562 can be provide in communication with processor 552, so as to enable near area communication of device 550 with other devices. External interface 562 can provide, for example, for wired communication in some implementations, or for wireless communication in other implementations, and multiple interfaces can also be used.

The memory 564 stores information within the computing device 550. The memory 564 can be implemented as one or more of a computer-readable medium or media, a volatile memory unit or units, or a non-volatile memory unit or units. Expansion memory 574 can also be provided and connected to device 550 through expansion interface 572, which can include, for example, a SIMM (Single In Line Memory Module) card interface. The expansion memory 574 can provide extra storage space for device 550, or can also store applications or other information for device 550. Specifically, expansion memory 574 can include instructions to carry out or supplement the processes described above, and can include secure information also. Thus, for example, expansion memory 574 can be provide as a security module for device 550, and can be programmed with instructions that permit secure use of device 550. In addition, secure applications can be provided via the SIMM cards, along with additional information, such as placing identifying information on the SIMM card in a non-hackable manner.

The memory can include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 564, expansion memory 574, or memory on processor 552 that can be received, for example, over transceiver 568 or external interface 562.

Device 550 can communicate wirelessly through communication interface 566, which can include digital signal processing circuitry where necessary. Communication interface 566 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through (radio-frequency) transceiver 568. In addition, short-range communication can occur, such as using a Bluetooth, Wi-Fi, or other such transceiver (not shown). In addition, GPS (Global Positioning System) receiver module 570 can provide additional navigation- and location-related wireless data to device 550, which can be used as appropriate by applications running on device 550.

Device 550 can also communicate audibly using audio codec 560, which can receive spoken information from a user and convert it to usable digital information. Audio codec 560 can likewise generate audible sound for a user, such as through a speaker, e.g., in a handset of device 550. Such sound can include sound from voice telephone calls, can include recorded sound, e.g., voice messages, music files, etc. and can also include sound generated by applications operating on device 550.

The computing device 550 can be implemented in a number of different forms, as shown in the figure. For example, it can be implemented as a cellular telephone 780. It can also be implemented as part of a smartphone 782, personal digital assistant, or other similar mobile device.

Various implementations of the systems and methods described here can be realized in digital electronic circuitry, integrated circuitry, specially designed ASICs (application specific integrated circuits), computer hardware, firmware, software, and/or combinations of such implementations. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, e.g., magnetic discs, optical disks, memory, Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device, e.g., a CRT (cathode ray tube) or LCD (liquid crystal display) monitor for displaying information to the user and a keyboard and a pointing device, e.g., a mouse or a trackball by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The systems and techniques described here can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the systems and techniques described here, or any combination of such back end, middleware, or front end components. The components of the system can be interconnected by any form or medium of digital data communication, e.g., a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

### EXAMPLES

The invention is further described in the following examples, which do not limit the scope of the invention described in the claims.

### Example 1: Physiologically-Based Pharmacokinetic Model to Predict the Pharmacokinetics of Nasally Administered Sumatriptan from Four Devices

The purpose of this Example was to develop a PBPK model to facilitate the prediction of the systemic pharmacokinetics of sumatriptan from in vitro nasal cast deposition data. In this Example, predictions of systemic PK were made from deposition data of a number of different devices, formulation viscosities and orientations to cover a wide but relevant range of deposition profiles. This allowed the impact of device differences and subject behaviour on systemic pharmacokinetics to be assessed.

The objectives of this Example were to:
1. Develop a PBPK model that takes in vitro nasal cast deposition data as input and outputs plasma concentration vs. time profiles.
2. Parameterise the model using in vitro, in vivo and clinical data from literature.
3. Compare the model predictions to clinical pharmacokinetic data from literature.
4. Predict the pharmacokinetics.
5. Conduct statistical analysis to assess the influence of device, angle, insertion depth, dose and viscosity on the pharmacokinetic summary parameters and any interactions between these factors.

The nasal deposition profiles for the following nasal delivery devices were utilized in this Example: Adella N2B (D03/L06), CPS, Preloaded Nozzle, UDS1 Adella N2B, UDS1 std, UDSp Liquide, VP7+232 N2B, VP7+CB18. The profiles for each of these devices are shown in the table below. In Table 3, in the formulation column, the units cP are centipoise, which is a measure of dynamic viscosity. In the orientation column, the first value represents the vertical angle, the second value is the horizontal angle, and the final value is how deep into the nostril of the nasal cast the device was inserted. The angles and depth were fixed using a guide that the nasal spray device slots into which fixes its orientation relative to the nasal cast.

TABLE 3
Device Profiles

| | | Dose | | |
|---|---|---|---|---|
| ID | Device | (uL) | Formulation | Orientation |
| 1 | Adella N2B (D03 / L06) | 50 | 37 cP | 40°/4º/15 mm |
| 2 | Adella N2B (D03 / L06) | 50 | 37 cP | 50°/4º/15 mm |
| 3 | CPS | 100 | 2cP | 30°/0°/10 mm |
| 4 | Preloaded Nozzle | 50 | 2cP | 40°/5º/15 mm |
| 5 | Preloaded Nozzle | 50 | 2cP | 50°/5°/15 mm |
| 6 | UDS1 Adella N2B | 100 | 2cP | 35°/5°/15 mm |
| 7 | UDS1 Adella N2B | 100 | 2cP | 45°/5°/15 mm |
| 8 | UDS1 Adella N2B | 100 | 2cP | 55°/5°/15 mm |
| 9 | UDS1 std | 100 | 2cP | 35°/5°/15 mm |
| 10 | UDS1 std | 100 | 2cP | 45°/5°/15 mm |
| 11 | UDS1 std | 100 | 2cP | 55°/5°/15 mm |
| 12 | UDSp Liquide | 50 | 2cP | 30°/5°/15 mm |
| 13 | UDSp Liquide | 50 | 2cP | 45°/5°/15 mm |
| 14 | VP7 + 232 N2B | 50 | 2cP | 45°/5°/15 mm |
| 15 | VP7 + CB18 | 100 | 2cP | 35°/5°/15 mm |
| 16 | VP7 + CB18 | 100 | 2cP | 45°/5°/15 mm |
| 17 | VP7 + CB18 | 100 | 2cP | 55°/5°/15 mm |

### PBPK Model

A multi-compartment PBPK model as described herein was used assuming well-stirred tissue and blood compartments (i.e. uniform distribution with each compartment) representing arterial blood, lung, adipose, bone, brain, heart, kidney, nose, muscle, skin, gut, spleen, liver, rest of body compartments, and venous blood. Clearance was assumed to occur in the liver and blood compartments. The nose was modelled by five compartments representing five different sections of the nasal cast.

Based on the model structure reported by M. S. Bogdanffy, S. R. D. R. Plowchalk, A. Jarabek and M. E. Andersen, “A biologically-based risk assessment for vinyl acetate-induced cancer and non-cancer inhalation toxicity,” Toxicological Sciences, vol. 51, pp. 19-35, 1999, the nasal compartments were separated into three sub-compartments representing the nasal mucus, epithelium and sub-epithelium. Mass transfer between the mucus, epithelium and sub-epithelium were modelled as permeation limited and between the sub-epithelium and the blood compartments as perfusion limited following Bogdanffy et al., Sumatriptan was modelled as if delivered via an aqueous solution therefore it was assumed that the deposited droplets distribute homogenously across the mucus at the point of deposition and the dosed volume is not significantly affect the mucus volume. A schematic of the systemic part of the model is shown in FIG. 1. The nose was further compartmentalized. A schematic of the nasal part of the model is shown in FIG. 2.

The blood flow to the nasal floor, turbinates, olfactory area and rhinopharynx was assumed to make up the entirety of the blood flow to the nose. Blood flow to the nasal vestibule and atrium (composing the nose area in FIG. 2) is known to be negligibly small so was set to 0 for this region (A. Pires, A. Fortuna, G. Alves and A. Falcão, “Intranasal drug delivery: How, why and what for?,” Journal of Pharmacy and Pharmaceutical Sciences, vol. 12, pp. 288-311, 2009).

Mucociliary clearance was modelled as a first order mass transport process pushing mass from the floor, turbinates and olfactory area into the rhinopharynx at a fixed anterior mucociliary clearance rate (I. Gonda and E. Gipps, “Model of Disposition of Drugs Administered into the Human Nasal Cavity,” Pharm Res, vol. 7, pp. 69-75, 1990). Note, the olfactory region is known to contain little to no motile cilia (S. Gänger and K. Schindowski, “Tailoring Formulations for Intranasal Nose-to-Brain Delivery: A Review on Architecture, Physico-Chemical Characteristics and Mucociliary Clearance of the Nasal Olfactory Mucosa,” Pharmaceutics, vol. 10, p. 116, 2018) however mass transport via gravitational or centrifugal sliding and the continuous production of mucus from Bowman's glands are thought to contribute to mass transport away from this region (T. T. Solbu and T. Holen, “Aquaporin Pathways and Mucin Secretion of Bowman's Glands Might Protect the Olfactory Mucosa,” Chemical Senses, vol. 37, pp. 35-46, 2012). Therefore, the anterior mucociliary clearance rate was also applied to the olfactory region, however in this case it represents different mechanisms of mass transport.

Transport from the rhinopharynx to the gastrointestinal tract was represented by the posterior transfer coefficient reported by Gonda and Gipps (I. Gonda and E. Gipps, “Model of Disposition of Drugs Administered into the Human Nasal Cavity,” Pharm Res, vol. 7, pp. 69-75, 1990). Due to the low oral bioavailability of sumatriptan (L. F. Lacey, H. E. K. and P. A. Fowler, “Single dose pharmacokinetics of sumatriptan in healthy volunteers,” European Journal of Clinical Pharmacology, vol. 47, pp. 543-548, 1995) and the low proportion of the dose that is likely to enter the gastrointestinal tract, the oral bioavailability was set to 0%. The physiological and compound specific parameters for the PBPK model used in this Example are outlined in the following table.

TABLE 4
Sources and Values of Initial Model Parameters.

| | Parameter | | |
|---|---|---|---|
| Parameter | Type | Value | Source |
| Q_{co} - Cardiac | Physiological | 5200.0 mL/min | R. P. Brown, M. D. Delp, |
| output | | | S. L. Lindstedt, L. R. |
| | | | Rhomberg and R. P. |
| | | | Beliles, “Physiological |
| | | | parameter values for |
| | | | physiologically based |
| | | | pharmacokinetic models.,” |
| | | | Toxicology and Industrial |
| | | | Health, vol. 13, pp. 407- |
| | | | 484, 1997 and references |
| | | | within |
| Qₙₒₛₑ - Blood | Physiological | 0.003Q_{co} | Notes for Table 1 in M. S. |
| flow to nose | | | Bogdanffy, S. R. D. R. |
| | | | Plowchalk, A. Jarabek and |
| | | | M. E. Andersen, “A |
| | | | biologically-based risk |
| | | | assessment for vinyl |
| | | | acetate-induced cancer and |
| | | | non-cancer inhalation |
| | | | toxicity,” Toxicological |
| | | | Sciences, vol. 51, pp. 19- |
| | | | 35, 1999 and references |
| | | | within |
| Q_{adipose} - Blood | Physiological | 0.052Q_{co} | Table 27 in R. P. Brown, |
| flow to adipose | | | M. D. Delp, S. L. |
| | | | Lindstedt, L. R. Rhomberg |
| | | | and R. P. Beliles, |
| | | | “Physiological parameter |
| | | | values for physiologically |
| | | | based pharmacokinetic |
| | | | models.,” Toxicology and |
| | | | Industrial Health, vol. 13, |
| | | | pp. 407-484, 1997and |
| | | | references within |
| Q_{bone} - Blood | Physiological | 0.042Q_{co} | Same as Q_{adipose} |
| flow to bone | | | |
| Q_{brain} - Blood | Physiological | 0.114Q_{co} | Same as Q_{adipose} |
| flow to brain | | | |
| Qₕₑₐᵣₜ - Blood | Physiological | 0.4Q_{co} | Same as Q_{adipose} |
| flow to heart | | | |
| Q_{kidney} - Blood | Physiological | 0.175Q_{co} | Same as Q_{adipose} |
| flow to kidneys | | | |
| Q_{muscle} - Blood | Physiological | 0.191Q_{co} | Same as Q_{adipose} |
| flow to muscle | | | |
| Qₛₖᵢₙ - Blood | Physiological | 0.058Q_{co} | Same as Q_{adipose} |
| flow to skin | | | |
| Q_{gut} - Blood | Physiological | 0.17Q_{co} | Table 5 in O. Luttringer, |
| flow to gut | | | F.-P. Thiel, P. Poulin, A. |
| | | | H. Schmitt-Hoffmann, T. |
| | | | W. Guentert and T. Lavé, |
| | | | “Physiologically Based |
| | | | Pharmacokinetic (PBPK) |
| | | | Modeling of Disposition of |
| | | | Epiroprim in Humans,” |
| | | | Journal of Pharmaceutical |
| | | | Sciences, vol. 92, pp. 1990- |
| | | | 2007, 2003 and references |
| | | | within |
| Qₛₚₗₑₑₙ - Blood | Physiological | 0.02Q_{co} | Same as Q_{gut} |
| flow to spleen | | | |
| Qₗᵢᵥₑᵣ - Blood | Physiological | 0.227Q_{co} | Same as Q_{adipose} |
| flow to liver | | | |
| Qᵣₑₛₜ - Blood flow to the rest of the body | Physiological | ${Q}_{CO} - {\sum }_{i \neq lung} {Q}_{i}$ | — |
| W - Body | Physiological | 70 kg | International Commission |
| weight | | | on Radiological Protection, |
| | | | “Human Respiratory Tract |
| | | | Model for Radiological |
| | | | Protection,” 1994 |
| ρ_{T} - Tissue | Physiological | 1.00 g mL⁻¹ | Table 19 and associated |
| density (apart | | | discussion in R. P. Brown, |
| from bone) | | | M. D. Delp, S. L. |
| | | | Lindstedt, L. R. Rhomberg |
| | | | and R. P. Beliles, |
| | | | “Physiological parameter |
| | | | values for physiologically |
| | | | based pharmacokinetic |
| | | | models.,” Toxicology and |
| | | | Industrial Health, vol. 13, |
| | | | pp. 407-484, 1997 |
| ρ_{bone} | Physiological | 1.99 g mL⁻¹ | R. P. Brown, M. D. Delp, |
| | | | S. L. Lindstedt, L. R. |
| | | | Rhomberg and R. P. |
| | | | Beliles, “Physiological |
| | | | parameter values for |
| | | | physiologically based |
| | | | pharmacokinetic models.,” |
| | | | Toxicology and Industrial |
| | | | Health, vol. 13, pp. 407- |
| | | | 484, 1997 and references |
| | | | within |
| V_{lung} - Volume | Physiological | 0.0076W/ρ_{T} | Table 7 in R. P. Brown, M. |
| of lung tissue | | | D. Delp, S. L. Lindstedt, L. |
| | | | R. Rhomberg and R. P. |
| | | | Beliles, “Physiological |
| | | | parameter values for |
| | | | physiologically based |
| | | | pharmacokinetic models.,” |
| | | | Toxicology and Industrial |
| | | | Health, vol. 13, pp. 407- |
| | | | 484, 1997and references |
| | | | within |
| V_{adipose} - | Physiological | 0.2142W/ρ_{T} | Same as V_{lung} |
| Volume of | | | |
| adipose | | | |
| V_{bone} - Volume | Physiological | 0.1429W/ρ_{bone} | Same as V_{lung} |
| of bone | | | |
| V_{brain} - Volume | Physiological | 0.02W/ρ_{T} | Same as V_{lung} |
| of brain tissue | | | |
| Vₕₑₐᵣₜ - Volume | Physiological | 0.0047W/ρ_{T} | Same as V_{lung} |
| of heart tissue | | | |
| V_{kidney} - | Physiological | 0.0044W/ρ_{T} | Same as V_{lung} |
| Volume of | | | |
| kidney tissue | | | |
| V_{muscle} - | Physiological | 0.4W/ρ_{T} | Same as V_{lung} |
| Volume of | | | |
| muscle tissue | | | |
| Vₛₖᵢₙ - Volume | Physiological | 0.0371W/ρ_{T} | Same as V_{lung} |
| of skin tissue | | | |
| V_{gut} - Volume | Physiological | 0.0171W/ρ_{T} | Same as V_{lung} |
| of gut tissue | | | |
| Vₛₚₗₑₑₙ - | Physiological | 0.0026W/ρ_{T} | Same as V_{lung} |
| Volume of | | | |
| spleen tissue | | | |
| Vₗᵢᵥₑᵣ - Volume | Physiological | 0.0257W/ρ_{T} | Same as V_{lung} |
| of liver tissue | | | |
| Vₐᵣₜ - Volume | Physiological | 0.05925W/ρ_{T} | Table 21 and associated |
| of arterial blood | | | discussion on page 54 of R. |
| | | | P. Brown, M. D. Delp, S. |
| | | | L. Lindstedt, L. R. |
| | | | Rhomberg and R. P. |
| | | | Beliles, “Physiological |
| | | | parameter values for |
| | | | physiologically based |
| | | | pharmacokinetic models.,” |
| | | | Toxicology and Industrial |
| | | | Health, vol. 13, pp. 407- |
| | | | 484, 1997 |
| Vᵥₑₙ - Volume | Physiological | 0.01975W/ρ_{T} | Same as Vₐᵣₜ |
| of venous blood | | | |
| Vᵣₑₛₜ - Volume of tissue in the rest of the body | Physiological | $( W - {ρ}_{T} {\sum }_{i \neq bone} ( {V}_{i} ) - {ρ}_{bone} {V}_{bone} ) / {ρ}_{T}$ | — |
| K_{pu,lung} - | Compound | 2.53 | Estimated using the |
| Tissue: plasma | | | method of Rodgers and |
| partition | | | Rowland (T. Rodgers, D. |
| coefficient for | | | Leahy and M. Rowland, |
| the lung | | | “Physiologically based |
| K_{pu,adipose} - | Compound | 9.83 | pharmacokinetic modeling |
| Tissue: plasma | | | 1: Predicting the tissue |
| partition | | | distribution of moderate-to- |
| coefficient for | | | strong bases,” Journal of |
| the adipose | | | Pharmaceutical Sciences, |
| K_{pu,bone} - | Compound | 1.44 | vol. 94, pp. 1259-1276, |
| Tissue: plasma | | | 2005) using the LogP and |
| partition | | | fraction unbound in plasma |
| coefficient for | | | detailed in this table |
| the bone | | | |
| K_{pu,brain} - | Compound | 29.88 | |
| Tissue: plasma | | | |
| partition | | | |
| coefficient for | | | |
| the brain | | | |
| K_{pu,heart} - | Compound | 4.98 | |
| Tissue: plasma | | | |
| partition | | | |
| coefficient for | | | |
| the heart | | | |
| K_{pu,kidney} - | Compound | 6.10 | |
| Tissue: plasma | | | |
| partition | | | |
| coefficient for | | | |
| the kidney | | | |
| K_{pu,muscle} - | Compound | 3.23 | |
| Tissue: plasma | | | |
| partition | | | |
| coefficient for | | | |
| the muscle | | | |
| K_{pu,skin} - | Compound | 20.51 | |
| Tissue: plasma | | | |
| partition | | | |
| coefficient for | | | |
| the skin | | | |
| K_{pu,gut} - | Compound | 5.56 | |
| Tissue: plasma | | | |
| partition | | | |
| coefficient for | | | |
| the gut | | | |
| K_{pu,spleen} - | Compound | 3.27 | |
| Tissue: plasma | | | |
| partition | | | |
| coefficient for | | | |
| the spleen | | | |
| K_{pu,liver} - | Compound | 4.21 | |
| Tissue: plasma | | | |
| partition | | | |
| coefficient for | | | |
| the liver | | | |
| K_{pu,nose} - | Compound | Assumed to be | |
| Tissue: plasma | | equal to that of | |
| partition | | the lung | |
| coefficient for | | | |
| the nose | | | |
| K_{pu,rest} - | Compound | Mean of the | |
| Tissue: plasma | | non-adipose | |
| partition | | tissue:plasma | |
| coefficient for | | partition | |
| the rest of the | | coefficients | |
| body | | | |
| f_{K}_{ₚᵤ} | Compound | 1 | Parameter adjustment to |
| | | | achieve agreement with |
| | | | test product PK data |
| R - | Compound | 1 | B. L. Morse, A. Kolur, L. |
| Blood/plasma | | | R. Hudson, A. T. Hogan, |
| ratio | | | L. H. Chen, R. M. |
| | | | Brackman, G. A. Sawada, |
| | | | J. K. Fallon, P. C. Smith |
| | | | and K. M. Hillgren, |
| | | | “Pharmacokinetics of |
| | | | organic cation transporter 1 |
| | | | (OCT1) substrates in |
| | | | Oct1/2 knockout mice and |
| | | | species difference in |
| | | | hepatic OCtl-mediated |
| | | | uptake S,” Drug |
| | | | Metabolism and |
| | | | Disposition, vol. 48, pp. |
| | | | 93-105, 2020 |
| fᵤ - fraction of | Compound | 0.63 | J. C. Kalvass, T. S. Maurer |
| sumatriptan | | | and G. M. Pollack, “Use of |
| unbound in | | | plasma and brain unbound |
| blood plasma | | | fractions to assess the |
| | | | extent of brain distribution |
| | | | of 34 drugs: Comparison of |
| | | | unbound concentration |
| | | | ratios to in vivo P- |
| | | | glycoprotein efflux ratios,” |
| | | | Drug Metabolism and |
| | | | Disposition, vol. 35, pp. |
| | | | 660-666, 2007 |
| LogP | Compound | 0.93 | https://pubchem.ncbi.nlm.n |
| | | | ih.gov/compound/Sumatriptan |
| pKa | Compound | 4.9, 9,5 | https://pubchem.ncbi.nlm.n |
| | | | ih.gov/compound/Sumatripta |
| k_{mcc,ant} - | Physiological | 0.0625 hr⁻¹ | I. Gonda and E. Gipps, |
| Anterior | | | “Model of Disposition of |
| mucociliary | | | Drugs Administered into |
| clearance rate | | | the Human Nasal Cavity,” |
| | | | Pharm Res, vol. 7, pp. 69- |
| | | | 75, 1990 |
| k_{mcc,post} - | Physiological | 4.167 hr⁻¹ | Same as k_{mcc,ant} |
| Posterior | | | |
| mucociliary | | | |
| clearance rate | | | |
| Aᵥₐₗᵥₑ - Area of | Physiological | 40128 mm² | Internal Pharma data |
| the nasal valve | | | |
| A_{floor} - Area of | Physiological | 4019 mm² | Internal Pharma data |
| the nasal floor | | | |
| A_{turb} - Area of | Physiological | 9057 mm² | Internal Pharma data |
| the turbinates | | | |
| A_{olf} - Area of | Physiological | 9743 mm² | Internal Pharma data |
| the olfactory | | | |
| region | | | |
| Aᵣₕᵢₙₒ - Area of | Physiological | 2919 mm² | Internal Pharma data |
| the | | | |
| rhinopharynx | | | |
| Qᵥₐₗᵥₑ - Blood | Physiological | 0.1Qₙₒₛₑ | Estimated |
| flow to the | | | |
| nasal valve | | | |
| Q_{floor} - Blood flow to the nasal floor | Physiological | $0.45 \frac{{Q}_{nose} {A}_{floor}}{{A}_{floor} + {A}_{turb} + {A}_{rhino}}$ | Following method outlined in M. S. Bogdanffy, S. R. D. R. Plowchalk, A. Jarabek and M. E. |
| | | | Andersen, “A biologically- |
| | | | based risk assessment for |
| | | | vinyl acetate-induced |
| | | | cancer and non-cancer |
| | | | inhalation toxicity,” |
| | | | Toxicological Sciences, |
| | | | vol. 51, pp. 19-35, 1999 |
| Q_{turb} - Blood flow to the turbinates | Physiological | $0.45 \frac{{Q}_{nose} {A}_{turb}}{{A}_{floor} + {A}_{turb} + {A}_{rhino}}$ | Same as Q_{floor} |
| Q_{olf} - Blood | Physiological | 0.45Qₙₒₛₑ | Same as Q_{floor} |
| flow to the | | | |
| olfactory region | | | |
| Qᵣₕᵢₙₒ - Blood flow to the rhinopharynx | Physiological | $0.45 \frac{{Q}_{nose} {A}_{rhino}}{{A}_{floor} + {A}_{turb} + {A}_{rhino}}$ | Same as Q_{floor} |
| T_{valve,muc} - | Physiological | 10 μm | C. B. Frederick, M. L. |
| Thickness of | | | Bush, L. G. Lomax, K. A. |
| mucus in the | | | Black, L. Finch, J. S. |
| nasal valve | | | Kimbell, K. T. Morgan, R. |
| | | | P. Subramaniam, J. B. |
| | | | Morris and J. S. Ultman, |
| | | | “Application of a hybrid |
| | | | computational fluid |
| | | | dynamics and |
| | | | physiologically based |
| | | | inhalation model for |
| | | | interspecies dosimetry |
| | | | extrapolation of acidic |
| | | | vapors in the upper |
| | | | airways,” Toxicology and |
| | | | Applied Pharmacology, |
| | | | vol. 152, pp. 211-231, 1998 |
| T_{floor,muc} - | Physiological | 10 μm | Same as T_{valve,muc} |
| Thickness of | | | |
| mucus in the | | | |
| nasal floor | | | |
| T_{turb,muc} - | Physiological | 10 μm | Same as T_{valve,muc} |
| Thickness of | | | |
| mucus in the | | | |
| turbinates | | | |
| T_{olf,muc} - | Physiological | 20 μm | Same as T_{valve,muc} |
| Thickness of | | | |
| mucus in the | | | |
| olfactory region | | | |
| T_{rhino,muc} - | Physiological | 10 μm | Same as T_{valve,muc} |
| Thickness of | | | |
| mucus in the | | | |
| rhinopharynx | | | |
| T_{valve,ep} - | Physiological | 25 μm | Same as T_{valve,muc} |
| Thickness of | | | |
| epithelium in | | | |
| the nasal valve | | | |
| T_{floor,ep} - | Physiological | 25 μm | Same as T_{valve,muc} |
| Thickness of | | | |
| mucus in the | | | |
| nasal floor | | | |
| T_{turb,ep} - | Physiological | 25 μm | Same as T_{valve,muc} |
| Thickness of | | | |
| mucus in the | | | |
| turbinates | | | |
| T_{olf,ep} - | Physiological | 80 μm | Same as T_{valve,muc} |
| Thickness of | | | |
| epithelium in | | | |
| the olfactory | | | |
| region | | | |
| T_{rhino,ep} - | Physiological | 25 μm | Same as T_{valve,muc} |
| Thickness of | | | |
| epithelium in | | | |
| the | | | |
| rhinopharynx | | | |
| T_{valve,sub} - | Physiological | 75 μm | Same as T_{valve,muc} |
| Thickness of | | | |
| subepithlium in | | | |
| the nasal valve | | | |
| T_{floor,sub} - | Physiological | 75 μm | Same as T_{valve,muc} |
| Thickness of | | | |
| subepithelium in | | | |
| the nasal floor | | | |
| T_{turb,sub} - | Physiological | 75 μm | Same as T_{valve,muc} |
| Thickness of | | | |
| mucus in the | | | |
| turbinates | | | |
| T_{olf,sub} - | Physiological | 50 μm | Same as T_{valve,muc} |
| Thickness of | | | |
| subepithelium in | | | |
| the olfactory | | | |
| region | | | |
| T_{rhino,sub} - | Physiological | 75 μm | Same as T_{valve,muc} |
| Thickness of | | | |
| subepithelium in | | | |
| the | | | |
| rhinopharynx | | | |
| Clₕ - Hepatic | Compound | — | Unknown |
| clearance of | | | |
| sumatriptan | | | |
| Cl_{blood} - Blood | Compound | — | Unknown |
| clearance of | | | |
| sumatriptan | | | |
| P - | Compound | 107 × 10⁻⁷ cm/s | Table 1 of N. Sibinovska, |
| Permeability in | | 62.7 × 10⁻⁷ cm/s | S. Žakelj, J. Trontelj and |
| nasal epithelium | | 1.39 × 10⁻⁷ cm/s | K. Kristan, “Applicability |
| | | | of RPMI 2650 and Calu-3 |
| | | | Cell Models for Evaluation |
| | | | of Nasal Formulations,” |
| | | | Pharmaceutics, vol. 14, pp. |
| | | | 1-19, 2021 |

The final model parameters are presented in Table 5. Any parameters not included here are equal to their values in Table 4.

TABLE 5
Sources and Values of Final Model Parameters

| Parameter | Parameter Type | Value |
|---|---|---|
| Clₕ - Hepatic clearance of | Compound | 30,000 hr⁻¹ |
| sumatriptan | | |
| Cl_{blood} - Blood clearance | Compound | 1000 hr⁻¹ |
| of sumatriptan | | |
| P - Permeability in nasal | Compound | 12.54 × 10⁻⁷ cm/s |
| epithelium | | |
| f_{K}_{ₚᵤ} | Compound | 1.8 |

The mathematical equations describing the models were solved using Python version 3.10.4 and SciPy 1.8.1.

### PBPK Model Verification and Modification

The model predictions were compared to the clinical data reported by C. Duquesnoy, J. P. Mamet, D. Sumner and E. Fuseau, “Comparative clinical pharmacokinetics of single doses of sumatriptan following subcutaneous, oral, rectal and intranasal administration,” European Journal of Pharmaceutical Sciences, vol. 6, pp. 99-104, 1998. For this the inputs to the model were the in vitro nasal cast deposition data for the USD-L standard device at multiple orientations (see Table 6 below) and a dose of 20 mg. The nasal epithelial permeability, scaling of the tissue:plasma partition coefficients, hepatic clearance and clearance from the blood were all varied to improve the agreement between the model and clinical data. The model was also tested for agreement with the reported nasal bioavailability of 16%.

TABLE 6
In Vitro Nasal Cast Deposition Fractions for a 2 cP Formulation
Delivered from the USDI Standard Device at Different
Orientations

| | Nasal | | | Olfactory | Rhino- |
|---|---|---|---|---|---|
| Orientation | Valve | Floor | Turbinates | Area | pharynx |
| 45°/5°/15 | 0.25 | 0.01 | 0.72 | 0.02 | 0.01 |
| mm | | | | | |
| 45°/5°/15 | 0.21 | 0.3 | 0.46 | 0.03 | 0 |
| mm | | | | | |
| 45°/5°/15 | 0.23 | 0 | 0.74 | 0.02 | 0.01 |
| mm | | | | | |
| 45°/5°/15 | 0.32 | 0 | 0.64 | 0.02 | 0.01 |
| mm | | | | | |
| 45°/5°/15 | 0.24 | 0 | 0.73 | 0.02 | 0.01 |
| mm | | | | | |
| 45°/5°/15 | 0.25 | 0 | 0.72 | 0.02 | 0.01 |
| mm | | | | | |
| 35°/5°/15 | 0.17 | 0.23 | 0.53 | 0.06 | 0.01 |
| mm | | | | | |
| 55°/5°/15 | 0.57 | 0.01 | 0.39 | 0.04 | 0 |
| mm | | | | | |
| 55°/5°/15 | 0.53 | 0.02 | 0.43 | 0.02 | 0 |
| mm | | | | | |
| 55°/5°/15 | 0.49 | 0.19 | 0.29 | 0.02 | 0 |
| mm | | | | | |
| 35°/5°/15 | 0.13 | 0 | 0.8 | 0.06 | 0.01 |
| mm | | | | | |
| 35°/5°/15 | 0.11 | 0 | 0.83 | 0.05 | 0.01 |
| mm | | | | | |
| 35°/5°/15 | 0.09 | 0 | 0.85 | 0.04 | 0.01 |
| mm | | | | | |

### Model Application-Prediction of Systemic Pharmacokinetics

After the model was built and verified against clinical pharmacokinetic data, systemic pharmacokinetics data were predicted for each of the orientations outlined in Table 3. A total of 1000 time points over 0-6 hours were be simulated to capture the maximum plasma concentration and for the area under the curve (AUC). The plasma concentrations vs. time profiles were plotted and the Cₘₐₓ and the area under the curve to the final time point (AUC₀₋ₜ) were reported for each configuration.

### Model Application—Statistical Analysis

The predicted pharmacokinetic outcomes were then plotted against each of the input factors using JMP 15.1.

### Results

### PBPK Model Verification and Modification

Initially, the model was used to predict the systemic pharmacokinetics of the input UDSI std data (see “Model Building” section in the Detailed Description) using the initial estimate parameters presented in Table 4. The initial predictions are presented in FIG. 7 alongside the clinical pharmacokinetics of nasally administered sumatriptan solution reported in C. Duquesnoy, J. P. Mamet, D. Sumner and E. Fuseau, “Comparative clinical pharmacokinetics of single doses of sumatriptan following subcutaneous, oral, rectal and intranasal administration,” European Journal of Pharmaceutical Sciences, vol. 6, pp. 99-104, 1998.

The initially predicted slow absorption suggested that the some parameter adjustment was necessary. The parameters with large amounts of uncertainty (or those that were simply unknown) were:
Nasal epithelial permeability (values of 1.39×10⁻⁷, 62.7×10⁻⁷ cm/s and 107×10⁻⁷ cm/s reported in N. Sibinovska, S. Žakelj, J. Trontelj and K. Kristan, “Applicability of RPMI 2650 and Calu-3 Cell Models for Evaluation of Nasal Formulations,” Pharmaceutics, vol. 14, pp. 1-19, 2021);
Tissue:plasma partition coefficient scale factor (unknown, commonly used to adjust line shape (S. A. Peters, “Evaluation of a generic physiologically based pharmacokinetic model for lineshape analysis,” Clinical Pharmacokinetics, vol. 47, pp. 261-275, 2008));
Hepatic clearance (unknown);
Clearance from the blood (unknown).

First the hepatic clearance was adjusted as this is known to saturate at larger values. The saturation value for this system was found to be 30,000 hr⁻¹ which still did not allow the drug to clear from systemic circulation as observed in the clinical data shown in FIG. 7 so the value was fixed at this saturation point.

The permeability was then scaled to increase the rate of release from the nasal epithelium into systemic circulation. A value of 12.54×10⁻⁷ cm/s was found to bring the curve close to the clinical data and lies between the values reported in N. Sibinovska, S. Žakelj, J. Trontelj and K. Kristan, “Applicability of RPMI 2650 and Calu-3 Cell Models for Evaluation of Nasal Formulations,” Pharmaceutics, vol. 14, pp. 1-19, 2021.

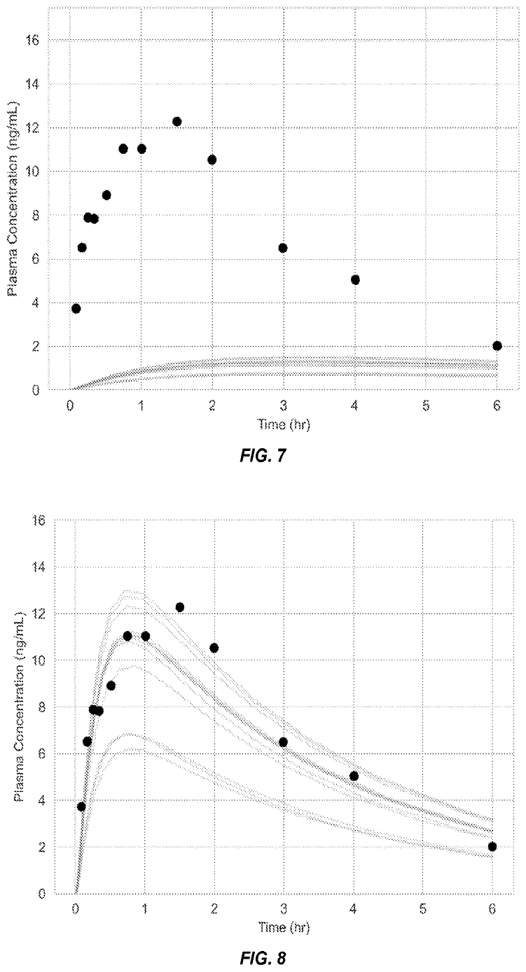

At this point, the tissue:plasma partition coefficient was increased from 1 to 1.8 to reduce the amount of drug available to partition from all tissue to the plasma. Finally, the clearance from the blood was increased from 0 to 1000 hr⁻¹ to further control the lineshape of systemic elimination and bring the predictions into visual agreement with the clinical pharmacokinetics. The final optimised model predictions are presented in FIG. 8.

The model predictions were then finally verified against the known nasal bioavailability of Imigran nasal spray of 16% (GlaxoSmithKline, “IMIGRAN® NASAL SPRAY,” [Online]. Available:

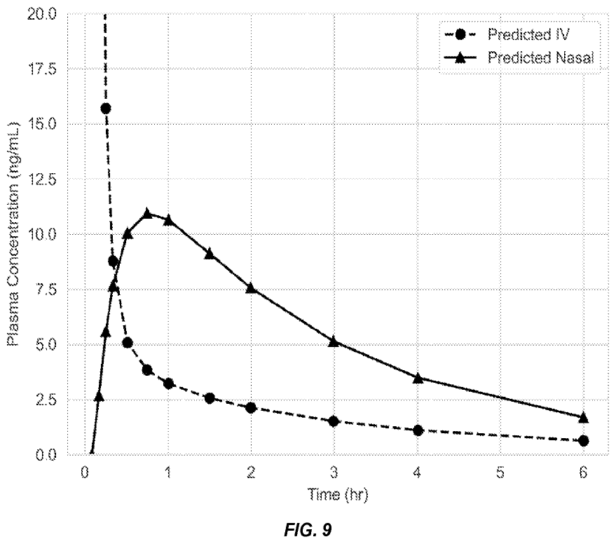

https://gskpro.com/content/dam/global/hcpportal/en_NA/PI/Imigran-20-mg-Nasal-Spray-GDS07.pdf) by simulating a 20 mg intravenous bolus dose and a nasal dose (using the nasal deposition pattern of the first row in Table 4) at the time points reported by C. Duquesnoy, J. P. Mamet, D. Sumner and E. Fuseau, “Comparative clinical pharmacokinetics of single doses of sumatriptan following subcutaneous, oral, rectal and intranasal administration,” European Journal of Pharmaceutical Sciences, vol. 6, pp. 99-104, 1998 and taking the ratios of the AUC₀₋ₜ values. The predicted nasal bioavailability of sumatriptan using the model was 21.59%, close to the reported clinical value, and the predicted plasma concentration vs. time profiles used to calculate this are presented in FIG. 9.

### Model Application-Prediction of Systemic Pharmacokinetics

Following the successful verification of the model, the systemic pharmacokinetics of each of the nasal cast deposition profiles presented in Table 3 were input into the model alongside a dose of 20 mg sumatriptan. The nasal cast deposition results for each of the configurations are presented in Table 7.

TABLE 7
Nasal Cast Deposition Data Input into the Model

| | Nose | | | | | Filter/ |
|---|---|---|---|---|---|---|
| | Area | Floor | Turbinates | Olfactory | Rhinopharynx | lungs |
| ID | (%) | (%) | (%) | Area (%) | (%) | (%) |
| 1 | 5% | 0% | 92% | 3% | 0% | 0% |
| 2 | 0% | 0% | 98% | 2% | 0% | 0% |
| 3 | 26% | 66% | 9% | 0% | 0% | 0% |
| 4 | 2% | 0% | 53% | 42% | 2% | 0% |
| 5 | 15% | 2% | 35% | 48% | 0% | 0% |
| 6 | 8% | 6% | 80% | 6% | 1% | 0% |
| 7 | 14% | 0% | 79% | 7% | 0% | 0% |
| 8 | 35% | 0% | 60% | 5% | 0% | 0% |
| 9 | 13% | 6% | 75% | 5% | 1% | 0% |
| 10 | 25% | 5% | 67% | 2% | 1% | 0% |
| 11 | 53% | 7% | 37% | 3% | 0% | 0% |
| 12 | 12% | 3% | 53% | 28% | 4% | 0% |
| 13 | 21% | 0% | 41% | 37% | 1% | 0% |
| 14 | 6% | 0% | 34% | 60% | 0% | 0% |
| 15 | 7% | 7% | 79% | 7% | 1% | 0% |
| 16 | 12% | 15% | 68% | 5% | 0% | 0% |
| 17 | 21% | 20% | 55% | 5% | 0% | 0% |

The predicted pharmacokinetic parameters are presented in Table 8.

TABLE 8
Predicted Pharmacokinetic Parameters from the Nasal Cast
Deposition Data Presented in Table 7

| ID | Cₘₐₓ (ng/ml) | AUC₀₋₁ (ng hr/mL) | Tₘₐₓ (min) |
|---|---|---|---|
| 1 | 11.81 | 41.31 | 0.69 |
| 2 | 12.45 | 37.32 | 0.62 |
| 3 | 8.08 | 36.48 | 0.61 |
| 4 | 8.43 | 49.88 | 0.83 |
| 5 | 6.56 | 47.53 | 0.79 |
| 6 | 10.90 | 44.25 | 0.74 |
| 7 | 10.22 | 46.09 | 0.77 |
| 8 | 7.82 | 41.36 | 0.69 |
| 9 | 10.34 | 45.07 | 0.75 |
| 10 | 9.05 | 35.43 | 0.59 |
| 11 | 5.49 | 35.62 | 0.59 |
| 12 | 8.14 | 44.82 | 0.75 |
| 13 | 6.70 | 47.67 | 0.79 |
| 14 | 6.71 | 54.58 | 0.91 |
| 15 | 10.99 | 44.51 | 0.74 |
| 16 | 10.32 | 45.32 | 0.76 |
| 17 | 9.19 | 37.60 | 0.63 |

### Discussion

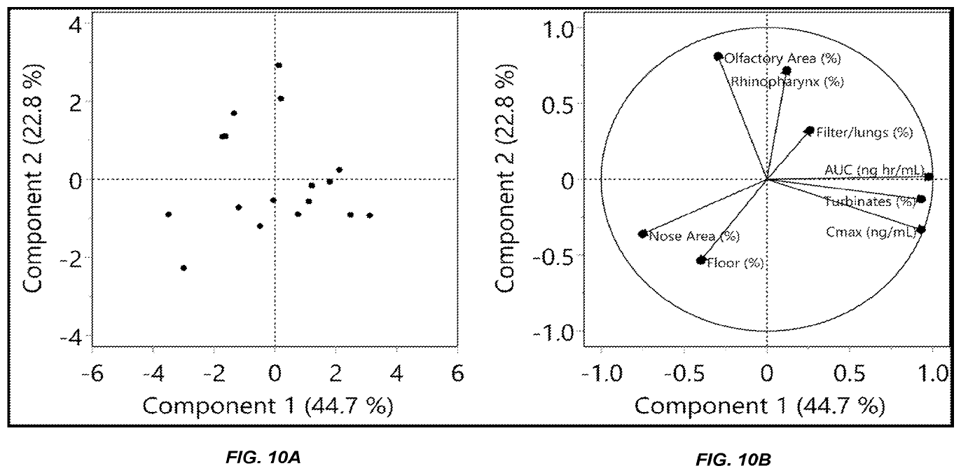

In the initial project plan, a linear model was to be used to assess the most impactful factors out of device and orientation, however in the updated scope a larger number of device parameters were screened so initially the relationships between the nasal cast deposition data and the predicted pharmacokinetics were examined to establish in vitro-in silico relationships. Principal component analysis (PCA) was first used to examine the relationships between nasal cast deposition fractions and predicted pharmacokinetic parameters. The PCA biplot is presented in FIGS. 10A-10B.

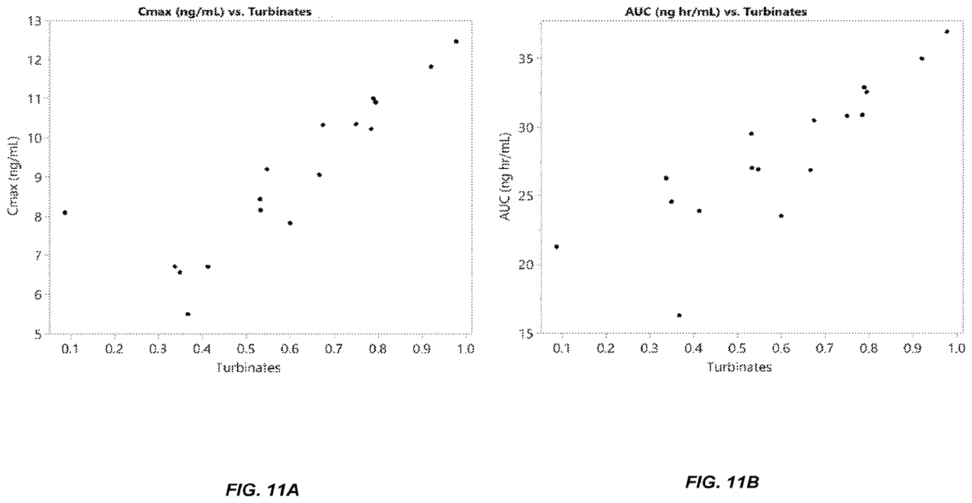

The PCA of the factor weighting vectors indicated correlations between parameters. Parallel vectors with the same direction indicate positive correlations. Parallel vectors with the opposite directions indicate negative correlations. Orthogonal vectors indicate no correlation. The vectors in FIG. 10B show strong correlations between turbinate deposition and predicted AUC and Cₘₐₓ and limited correlations with the other individual nasal regions. To investigate this further, plots of each of the nasal region deposition fractions and the Cₘₐₓ and AUC₀₋ₜ were examined. The relationships between the turbinate deposition and the Cₘₐₓ and AUC₀₋ₜ are presented in FIGS. 11A-11B.

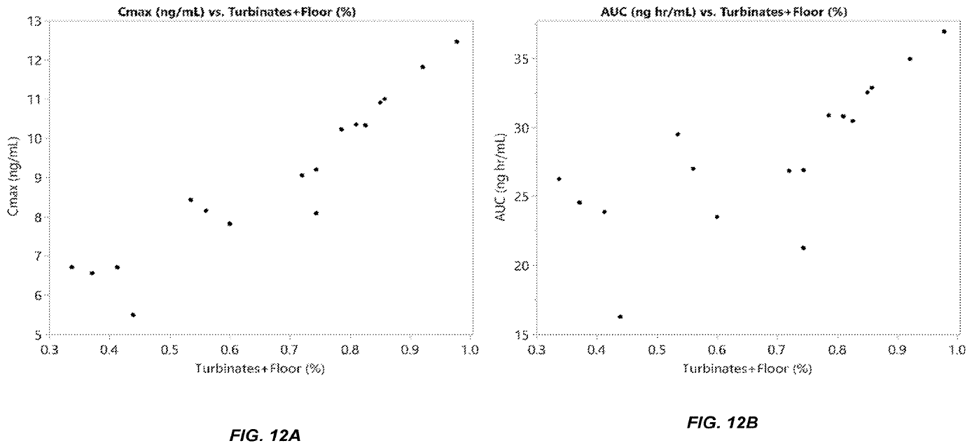

These data show a strong positive correlation between deposition in the turbinates and predicted systemic exposure. The correlation was stronger for Cₘₐₓ then for AUC₀₋ₜ, however there is a single outlying point on the Cₘₐₓ plot at low turbinate deposition. On inspection this was found to be the CPS pump (ID 3 in Table 3, Table 7Table, Table 8) which showed higher deposition in the floor region (66% compared to a maximum of 20% for all other data) than the other devices. This indicates that combined deposition in the nasal floor and turbinates is important for systemic exposure. This combination was plotted against the predicted systemic exposure, shown in FIGS. 12A-12B.

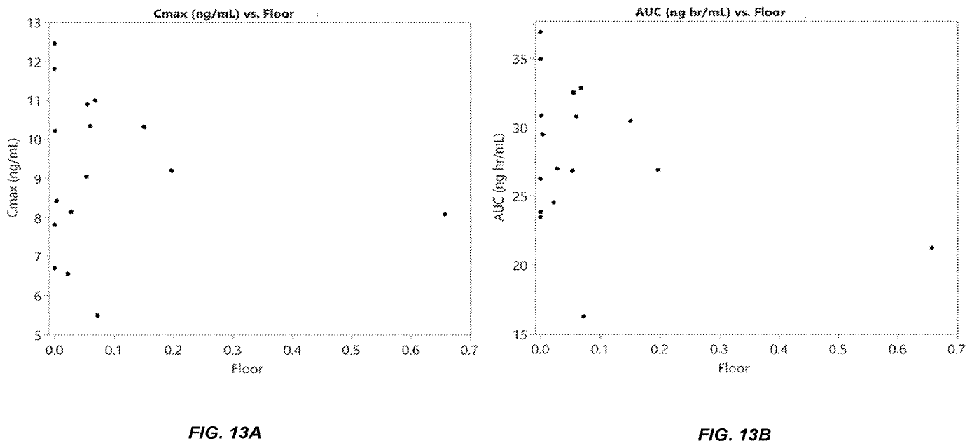

Comparison between fractional deposition in the turbinates and the floor correlates more strongly with the predicted Cₘₐₓ but appears to increase the scatter of the relationship with AUC₀₋ₜ. This indicates that both parameters are important in driving the system. At this stage, a stronger conclusion for the nasal floor dependence cannot be made as a more limited range of nasal floor deposition values were input into the model as evidenced in FIGS. 13A-13B, which shows the relationship between fractional deposition on the nasal floor and predicted systemic exposure.

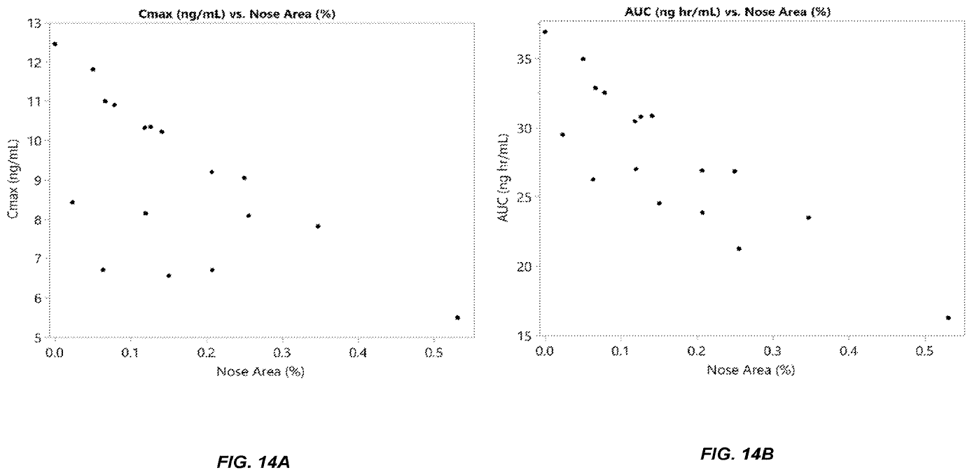

The PCA showed limited correlations between predicted systemic exposure and deposition in the olfactory region, rhinopharynx, and filter/lungs; therefore these were not plotted. The nose area was investigated as, despite showing limited correlation in PCA, the impact of a region that does not permit absorption to systemic circulation was of interest. The relationships between Cₘₐₓ (left) and AUC₀₋ₜ (right) and fractional deposition in the nose are presented in FIGS. 14A-14B.

These data demonstrate that there is a somewhat negative relationship between nose area deposition and systemic pharmacokinetics. This can be explained by the lack of absorption pathway in this region of the nose, however there are some points at low nose areas that show both low and high Cₘₐₓ and AUC₀₋ₜ suggesting that deposition in other regions also must be impacting predicted systemic PK.

The conclusions from this section are that the predicted systemic exposure is determined in a large part by the deposition in the turbinates. This follows from the region showing high surface area compared to the other regions apart from the olfactory region (9057 mm² compared to 4019 mm² in the nasal floor, 2919 mm² in the rhinopharynx and 9743 mm² in the olfactory region) and thin mucus (25 μm compared to 80 μm in the olfactory region (C. B. Frederick, M. L. Bush, L. G. Lomax, K. A. Black, L. Finch, J. S. Kimbell, K. T. Morgan, R. P. Subramaniam, J. B. Morris and J. S. Ultman, “Application of a hybrid computational fluid dynamics and physiologically based inhalation model for interspecies dosimetry extrapolation of acidic vapors in the upper airways,” Toxicology and Applied Pharmacology, vol. 152, pp. 211-231, 1998)) and epithelial (10 μm compared to 10 μm in the olfactory region (C. B. Frederick, M. L. Bush, L. G. Lomax, K. A. Black, L. Finch, J. S. Kimbell, K. T. Morgan, R. P. Subramaniam, J. B. Morris and J. S. Ultman, “Application of a hybrid computational fluid dynamics and physiologically based inhalation model for interspecies dosimetry extrapolation of acidic vapors in the upper airways,” Toxicology and Applied Pharmacology, vol. 152, pp. 211-231, 1998)) layers. Drug targeting the central nasal cavity therefore has a high probability of landing in the turbinates and does not have to permeate far through the mucus and epithelial layers before reaching the perfused subepithelial tissue where it can permeate into capillaries and be transported into systemic circulation. Drug landing in the olfactory region must permeate through a much larger space before it reaches the perfused tissue, therefore not contributing as strongly to systemic exposure.

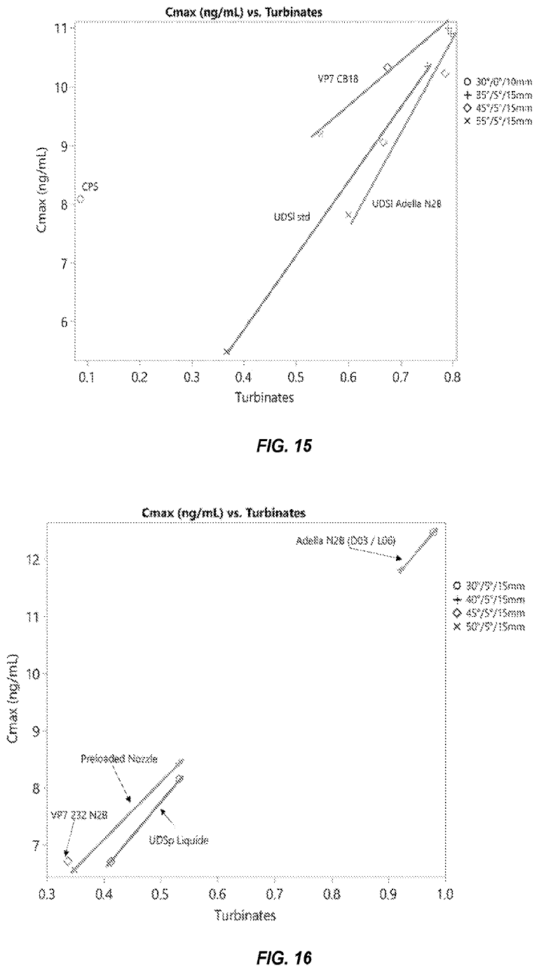

Using the insight developed in the previous section, differences in predicted systemic exposure for different device types and orientations were analysed. To ensure valuable comparisons, the 100 μL dosing volume devices (CPS, UDS1 Adella N2B (D03/L06), UDS1 std and VP7 CB18) were compared separately to the 50 μL devices (Adella N2B, Preloaded Nozzle, UDSp Liquide and VP7 232 N2B). The results for the 100 μL devices are presented in FIG. 15.

These data show that there is a general increase in predicted Cₘₐₓ with increased turbinate deposition (as discussed above) and that, for all devices apart from the CPS, there is an increase in both turbinate deposition and predicted Cₘₐₓ with decreasing insertion angle where 55° produced the lowest predicted Cₘₐₓ and 35° produced the highest predicted Cₘₐₓ. At high turbinate deposition the UDS1 Adella N2B, UDS1 std and VP7 CB18 all produce similar predicted Cₘₐₓ. The dependences between turbinate deposition and predicted Cₘₐₓ diverge at lower turbinate deposition. The VP7 CB18 shows higher predicted Cₘₐₓ at turbinate deposition fractions of 0.5-0.6 and a weaker dependence on increasing deposition. The reason for this appears to be increased deposition in the nasal floor for the VP7 CB18, which dampens the impact of variations in turbinate deposition. This suggests that variability due to differences in angle may be reduced for the VP7 CB18 compared to the other two devices. This relationship also explains the high predicted Cₘₐₓ of the CPS device despite its low turbinate deposition, as this device showed a much greater nasal floor deposition fraction than the other devices. The relationships between turbinate deposition fraction and predicted Cₘₐₓ for different 50 μL devices and orientations are presented in FIG. 16.

The relationships for the 50 μL devices are more straightforward than for the 100 μL devices as the 50 μL devices all show a similar dependence between turbinate deposition and predicted Cₘₐₓ. This is likely due to the nasal floor deposition for these devices all being low and not affecting the predicted systemic exposure. The turbinate deposition (the therefore the predicted Cₘₐₓ) for the 50 μL devices also increases with decreasing insertion angle. The differences in predicted Cₘₐₓ between the devices therefore depend on the ability of each device to deliver to the turbinates, with the Adella N2B (D03/L06) showing the highest and the other devices covering a similar range.

In conclusion, a PBPK model for the systemic absorption of sumatriptan from a nasally delivered solution was successfully developed and verified against clinical pharmacokinetic data.

### Example 2: Nose-to-Brain Physiologically Based Pharmacokinetic Model Building

The purpose of this study was to develop a PBPK model that can facilitate the prediction of the systemic and local brain pharmacokinetics of a given molecule from in vitro nasal cast deposition data in humans (which can then be retrained to model non-human primates (NHPs)). In this initial study, a model was designed, built and parameterised with data from literature or estimated parameters.

The objectives of the study were to:
1. Develop a PBPK model that takes in vitro nasal cast deposition data as input and outputs plasma concentration vs. time, CSF concentration vs. time and brain tissue concentration vs. time profiles.
2. Parameterize the model using in vitro, in vivo and clinical data from literature and estimated parameters.
3. Demonstrate the predictions that the model can produce using estimated input parameters.

### PBPK Model

A multi-compartment PBPK model as described herein was used assuming well-stirred tissue and blood compartments (i.e. uniform distribution with each compartment) representing arterial blood, lung, adipose, bone, brain, heart, kidney, nose, muscle, skin, gut, spleen, liver, rest of body compartments, and venous blood. Clearance was assumed to occur in the liver and blood compartments. The nasal cavity was represented as an epithelial stack composed of nose area, nasal floor, turbinates, olfactory region and rhinopharynx regions. Each region comprised of a mucus layer where the drug was assumed to be deposited, an epithelial layer which permitted permeation into deeper tissue and a subepithelial layer perfused by blood vessels allowing transport of drug into systemic circulation. See, e.g., Example 1 for some description and FIGS. 1 and 2. The brain was represented by brain blood outside of the blood brain barrier, brain mass representing the bulk of the brain tissue, cranial CSF and spinal CSF. There were mass transport processes from the olfactory epithelium and subepithelium to the cranial CSF representing a combination of intra- and extra-cellular transport. Transport of material from the brain blood into the CSF and brain mass was modelled following L. F. Verscheijden, J. B. Koenderink, S. N. de Wildt and F. G. Russel, “Development of a physiologically-based pharmacokinetic pediatric brain model for prediction of cerebrospinal fluid drug concentrations and the influence of meningitis,” PLOS Computational Biology, vol. 15, p. e1007117, 2019 where the region beyond the blood brain barrier (BBB) and blood CSF barrier (BCSFB) is separated into brain mass, cranial CSF and spinal CSF regions. A schematic of the nasal part of the model is shown in FIG. 3. Finally, the transport from the olfactory region of the nasal epithelium to the brain (beyond the BBB and BCSFB) was modelled as a single first order transport process dictated by transfer in and transfer out coefficients. Both intracellular (endocytosis and transport through the olfactory and trigeminal nerves) and extracellular (diffusion through tight junctions into the perineural space) were considered, however since it is difficult to separate these mechanisms, they were considered as a single process transporting mass from the olfactory region to the cranial CSF (T. P. Crowe, M. H. W. Greenlee, A. G. Kanthasamy and W. H. Hsu, “Mechanism of intranasal drug delivery directly to the brain,” Life Sciences, vol. 195, pp. 44-52, 2018). Since the olfactory neuronal cells penetrate the entire depth of the epithelium, transport from both the epithelial and subepithelial compartments of the olfactory region was considered. A schematic of the olfactory region of the nose and its transport pathway to the brain is shown in FIG. 4.

The model was parameterized using physiological values from literature and compound specific parameters for DHE (see Table 1 above for a list of physiological parameters and values and see Table 9 below for a list of compound specific parameters for DHE). Any parameters that were unknown were estimated at this time as the purpose of this study was simply to construct a working PBPK model. The schematic of the multi-compartment model is detailed in FIG. 1. The equations defining the model are outlined in the sub-section entitled “Compartments” under the section entitled “Model Building” in the Detailed Description above. The mathematical equations describing the models were solved using Python version 3.10.4 and SciPy 1.8.1.

TABLE 9
Sources and Values of Initial Model Compound-Specific Parameters

| Parameter | Value | Source |
|---|---|---|
| K_{pu, adipose} - Tissue: plasma | 6.35 | Estimated using the method of T. |
| partition coefficient for the | | Rodgers, D. Leahy and M. Rowland, |
| lung | | “Physiologically based pharmacokinetic |
| | | modeling 1: Predicting the tissue |
| | | distribution of moderate-to-strong bases, ” |
| | | Journal of Pharmaceutical Sciences, vol. |
| | | 94, pp. 1259-1276, 2005 using the LogP |
| | | and fraction unbound in plasma detailed |
| | | in this table |
| K_{pu, bone} - Tissue: plasma | 7.90 | Same as K_{pu, adipose} |
| partition coefficient for the | | |
| bone | | |
| K_{pu, brain} - Tissue: plasma | 6.98 | Same as K_{pu, adipose} |
| partition coefficient for the | | |
| brain | | |
| K_{pu, heart} - Tissue: plasma | 9.92 | Same as K_{pu, adipose} |
| partition coefficient for the | | |
| luheartng | | |
| K_{pu, kidney} - Tissue: plasma | 4.87 | Same as K_{pu, adipose} |
| partition coefficient for the | | |
| kidney | | |
| K_{pu, muscle} - Tissue: plasma | 2.33 | Same as K_{pu, adipose} |
| partition coefficient for the | | |
| muscle | | |
| K_{pu, skin} - Tissue: plasma | 5.81 | Same as K_{pu, adipose} |
| partition coefficient for the | | |
| skin | | |
| K_{pu, gut} - Tissue: plasma | 6.01 | Same as K_{pu, adipose} |
| partition coefficient for the | | |
| gut | | |
| K_{pu, spleen} - Tissue: plasma | 2.50 | Same as K_{pu, adipose} |
| partition coefficient for the | | |
| spleen | | |
| K_{pu, liver} - Tissue: plasma | 4.79 | Same as K_{pu, adipose} |
| partition coefficient for the | | |
| liver | | |
| K_{pu, lung} - Tissue: plasma | 1.25 | Same as K_{pu, adipose} |
| partition coefficient for the | | |
| lung | | |
| K_{pu, nose} - Tissue: plasma | 1.25 | Same as K_{pu, adipose} |
| partition coefficient for the | | |
| nose | | |
| K_{pu, nose} rest- | 4.87 | Same as K_{pu, adipose} |
| Tissue: plasma partition | | |
| coefficient for the rest of | | |
| the body | | |
| pKa | 9.71 | https://go.drugbank.com/drugs/DB00320 |
| | (acid), | |
| | 8.39 | |
| | (base) | |
| LogP | 2 | https://pubchem.ncbi.nlm.nih.gov/ |
| | | compound/Dihydroergotamine |
| fᵤ - Fraction unbound in | 0.03 | FDA, “Migranal Product Label,” [Online]. |
| blood plasma | | Available: |
| | | https://www.accessdata.fda.gov/drugsatfda_ |
| | | docs/label/2019/020148Orig1s0251bl.pdf. |
| | | [Accessed 13 Dec. 2022] |
| Clₕ - Hepatic Clearance | 30, 000 | Estimated |
| | min⁻¹ | |
| P - Permeability in the | 1.48 × 10⁻⁷ | N. Fransén, U. Espefält Westin, C. |
| nasal epithelium | | Nyström and E. Björk, “The in vitro |
| | | transport of dihydroergotamine across |
| | | porcine nasal respiratory and olfactory |
| | | mucosa and the effect of a novel powder |
| | | formulation,” Journal of Drug Delivery |
| | | Science and Technology, vol. 14, no. 4, |
| | | pp. 267-271, 2007 |

Note that a number of parameters were estimated at this stage, therefore the numerical values predicted by the model have no credibility whatsoever and were used only to explore the possible model outputs. The model parameters that were estimated are summarised in Table 10.

TABLE 10
Parameters that were Estimated for the Model Demonstration

| Parameter | Parameter Type | Value | Source |
|---|---|---|---|
| R - Blood: plasma | Compound | 1 | No source |
| ratio | | | |
| f_{u, bm} - Fraction | Compound | 0.03 | Assumed equal to |
| unbound in brain | | | fraction unbound in |
| mass | | | blood plasma (see |
| | | | Section A in |
| | | | Detailed Description |
| | | | above) |
| f_{u, bm} - Fraction | Compound | 0.03 | Assumed equal to |
| unbound in brain | | | fraction unbound in |
| mass | | | blood plasma (see |
| | | | Section A in |
| | | | Detailed Description |
| | | | above) |
| f_{u, ccsf} - Fraction | Compound | 0.03 | Assumed equal to |
| unbound in cranial | | | fraction unbound in |
| CSF | | | blood plasma (see |
| | | | Section A in |
| | | | Detailed Description |
| | | | above) |
| f_{u, scsf} - Fraction | Compound | 0.03 | Assumed equal to |
| unbound in cranial | | | fraction unbound in |
| | | | blood plasma (see |
| | | | Section A in |
| | | | Detailed Description |
| | | | above) |
| P_{b}A_{b} - | Compound | 1.875 L/hr | Value for |
| Permeability- | | | paracetamol in |
| surface area | | | humans L. F. |
| product between | | | Verscheijden, J. B. |
| brain blood and | | | Koenderink, S. N. |
| brain mass | | | de Wildt and F. G. |
| | | | Russel, |
| | | | “Development of a |
| | | | physiologically- |
| | | | based |
| | | | pharmacokinetic |
| | | | pediatric brain |
| | | | model for prediction |
| | | | of cerebrospinal |
| | | | fluid drug |
| | | | concentrations and |
| | | | the influence of |
| | | | meningitis,” PLOS |
| | | | Computational |
| | | | Biology, vol. 15, p. |
| | | | e1007117, 2019 |
| P_{c}A_{c} - | Compound | 0.9375 L/hr | Half of P_{b}A_{b} as |
| Permeability- | | | discussed in L. F. |
| surface area | | | Verscheijden, J. B. |
| product between | | | Koenderink, S. N. |
| brain blood and | | | de Wildt and F. G. |
| cranial CSF | | | Russel, |
| | | | “Development of a |
| | | | physiologically- |
| | | | based |
| | | | pharmacokinetic |
| | | | pediatric brain |
| | | | model for prediction |
| | | | of cerebrospinal |
| | | | fluid drug |
| | | | concentrations and |
| | | | the influence of |
| | | | meningitis,” PLOS |
| | | | Computational |
| | | | Biology, vol. 15, p. |
| | | | e1007117, 2019 |
| PₑAₑ - | Compound | 300 L/hr | Set as a high |
| Permeability- | | | number to simulate |
| surface area | | | no barrier to |
| product between | | | permeation (as |
| brain mass and | | | discussed in L. F. |
| cranial CSF | | | Verscheijden, J. B. |
| | | | Koenderink, S. N. |
| | | | de Wildt and F. G. |
| | | | Russel, |
| | | | “Development of a |
| | | | physiologically- |
| | | | based |
| | | | pharmacokinetic |
| | | | pediatric brain |
| | | | model for prediction |
| | | | of cerebrospinal |
| | | | fluid drug |
| | | | concentrations and |
| | | | the influence of |
| | | | meningitis,” PLOS |
| | | | Computational |
| | | | Biology, vol. 15, p. |
| | | | e1007117, 2019). |
| k_{brain, in} - Olfactory | Compound | 1 min⁻¹ | Estimated |
| to cranial CSF | | | |
| transfer coefficient | | | |
| k_{brain, in} - Cranial | Compound | 1 min⁻¹ | Estimated |
| CSF to olfactory | | | |
| transfer coefficient | | | |

To ensure that the model was implemented correctly, three simulations were conducted. One with initial masses in all regions of the body set to zero, a second with an initial intravenous bolus dose giving a 1 ng/mL plasma concentration and a third with an initial dose giving a concentration in the olfactory mucus of 1 ng/ml.

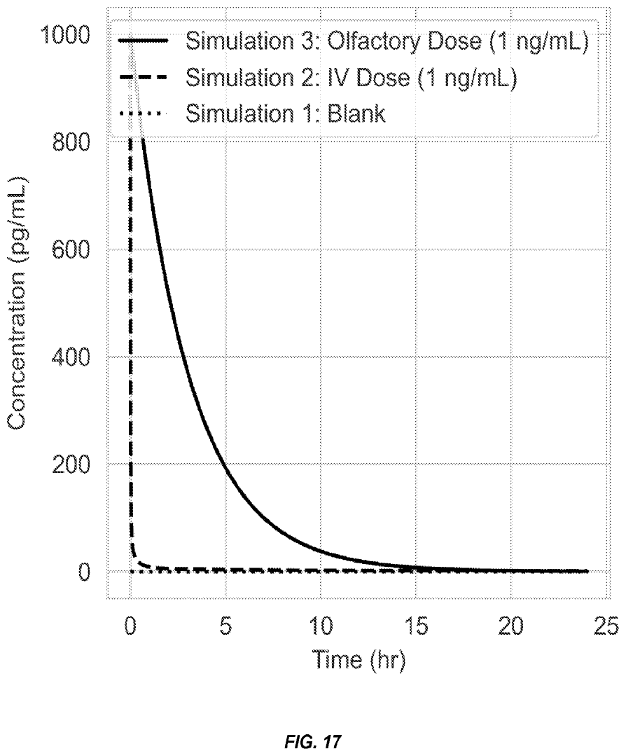

If the model was correctly implemented then the first case should have produced zeros in all compartments (including systemic plasma), the second case should have shown a multi-exponential decay from 1 ng/mL in plasma concentration and the third case should have shown multi-exponential decays to zero from the olfactory mucus compartment. The results of these three simulations in shown in FIG. 17.

These simulations demonstrated that the model correctly reproduces three expected behaviours: zero concentration is predicted if no mass is input into the model, concentration in the blood plasma decays to zero as the DHE is metabolised and cleared and concentration in the olfactory mucus decays to zero as DHE diffuses and permeates into the nasal epithelium. The model reproduces all three of these expected behaviours demonstrating its validity.

Following successful building and verification of the model behaviour (see above), a number of example simulations were conducted to demonstrate the predictions that can be made using the model. A 0.5 mg dose representing that of a single spray from Migranal (DHE nasal spray) (FDA, “Migranal Product Label,” [Online]. Available online: accessdata.fda.gov/drugsatfda_docs/label/2019/020148Orig1 s025lb1.pdf was simulated using two different deposition patterns: a high olfactory deposition pattern and a low olfactory deposition pattern. These were taken from Example 1 based on in vitro nasal cast deposition data and are summarised in Table 11.

TABLE 11
Nasal Deposition Patterns Input into the Model

| | Nose | Nasal | | Olfactory | |
|---|---|---|---|---|---|
| Deposition | Area | Floor | Turbinates | Region | Rhinopharynx |
| Pattern | Fraction | Fraction | Fraction | Fraction | Fraction |
| High | 0.06 | 0.00 | 0.34 | 0.60 | 0.00 |
| Olfactory | | | | | |
| Deposition | | | | | |
| Low | 0.13 | 0.06 | 0.75 | 0.05 | 0.01 |
| Olfactory | | | | | |
| Deposition | | | | | |

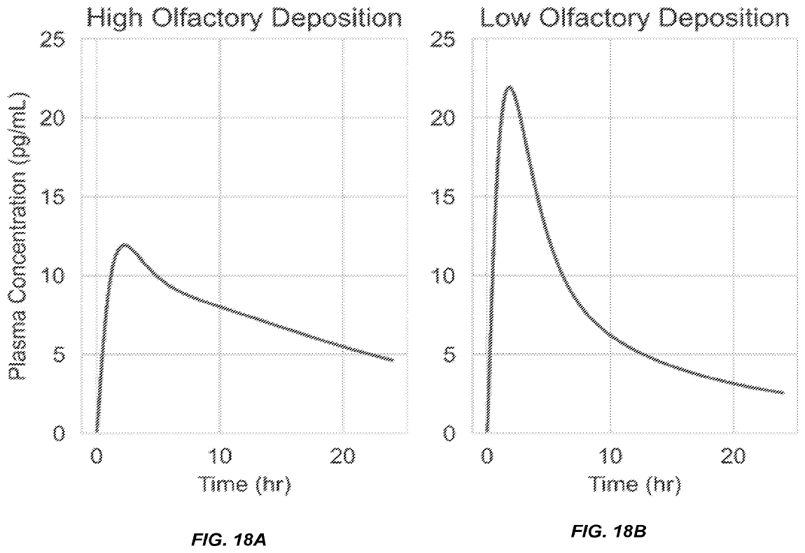

The predicted systemic plasma concentrations from the two deposition patterns are presented in FIG. 18.

The predicted systemic maximum concentrations (Cₘₐₓ) for the high olfactory deposition was 11.91 μg/mL which was lower than the 21.95 μg/mL for the low olfactory deposition. The area under the concentration vs. time curves (AUC) were similar between the two at 180.24 μg hr/mL and 180.29 μg hr/mL for the high and low olfactory deposition patterns, respectively. The predicted time to Cₘₐₓ (Tₘₐₓ) was greater for the high olfactory deposition simulations at 3.53 hr compared to 1.83 hr for the high and low olfactory deposition patterns, respectively. These trends follow the expected trend that higher deposition in the turbinates produces greater systemic exposure of drug (see Example 1 above). In addition, the same total systemic dose was available (similar AUCs) but was released over different time scales for the different levels of olfactory deposition. It should be noted that these findings are for this specific case and may vary when uncertain parameters are refined and could change for different molecules.

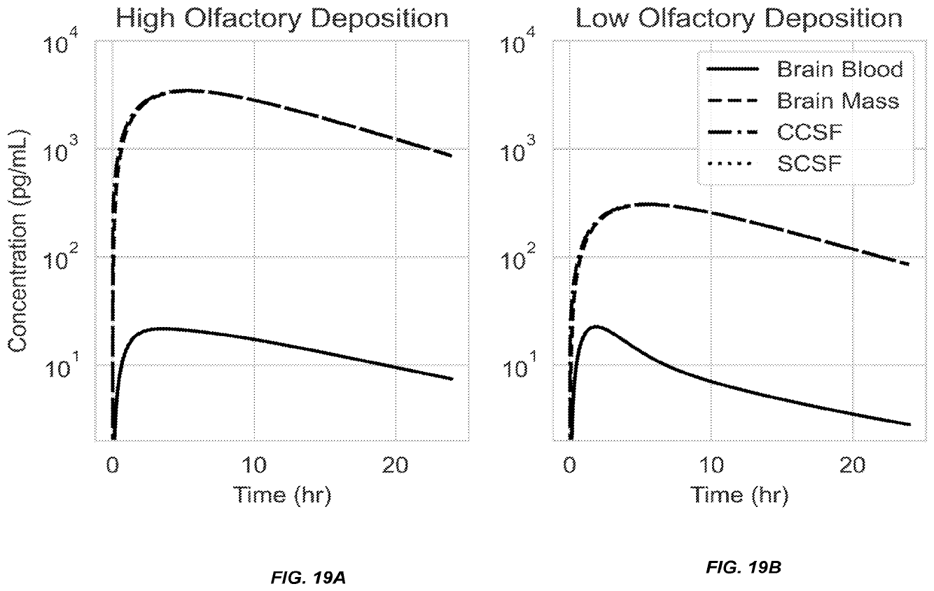

In order to determine the effect of olfactory deposition on the predicted brain exposure, the predicted concentrations of DHE in the brain blood, brain mass, cranial CSF and spinal CSF were also plotted and are presented in FIG. 19.

For both the high and the low olfactory deposition cases, the olfactory-to-cranial CSF mass transport pathway (captured by the k_{brain,in} and k_{brain,out} parameters representing a combination of both intra- and extra-cellular transport mechanisms) results in significant concentrations of DHE across all regions of the brain. The predicted brain region Cₘₐₓ values are presented in Table 12.

TABLE 12
Predicted Brain Cmax Values

| | | Brain Mass | | Spinal |
|---|---|---|---|---|
| Deposition | Brain Blood | Cₘₐₓ (pg/ | Cranial CSF | CSF Cₘₐₓ |
| Pattern | Cₘₐₓ (pg /mL) | mL) | Cₘₐₓ (pg /mL) | (pg /mL) |
| High | 21.64 | 3415.45 | 3439.56 | 3439.33 |
| Olfactory | | | | |
| Deposition | | | | |
| Low | 22.56 | 304.58 | 306.67 | 306.65 |
| Olfactory | | | | |
| Deposition | | | | |

Whilst there is only a small difference in the predicted Cₘₐₓ in the brain blood, Cₘₐₓ in the brain mass and both CSF compartments are more than an order of magnitude greater in the high olfactory deposition case than the low olfactory deposition case. This suggests that, within the highly uncertain parameters of this initial model demonstration, an increase in olfactory deposition has the potential to significantly improve targeting to the brain due to direct penetration of the blood brain barrier through the olfactory region of the nose. This contrasts with the low olfactory deposition case which relies on systemic exposure through the turbinates, transfer into the brain blood and permeation through the blood brain barrier to achieve brain exposure. The latter route, within the highly uncertain parameters of the model in its current form, appears to be much less effective as demonstrated in FIG. 19 and Table 12.

A PBPK model including brain blood, brain tissue mass and two CSF compartments in addition to a mass transfer pathway from the nasal olfactory epithelium and subepithlium to the brain was successfully designed, and implemented. A range of simple theoretical cases were tested to ensure the model produced the expected behaviour.

### Example 3: Non-Human PBPK Model

Validation of PBPK model predictions of CSF and brain mass concentrations in humans is either impossible or is considered unethical. However, the model can be validated against a model in an animal (such as rats, dogs, cats, rabbits, non-human primates, pigs, sheep, goats, cows, or horses) where CSF is sampled or a terminal study is conducted and brain mass concentration measured.

To do this, the model would be parameterized for the animal. Specifically, physiological differences would be adjusted for tissue masses and blood flows as these are known for many species (such as those mentioned above, i.e., rats, dogs, non-human primates. Additionally, the model would be parameterized for drug-specific differences from obtaining different values for Clₕ and potentially other parameters.

The parameters would be refined to ensure the model reproduces the observed blood plasma concentrations, cerebrospinal fluid concentrations and/or brain mass concentrations. This would include determination of the unknown brain transport parameters PSₑ, PS_{c} and PS_{b} which are the products of different surface areas within the brain and the permeability of the drug across the various membranes and the parameters k_{brain,in} and k_{brain,out} which are the transport coefficients from the nose-to-brain and brain-to-nose respectively.

The brain transport parameters would be assumed to be the same in the animal species vs. humans (unless surface area ratios were known then the parameters could be scaled). The rest of the physiological and drug specific parameters would be changed to those for humans. The model could then predict the concentration of drug in the CSF and brain tissue over time following a nasal dose. The model could also predict what proportion of the dose reaching the brain was transported directly from the nose through the olfactory and trigeminal nerves and what proportion first was absorbed into the blood stream and then passes the blood brain barrier.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A computer-implemented method of generating a trained nose-to-brain physiologically based pharmacokinetic (PBPK) model of delivery of a drug to brain tissue via nasal inhalation, the method comprising:
(a) generating a multi-compartment PBPK model comprising a plurality of separate body compartments;
(b) adding to the multi-compartment PBPK model a brain compartment and a nasal cavity compartment comprising one or more separate nasal sub-compartments, thereby generating an initial nose-to-brain PBPK model;
(c) parameterizing the initial nose-to-brain PBPK model to include data for a plurality of physiological parameters for the plurality of separate body compartments and/or one or more or all of the separate nasal sub-compartments;
(d) parameterizing the initial nose-to-brain PBPK model to include compound-specific parameters for a drug with known pharmacokinetic data for each body compartment and each nasal sub-compartment, to generate a parameterized nose-to-brain PBPK model;
(e) running the parameterized nose-to-brain model and generating a report comprising drug concentration data for one or more of the body compartments and/or one or more of the separate nasal sub-compartments at one or more time points; and
(f) analyzing and comparing the drug concentration data in the report with the known pharmacokinetic data for the drug, and updating the parameterized nose-to-brain model to obtain a trained nose-to-brain model.

2. The method of claim 1, wherein the plurality of compartments of the multi-compartment physiologically based pharmacokinetic model represent one or more or all of arterial blood, lung, adipose, bone, brain, heart, kidney, nose, muscle, skin, gut, spleen, liver, rest of body compartments, and/or venous blood.

3. The method of claim 1 or claim 2, wherein the one or more separate nasal sub-compartments represent one or more or all of: a nose area, a nasal floor, nasal turbinates, an olfactory region, or a rhinopharynx region.

4. The method of any one of claims 1-3, wherein the brain compartment comprises separate sub-compartments representing one or more or all of brain blood outside of the blood brain barrier, brain mass representing brain tissue, cranial cerebrospinal fluid (CSF), and/or spinal CSF.

5. The method of any one of claims 1-4, wherein at least one of the nasal sub-compartments are further represented as comprising a mucus layer where the drug is assumed to be deposited, an epithelial layer which permits permeation into deeper tissue, and a subepithelial layer perfused by blood vessels that allow transport of the drug into systemic circulation.

6. The method of any one of claims 1-5, wherein the physiological parameters for at least one compartment and at least one sub-compartment are selected from the parameters recited in Table 1.

7. The method of any one of claims 1-5, wherein the compound-specific parameters for at least one compartment and at least one sub-compartment are selected from the parameters recited in Table 2.

8. The method of any one of claims 1-6, wherein the compound-specific parameters for the drug comprises: P (permeability in the nasal epithelium), Log P (Log of the octanol/water partition coefficient), pKa (Negative log of the acid dissociation constant), fu (fraction of the drug dissolved in blood plasma that is unbound to plasma proteins), R (blood/plasma ratio), Clₙ (hepatic clearance), and mass deposited in at least one region of the nose.

9. The method of any one of claims 1-8, wherein the body compartments of the multi-compartment PBPK model are represented by differential equations representing one or more or all of:
(i) concentration of the drug in one or more of lung, adipose, bone, heart, kidneys, muscle, skin, or the rest of the body, and
(ii) plasma concentration of the drug in one or more or all of spleen, liver, gut, arterial plasma and/or venous with respect to time as functions of the volume of the lung tissue, and wherein
the differential equations are functions of one or more or all of the following parameters:
blood flow to the lung;
concentration of the drug in the venous plasma;
blood:plasma ratio;
tissue:plasma partition coefficient of the drug in each tissue type;
fraction of the drug unbound in tissue;
clearance rate of the drug from tissue;
volume of each tissue or plasma type;
concentrations of the drug in each tissue or plasma type;
blood flow rates to each tissue type;
hepatic clearance rate; and/or
clearance rate of the drug from the blood.

10. The method of claim 9, wherein the differential equations are one or more or all of the following:
$${V}_{lung} \frac{{dC}_{lung}}{dt} = {Q}_{lung} ( {C}_{ven} - \frac{R {C}_{lung}}{{K}_{pu , lung}} ) - \frac{{f}_{u} {Cl}_{tissue} {RC}_{lung}}{{K}_{pu , lung}}$$
$${V}_{T} \frac{{dC}_{T}}{dt} = {Q}_{T} ( {C}_{art} - \frac{R {C}_{T}}{{K}_{pu , T}} ) - \frac{{f}_{u} {Cl}_{tissue} {RC}_{T}}{{K}_{pu , T}}$$
$${V}_{spleen} \frac{{dC}_{spleen}}{dt} = {Q}_{spleen} ( {C}_{art} - \frac{R {C}_{spleen}}{{K}_{pu , spleen}} )$$
$${V}_{gut} \frac{{dC}_{gut}}{dt} = {Q}_{gut} ( {C}_{art} - \frac{R {C}_{gut}}{{K}_{pu , gut}} )$$
$${V}_{liver} \frac{{dC}_{liver}}{dt} = {Q}_{liver} {C}_{art} + {Q}_{spleen} \frac{R {C}_{spleen}}{{K}_{pu , spleen}} + {Q}_{gut} \frac{R {C}_{gut}}{{K}_{pu , gut}} - ( {Q}_{liver} + {Q}_{spleen} + {Q}_{gut} ) \frac{R {C}_{liver}}{{K}_{pu , liver}} - \frac{{f}_{u} {Cl}_{h} {RC}_{liver}}{{K}_{pu , liver}}$$
$${V}_{art} \frac{d {C}_{art}}{dt} = {Q}_{lung} ( \frac{{RC}_{lung}}{{K}_{pu , lung}} - {C}_{art} ) - {Cl}_{blood} {C}_{art} , and$$
$${V}_{ven} \frac{{dC}_{ven}}{di} = \sum {Q}_{T} ( \frac{R {C}_{T}}{{K}_{pu , T}} - {C}_{ven} ) + ( {Q}_{liver} + {Q}_{spleen} + {Q}_{gut} ) ( \frac{R {C}_{liver}}{{K}_{pu , liver}} - {C}_{ven} ) - {Cl}_{blood} {C}_{ven}$$
wherein V_{lung} is the volume of the lung tissue; C_{lung} is the concentration of the drug in the lung tissue; Q_{lung} is the blood flow to the lung; Cᵥₑₙ is the concentration of the drug in the venous plasma; R is the blood:plasma ratio; K_{pu,lung} is the tissue:plasma partition coefficient of the drug in the lung; fᵤ is the fraction of the drug unbound in tissue; Clₜᵢₛₛᵤₑ is the clearance rate of the drug from tissue; T is adipose, bone, heart, kidneys, muscle, skin, and the rest of the body; Vₛₚₗₑₑₙ is the volume of the spleen tissue; Cₛₚₗₑₑₙ is the concentration of the drug in the spleen tissue; Qₛₚₗₑₑₙ is the blood flow to the spleen; K_{pu,spleen} is the tissue:plasma partition coefficient of the drug in the spleen; V_{gut} is the volume of the gut tissue; C_{gut} is the concentration of the drug in the gut tissue; Q_{gut} is the blood flow to the gut; K_{pu,gut} is the tissue:plasma partition coefficient of the drug in the gut; Vₗᵢᵥₑᵣ is the volume of the liver tissue; Cₗᵢᵥₑᵣ is the concentration of the drug in the liver tissue; Qₗᵢᵥₑᵣ is the blood flow to the liver; K_{pu,liver} is the tissue:plasma partition coefficient of the drug in the liver; Cₐᵣₜ is the concentration of the drug in the arterial plasma; Clₙ is the hepatic clearance rate; and Cl_{blood} is the clearance from the blood.

11. The method of any one of claims 1-10, wherein the nasal cavity compartments are represented by differential equations representing one or more or all of:
(i) concentration of the drug in the nasal mucus,
(ii) concentration of the drug in the nasal epithelium, and
(iii) concentration of the drug in the nasal subepithelium, and wherein the differential equations are functions of one or more of the following parameters:
permeability in the nasal epithelium;
area of each region in the nasal cavity;
tissue:plasma partition coefficient of the drug in the nose;
mucociliary clearance rate in each region of the nasal cavity;
rate of transport from the olfactory tissue to the cerebrospinal fluid via the intracellular and extracellular pathways through the olfactory and/or trigeminal nerves;
rate of transport from the cerebrospinal fluid to the olfactory tissue via the intracellular and extracellular pathways through the olfactory and/or trigeminal nerves; and/or
fraction of the drug unbound in the cranial CSF and/or the concentration in the cranial CSF.

12. The method of claim 11, wherein the differential equations are one or more of the following:
$${V}_{muc , i} \frac{{dC}_{muc , i}}{dt} = - {PA}_{i} ( {C}_{muc , i} {f}_{u , muc} - {K}_{pu , nose} {C}_{ep , i} ) + {k}_{mcc , in , i} {C}_{muc , i - 1} - {k}_{mcc , out , i} {C}_{muc , i}$$
$${V}_{ep , i} \frac{{dC}_{ep , i}}{dt} = {PA}_{i} ( {C}_{muc , i} {f}_{u , muc} - {K}_{pu , nose} {C}_{ep , i} ) - {PA}_{i} ( {K}_{pu , nose} {C}_{ep , i} - {K}_{pu , nose} {C}_{sub , i} ) - \frac{{f}_{u} {Cl}_{tissue} {RC}_{ep , i}}{{K}_{pu , nose}} - {k}_{brain , in} {K}_{pu , nose} {C}_{ep , i} + {k}_{brain , out} {f}_{u , ccsf} {C}_{ccsf}$$
$${V}_{sub , i} \frac{{dC}_{sub , i}}{dt} = {PA}_{i} ( {K}_{pu , nose} {C}_{ep , i} - {K}_{pu , nose} {C}_{sub , i} ) - {Q}_{noise , i} ( {C}_{art} - \frac{{RC}_{sub , i}}{{K}_{pu , nose}} ) - \frac{{f}_{u} {Cl}_{tissue} {RC}_{sub , i}}{{K}_{pu , nose}} - {k}_{brain , in} {K}_{pu , nose} {C}_{sub , i} + {k}_{brain , out} {f}_{u , ccsf} {C}_{ccsf}$$
wherein V_{muc,i}, C_{muc,i}, V_{ep,i}, C_{ep,i}, V_{sub,i} and C_{sub,i} are the volume and drug concentration pairs in the nasal mucus, epithelium and sub-epithelium of the ith nasal region (referring to the nasal valve, floor, turbinates, turbinates, olfactory region and rhinopharynx); P is the permeability in the nasal epithelium; Aᵢ is the area of the ith nasal region; K_{pu,nose} is the tissue:plasma partition coefficient of the drug in the nose; k_{mcc,in,i} is the mucociliary clearance rate transporting mass into the ith region (equal to 0 for all regions apart from the rhinopharynx where it equals k_{mcc,ant}); k_{mcc,out,i} is the mucociliary clearance rate transporting mass out of the ith region (equal to k_{mcc,ant} for the nasal floor, turbinates and olfactory region and equal to k_{mcc,post} for the rhinopharynx); k_{brain,in} is the rate of transport from the olfactory tissue to the cerebrospinal fluid via the intracellular and extracellular pathways; k_{brain,out} is the rate of transport from the cerebrospinal fluid to the olfactory tissue via the intracellular and extracellular pathways (these two parameters are zero for the non-olfactory regions); f_{u,ccsf} is the fraction of the drug unbound in the cranial CSF; and C_{ccsf} is the concentration in the cranial CSF.

13. The method of any one of claims 1-12, wherein the brain compartments are represented by differential equations representing one or more or all of:
(i) concentration of the drug in the brain blood,
(ii) concentration of the drug in the brain mass,
(iii) concentration of the drug in the cranial cerebrospinal fluid, and
(iv) concentration of the drug in the spinal cerebrospinal fluid; and wherein
the differential equations are functions of one or more of the following parameters:
concentration of the drug molecule in the brain blood;
blood flow to the brain;
concentration of the compound in the arterial plasma;
fraction of the compound unbound in tissue;
clearance rate of the compound from tissue;
product of the permeability and surface area between brain blood and brain mass;
fraction of the drug unbound in the brain mass;
concentration of blood in the brain mass;
fraction of the drug unbound in the brain blood;
product of the permeability and surface area between brain blood and cranial CSF;
fraction of the drug unbound in the cranial CSF;
concentration of the drug in the cranial CSF the flow rates from the cranial and spinal CSF to the brain blood;
concentration of the drug in the spinal CSF;
volume of the brain mass;
product of the permeability and surface area between brain mass and cranial CSF;
bulk flow from the brain mass to the cranial CSF;
CSF shuttle flow between the cranial and spinal CSF;
rate of transport from the olfactory tissue to the cerebrospinal fluid via the intracellular and extracellular pathways along the olfactory and/or trigeminal nerves;
tissue:plasma partition coefficient of the drug in the nose;
the drug concentration in the epithelium of each region of the nasal cavity;
rate of transport from the cerebrospinal fluid to the olfactory tissue via the intracellular and extracellular pathways along the olfactory and/or trigeminal nerves; and/or
the drug concentration in the sub-epithelium of each region of the nasal cavity and/or the volume of the spinal CSF.

14. The method of claim 13, wherein the differential equations are:
$${V}_{brain} \frac{{dC}_{brain}}{dt} = {Q}_{brain} ( {C}_{art} - {RC}_{brain} ) - {f}_{u} {Cl}_{tissue} {RC}_{brain} + {P}_{b} {A}_{b} ( {f}_{u , bm} {C}_{bm} - {f}_{u , brain} {C}_{brain} ) {P}_{c} {A}_{c} ( {f}_{u , ccsf} {C}_{ccsf} - {f}_{u , brain} {C}_{brain} ) + {Q}_{ssink} {C}_{scsf} + {Q}_{csink} {C}_{ccsf}$$
$${V}_{bm} \frac{{dC}_{bm}}{dt} = {P}_{b} {A}_{b} ( {f}_{u , brain} {C}_{brain} - {f}_{u , bm} {C}_{bm} ) + {P}_{e} {A}_{e} ( {f}_{u , ccsf} {C}_{ccsf} - {f}_{u , bm} {C}_{bm} ) - {Q}_{bulk} {C}_{bm}$$
$${V}_{ccsf} \frac{{dC}_{ccsf}}{dt} = {P}_{e} {A}_{e} ( {f}_{u , bm} {C}_{bm} - {f}_{u , ccsf} {C}_{ccsf} ) + {P}_{c} {A}_{c} ( {f}_{u , brain} {C}_{brain} - {f}_{u , ccsf} {C}_{ccsf} ) + {Q}_{bulk} {C}_{bm} + {Q}_{sout} {C}_{scsf} - {Q}_{sin} {C}_{ccsf} - {Q}_{csink} {C}_{ccsf} + {k}_{brain , in} {K}_{pu , nose} {C}_{ep , i} - {k}_{brain , out} {f}_{u , ccsf} {C}_{ccsf} + {k}_{brain , in} {K}_{pu , nose} {C}_{sub , i} - {k}_{brain , out} {f}_{u , ccsf} {C}_{ccsf}$$
$${V}_{scsf} \frac{{dC}_{scsf}}{dt} = {Q}_{sin} {C}_{ccsf} - {Q}_{sout} {C}_{scsf} - {Q}_{ssink} {C}_{scsf}$$
wherein V_{brain} is the volume of the brain blood; C_{brain} is the concentration of the drug molecule in the brain blood; Q_{brain} is the blood flow to the brain; Cₐᵣₜ is the concentration of the compound in the arterial plasma; fᵤ is the fraction of the compound unbound in tissue; Clₜᵢₛₛᵤₑ is the clearance rate of the compound from tissue; P_{b}A_{b} is the product of the permeability and surface area between brain blood and brain mass; f_{u,bm} is the fraction of the drug unbound in the brain mass; C_{bm} is the concentration of blood in the brain mass; f_{u,brain} is the fraction of the drug unbound in the brain blood; P_{c}A_{c} is the product of the permeability and surface area between brain blood and cranial; f_{u,cesf} is the fraction of the drug unbound in the cranial CSF; C_{ccsf} is the concentration of the drug in the cranial CSF; Q_{csink} and Qₛₛᵢₙₖ are the flows from the cranial and spinal CSF to the brain blood respectively; C_{scsf} is the concentration of the drug in the spinal CSF; V_{bm} is the volume of the brain mass; PₑAₑ is the product of the permeability and surface area between brain mass and cranial CSF; Q_{bulk} is the bulk flow from the brain mass to the cranial CSF; Qₛₒᵤₜ and Qₛᵢₙ are the CSF shuttle flow between the cranial and spinal CSF; k_{brain,in} is the rate of transport from the olfactory tissue to the cerebrospinal fluid via the intracellular and extracellular pathways; K_{pu,nose} is the tissue:plasma partition coefficient of the drug in the nose; C_{ep,i} is the drug concentration in the epithelium of the ith nasal region (referring to the nasal valve, floor, turbinates, turbinates, olfactory region and rhinopharynx); k_{brain,out} is the rate of transport from the cerebrospinal fluid to the olfactory tissue via the intracellular and extracellular pathways; C_{sub,i} is the drug concentration in the sub-epithelium of the ith nasal region (referring to the nasal valve, floor, turbinates, turbinates, olfactory region and rhinopharynx); and V_{scsf} is the volume of the spinal CSF.

15. The method of any one of claims 1-14, wherein the compound-specific parameters of step (c) are based on pre-clinical studies for the known drug.

16. The method of claim 15, wherein the pre-clinical studies comprise in vitro cell assays, in vitro protein assays (e.g., binding data), and/or in vivo studies.

17. The method of any one of claims 1-16, wherein the physiological-specific parameters are obtained from literature and/or database searches.

18. The method of any one of claims 1-17, wherein step (e) comprises solving the differential equations representing the compartments of the PBPK model.

19. The method of claim 18, wherein the solving is done with a differential equation solver.

20. The method of any one of claims 1-19, wherein the pharmacokinetic data of step (f) is based on clinical data for the known drug.

21. The method of claim 20, further comprising adjusting one or more of the physiological parameters or the compound-specific parameters thereby training the model.

22. The method of any one of claims 1-21, wherein the PBPK model is species specific.

23. The method of claim 22, wherein the species is human.

24. The method of claim 22, wherein the species is a non-human primate.

25. The method of any one of claims 1-24, wherein the known drug is sumatriptan.

26. A computer-implemented method of predicting results for transport of a new drug to brain tissue via nasal inhalation, the method comprising:
(a) accessing a trained nose-to-brain PBPK model generated using the method of any one of claims 1 to 25 and parameterizing the nose-to-brain PBPK model with compound-specific parameters known about the drug;
(b) receiving and inputting nasal cast deposition data for the drug into the trained nose-to brain PBPK model of step (a);
(c) using a differential equation solver to solve the equations of the trained nose-to-brain PBPK model;
(d) obtaining a report comprising drug concentration data from the trained nose-to-brain model for one or more or all of the compartments and one or more or all of the sub-compartments at one or more time points; and
(e) providing a prediction of transport results for the drug to brain tissue via nasal inhalation based on pharmacokinetic data for the brain compartments based on the report.

27. The method of claim 26, wherein the compound-specific parameters of step (a) are based on pre-clinical studies.

28. The method of claim 27, wherein the pre-clinical studies comprise in vitro cell assays, in vitro protein assays (e.g., binding data), and/or in vivo studies.

29. The method of any one of claims 26-28, wherein nasal cast deposition data comprises a mass of the drug deposited in each region of the nasal cast.

30. The method of any one of claims 26-29, wherein step (a) further comprises updating the nose-to-brain PBPK model using clinical data known about the drug.

31. The method of claim 30, wherein the clinical data is intravenous data, nasal data, or both.

32. A system comprising:
a memory to store instructions that are executable; and
one or more processing devices coupled to the memory, wherein the one or more processing devices are configured to execute the instructions to perform operations comprising:
(a) generating a multi-compartment PBPK model comprising a plurality of separate body compartments;
(b) adding to the multi-compartment PBPK model a brain compartment and a nasal cavity compartment comprising one or more separate nasal sub-compartments, thereby generating an initial nose-to-brain PBPK model;
(c) parameterizing the initial nose-to-brain PBPK model to include data for a plurality of physiological parameters for the plurality of separate body compartments and/or one or more or all of the separate nasal sub-compartments;
(d) parameterizing the initial nose-to-brain PBPK model to include compound-specific parameters for a drug with known pharmacokinetic data for each body compartment and each nasal sub-compartment, to generate a parameterized nose-to-brain PBPK model;
(e) running the parameterized nose-to-brain model and generating a report comprising drug concentration data for one or more of the body compartments and/or one or more of the separate nasal sub-compartments at one or more time points; and
(f) analyzing and comparing the drug concentration data in the report with the known pharmacokinetic data for the drug, and updating the parameterized nose-to-brain model to obtain a trained nose-to-brain model.

33. The system of claim 32, wherein the plurality of compartments of the muti-compartment physiologically based pharmacokinetic model represent one or more or all of arterial blood, lung, adipose, bone, brain, heart, kidney, nose, muscle, skin, gut, spleen, liver, rest of body compartments, and/or venous blood.

34. The system of any one of claim 32 or 33, wherein the brain compartment comprises separate sub-compartments representing one or more or all of brain blood outside of the blood brain barrier, brain mass representing brain tissue, cranial cerebrospinal fluid (CSF), and/or spinal CSF.

35. The system of any one of claims 32-34, wherein the nasal sub-compartments represent one or more or all of a nose area, a nasal floor, nasal turbinates, an olfactory region, and/or a rhinopharynx region and at least one is further represented as comprising a mucus layer where the drug is assumed to be deposited, an epithelial layer which permits permeation into deeper tissue, and a subepithelial layer perfused by blood vessels that allow transport of the drug into systemic circulation.

36. The system of any one of claims 32-35, wherein the physiological parameters for at least one compartment and at least one one sub-compartment are selected from the parameters recited in Table 1.

37. The system of any one of claims 32-35, wherein the compound-specific parameters for at least one compartment and at least one sub-compartment are selected from the parameters recited in Table 2.

38. The system of any one of claims 32-37, wherein the compound-specific parameters for the drug comprises: P (permeability in the nasal epithelium), Log P (Log of the octanol/water partition coefficient), pKa (Negative log of the acid dissociation constant), fu (fraction of the drug dissolved in blood plasma that is unbound to plasma proteins), R (blood/plasma ratio), Clₕ (hepatic clearance), and mass deposited in at least one region of the nose.

39. One or more non-transitory machine-readable storage media storing instructions that are executed to perform operations comprising:
(a) generating a multi-compartment PBPK model comprising a plurality of separate body compartments;
(b) adding to the multi-compartment PBPK model a brain compartment and a nasal cavity compartment comprising one or more separate nasal sub-compartments, thereby generating an initial nose-to-brain PBPK model;
(c) parameterizing the initial nose-to-brain PBPK model to include data for a plurality of physiological parameters for the plurality of separate body compartments and/or one or more or all of the separate nasal sub-compartments;
(d) parameterizing the initial nose-to-brain PBPK model to include compound-specific parameters for a drug with known pharmacokinetic data for each body compartment and each nasal sub-compartment, to generate a parameterized nose-to-brain PBPK model;
(e) running the parameterized nose-to-brain model and generating a report comprising drug concentration data for one or more of the body compartments and/or one or more of the separate nasal sub-compartments at one or more time points; and
(f) analyzing and comparing the drug concentration data in the report with the known pharmacokinetic data for the drug, and updating the parameterized nose-to-brain model to obtain a trained nose-to-brain model.

40. The one or more non-transitory machine-readable storage media storing instructions of claim 39, wherein the plurality of compartments of the muti-compartment physiologically based pharmacokinetic model represent one or more or all of arterial blood, lung, adipose, bone, brain, heart, kidney, nose, muscle, skin, gut, spleen, liver, rest of body compartments, and/or venous blood.

41. The one or more non-transitory machine-readable storage media storing instructions of any one of claim 39 or 40, wherein the brain compartment comprises separate sub-compartments representing one or more or all of brain blood outside of the blood brain barrier, brain mass representing brain tissue, cranial cerebrospinal fluid (CSF), and/or spinal CSF.

42. The one or more non-transitory machine-readable storage media storing instructions of any one of claims 39-41, wherein the nasal sub-compartments represent one or more or all of a nose area, a nasal floor, nasal turbinates, an olfactory region, and/or a rhinopharynx region and at least one is further represented as comprising a mucus layer where the drug is assumed to be deposited, an epithelial layer which permits permeation into deeper tissue, and a subepithelial layer perfused by blood vessels that allow transport of the drug into systemic circulation.

43. The one or more non-transitory machine-readable storage media storing instructions of any one of claims 39-42, wherein the physiological parameters for at least one compartment and at least one sub-compartment are selected from the parameters recited in Table 1.

44. The one or more non-transitory machine-readable storage media storing instructions of any one of claims 39-42, wherein the compound-specific parameters for at least one compartment and at least one sub-compartment are selected from the parameters recited in Table 2.

45. The one or more non-transitory machine-readable storage media storing instructions of any one of claims 39-42, wherein the compound-specific parameters for the drug comprises: P (permeability in the nasal epithelium), Log P (Log of the octanol/water partition coefficient), pKa (Negative log of the acid dissociation constant), fu (fraction of the drug dissolved in blood plasma that is unbound to plasma proteins), R (blood/plasma ratio), Clₕ (hepatic clearance), and mass deposited in at least one region of the nose.

46. A trained nose-to-brain physiologically based pharmacokinetic (PBPK) model of delivery of a drug to brain tissue via nasal inhalation, wherein the trained nose-to-brain PBPK model is (1) encoded on one or more non-transitory machine-readable storage media and is (2) generated by operations comprising:
(a) generating a multi-compartment PBPK model comprising a plurality of separate body compartments;
(b) adding to the multi-compartment PBPK model a brain compartment and a nasal cavity compartment comprising one or more separate nasal sub-compartments, thereby generating an initial nose-to-brain PBPK model;
(c) parameterizing the initial nose-to-brain PBPK model to include data for a plurality of physiological parameters for the plurality of separate body compartments and/or one or more or all of the separate nasal sub-compartments;
(d) parameterizing the initial nose-to-brain PBPK model to include compound-specific parameters for a drug with known pharmacokinetic data for each body compartment and each nasal sub-compartment, to generate a parameterized nose-to-brain PBPK model;
(e) running the parameterized nose-to-brain model and generating a report comprising drug concentration data for one or more of the body compartments and/or one or more of the separate nasal sub-compartments at one or more time points; and
(f) analyzing and comparing the drug concentration data in the report with the known pharmacokinetic data for the drug, and updating the parameterized nose-to-brain model to obtain a trained nose-to-brain model.

47. The trained nose-to-brain physiologically based pharmacokinetic (PBPK) model of claim 46, wherein the trained nose-to-brain PBPK model is configured to:
(a) receive one or more compound-specific parameters known about a drug;
(b) be solved by a differential equation; and
(c) produce a report comprising drug concentration data from the trained nose-to-brain model for one or more or all of the compartments and one or more or all of the sub-compartments at one or more time points; and
(e) providing a prediction of transport results for the drug to brain tissue via nasal inhalation based on pharmacokinetic data for the brain compartments based on the report.

48. The trained nose-to-brain physiologically based pharmacokinetic model of claim 47, wherein the plurality of compartments of the muti-compartment physiologically based pharmacokinetic model represent one or more or all of arterial blood, lung, adipose, bone, brain, heart, kidney, nose, muscle, skin, gut, spleen, liver, rest of body compartments, and/or venous blood.

49. The trained nose-to-brain physiologically based pharmacokinetic model of any one of claim 47 or 48, wherein the brain compartment comprises separate sub-compartments representing one or more or all of brain blood outside of the blood brain barrier, brain mass representing brain tissue, cranial cerebrospinal fluid (CSF), and/or spinal CSF.

50. The trained nose-to-brain physiologically based pharmacokinetic model of any one of claims 47-49, wherein the nasal sub-compartments represent one or more or all of a nose area, a nasal floor, nasal turbinates, an olfactory region, and/or a rhinopharynx region and at least one is further represented as comprising a mucus layer where the drug is assumed to be deposited, an epithelial layer which permits permeation into deeper tissue, and a subepithelial layer perfused by blood vessels that allow transport of the drug into systemic circulation.

51. The trained nose-to-brain physiologically based pharmacokinetic model of any one of claims 47-50, wherein the physiological parameters for at least one compartment and at least one sub-compartment are selected from the parameters recited in Table 1.

52. The trained nose-to-brain physiologically based pharmacokinetic model of any one of claims 47-50, wherein the compound-specific parameters for at least one compartment and at least one sub-compartment are selected from the parameters recited in Table 2.

53. The trained nose-to-brain physiologically based pharmacokinetic model of any one of claims 47-52, wherein the compound-specific parameters for the drug comprises: P (permeability in the nasal epithelium), Log P (Log of the octanol/water partition coefficient), pKa (Negative log of the acid dissociation constant), fu (fraction of the drug dissolved in blood plasma that is unbound to plasma proteins), R (blood/plasma ratio), Clₕ (hepatic clearance), and mass deposited in at least one region of the nose.

54. A system comprising:
a memory to store instructions that are executable; and
one or more processing devices coupled to the memory, the one or more processing devices configured to execute the instructions to perform operations comprising:
(a) accessing a trained nose-to-brain PBPK model generated using the method of any one of claims 1 to 25 and parameterizing the nose-to-brain PBPK model with compound-specific parameters known about a drug;
(b) receiving and inputting nasal cast deposition data for the drug into the trained nose-to brain PBPK model of step (a);
(c) using a differential equation solver to solve the equations of the trained nose-to-brain PBPK model;
(d) obtaining a report comprising drug concentration data from the trained nose-to-brain model for one or more or all of the compartments and one or more or all of the sub-compartments at one or more time points; and
(e) providing a prediction of transport results for the drug to brain tissue via nasal inhalation based on pharmacokinetic data for the brain compartments based on the report.

55. The system of claim 54, wherein the plurality of compartments of the muti-compartment physiologically based pharmacokinetic model represent one or more or all of arterial blood, lung, adipose, bone, brain, heart, kidney, nose, muscle, skin, gut, spleen, liver, rest of body compartments, and/or venous blood.

56. The system of any one of claim 54 or 55, wherein the brain compartment comprises separate sub-compartments representing one or more or all of brain blood outside of the blood brain barrier, brain mass representing brain tissue, cranial cerebrospinal fluid (CSF), and/or spinal CSF.

57. The system of any one of claims 54-56, wherein the nasal sub-compartments represent one or more or all of a nose area, a nasal floor, nasal turbinates, an olfactory region, and/or a rhinopharynx region and at least one is further represented as comprising a mucus layer where the drug is assumed to be deposited, an epithelial layer which permits permeation into deeper tissue, and a subepithelial layer perfused by blood vessels that allow transport of the drug into systemic circulation.

58. The system of any one of claims 54-57, wherein the physiological parameters for at least one compartment and at least one sub-compartment are selected from the parameters recited in Table 1.

59. The system of any one of claims 54-57, wherein the compound-specific parameters for at least one compartment and at least one sub-compartment are selected from the parameters recited in Table 2.

60. The system of any one of claims 54-59, wherein the compound-specific parameters for the drug comprises: P (permeability in the nasal epithelium), Log P (Log of the octanol/water partition coefficient), pKa (Negative log of the acid dissociation constant), fu (fraction of the drug dissolved in blood plasma that is unbound to plasma proteins), R (blood/plasma ratio), Clₕ (hepatic clearance), and mass deposited in at least one region of the nose.

61. One or more non-transitory machine-readable storage media storing instructions that are executed to perform operations comprising:
(a) accessing a trained nose-to-brain PBPK model generated using the method of any one of claims 1 to 25 and parameterizing the nose-to-brain PBPK model with compound-specific parameters known about the drug;
(b) receiving and inputting nasal cast deposition data for the drug into the trained nose-to brain PBPK model of step (a);
(c) using a differential equation solver to solve the equations of the trained nose-to-brain PBPK model;
(d) obtaining a report comprising drug concentration data from the trained nose-to-brain model for one or more or all of the compartments and one or more or all of the sub-compartments at one or more time points; and
(e) providing a prediction of transport results for the drug to brain tissue via nasal inhalation based on pharmacokinetic data for the brain compartments based on the report.

62. The one or more non-transitory machine-readable storage media storing instructions of claim 61, wherein the plurality of compartments of the muti-compartment physiologically based pharmacokinetic model represent one or more or all of arterial blood, lung, adipose, bone, brain, heart, kidney, nose, muscle, skin, gut, spleen, liver, rest of body compartments, and/or venous blood.

63. The one or more non-transitory machine-readable storage media storing instructions of any one of claim 61 or 62, wherein the brain compartment comprises separate sub-compartments representing one or more or all of brain blood outside of the blood brain barrier, brain mass representing brain tissue, cranial cerebrospinal fluid (CSF), and/or spinal CSF.

64. The one or more non-transitory machine-readable storage media storing instructions of any one of claims 61-63, wherein the nasal sub-compartments represent one or more or all of a nose area, a nasal floor, nasal turbinates, an olfactory region, and/or a rhinopharynx region and at least one is further represented as comprising a mucus layer where the drug is assumed to be deposited, an epithelial layer which permits permeation into deeper tissue, and a subepithelial layer perfused by blood vessels that allow transport of the drug into systemic circulation.

65. The one or more non-transitory machine-readable storage media storing instructions of any one of claims 61-64, wherein the physiological parameters for at least one compartment and at least one sub-compartment are selected from the parameters recited in Table 1.

66. The one or more non-transitory machine-readable storage media storing instructions of any one of claims 61-64, wherein the compound-specific parameters for at least one compartment and at least one sub-compartment are selected from the parameters recited in Table 2.

67. The one or more non-transitory machine-readable storage media storing instructions of any one of claims 61-66, wherein the compound-specific parameters for the drug comprises: P (permeability in the nasal epithelium), Log P (Log of the octanol/water partition coefficient), pKa (Negative log of the acid dissociation constant), fu (fraction of the drug dissolved in blood plasma that is unbound to plasma proteins), R (blood/plasma ratio), Clₕ (hepatic clearance), and mass deposited in at least one region of the nose.
